(19) United States
(12) Patent Application Publication
(10) Pub. No.: US 2016/0264570 A1
(43) Pub. Date: Sep. 15, 2016

(54) METHOD OF BLOCKING TRANSMISSION OF MALARIAL PARASITE

(71) Applicants: THE UNITED STATES OF AMERICA, AS REPRESENTED BY THE SECRETARY, DEPARTMENT OF HEALTH AND HUMAN SERV, Bethesda, MD (US); LOYOLA UNIVERSITY OF CHICAGO, Chicago, IL (US)

(72) Inventors: John C. McKew, Boyds, MD (US); Wei Zheng, Potomac, MD (US); Kim C. Williamson, Bethesda, MD (US); Wenwei Huang, Rockville, MD (US); Wei Sun, Gaithersburg, MD (US); Takeshi Tanaka, Hakodate (JP); Seameen Jean Dehdashti, Poolesville, MD (US); Noel Terrence Southall, Potomac, MD (US); Crystal Tobin Magle, Chicago, IL (US); Xiuli Huang, Potomac, MD (US); Paresma Rasiklal Patel, Rockville, MD (US); Myunghoon Kim, Olympia, WA (US)

(21) Appl. No.: 15/036,355

(22) PCT Filed: Nov. 14, 2014
(86) PCT No.: PCT/US2014/065671

§ 371 Date: May 12, 2016

Related U.S. Application Data
(60) Provisional application No. 61/904,884, filed on Nov. 15, 2013.

(51) Int. Cl. C07D 471/04; A61K 31/496; A61K 31/4745; A61K 31/5377; A61K 45/06; A61K 31/506

(52) U.S. Cl. CPC C07D 471/04; A61K 45/06; A61K 31/506; A61K 31/4745; A61K 31/5377; A61K 31/496

(57) ABSTRACT

The invention provides a method of blocking transmission of a Plasmodium parasite and a method of treating or preventing malaria comprising administering to an animal an effective amount of a first compound of formula I: wherein A, B, R¹, R², R¹⁰, and R¹¹ are described herein, either alone or in combination with a second compound selected from elesclomol, NSC 174938, NVP-AUY922, Maduramicin, Narasin, Alvespimycin, Omacetaxine, Thiram, Zinc pyrithione, Phanquinone, Bortezomib, Salinomycin sodium, Monensin sodium, Dipyrithione, Dicyclopentamethylene-thiuram disulfide, YM155, Withaferin A, Adriamycin, Romidepsin, AZD-1 152-HQPA, CAY10581, Plicamycin, CUDC-101, Auranofin, Trametinib, GSK-458, Afatinib, and Panobinostat.

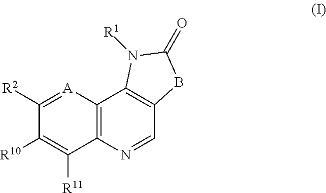

### CROSS-REFERENCE TO A RELATED APPLICATION

This patent application claims the benefit of U.S. Provisional Patent Application No. 61/904,884, filed Nov. 15, 2013, which is incorporated by reference.

### BACKGROUND OF THE INVENTION

Malaria cases and deaths have dropped 50% in 29 countries since 2000 due to the combined effects of long-lasting insecticidal bed nets, indoor residual spraying, and artemisinin-based combination therapies (ACTs) [1]. This success has raised hopes for malaria eradication and consequently stimulated interest in developing new reagents that block gametocyte transmission, such as novel and safe gametocytocidal drugs [2]. Previous drug development efforts have focused primarily on the asexual parasites that cause symptoms but not malaria transmission. To be transmitted from person to person via mosquitoes, the parasites must switch from asexual to sexual development and produce male and female gametocytes. Once gametocytes are taken up in a blood meal by a mosquito, fertilization is stimulated and the resulting zygote differentiates into a motile ookinete that migrates across the midgut epithelium of the mosquito and forms an oocyst. Over the course of the next 2 weeks, tens of thousands of infectious sporozoites are generated and sequestered in the mosquito salivary glands until released into a vertebrate host for transmission during the next blood meal.

Sexual stage P. falciparum gametocytes have a lifespan of over 3 weeks and are not cleared effectively by current antimalarial agents, except primaquine (PQ) [3,4] which is not widely used because it causes hemolytic anemia in patients with glucose-6-phosphate dehydrogenase deficiency [5]. Consequently, treatment with current antimalarial drugs often results in asymptomatic carriers who remain infectious for weeks after the clearance of asexual parasites. Despite the risks of PQ, its efficacy with artemisinin combination therapy (ACT) in reducing malaria transmission in the PQ-tolerant patients was recently demonstrated in test regions. Other than PQ, the only other gametocytocidal candidate being tested is methylene blue.

Thus, a new generation of antimalarial agents with potent activities against both sexual and asexual parasites is urgently needed for better therapeutic effect and eradication of malarial infection globally.

### BRIEF SUMMARY OF THE INVENTION

The invention provides a method of blocking transmission of a Plasmodium parasite comprising administering to a mammal in need of such treatment, a therapeutically effective amount of a first compound of formula (I):

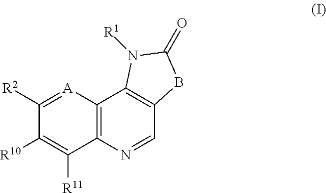

wherein A is CR¹² or N,

B is CR³═CR⁴ or NR¹³,

R¹ is an optionally substituted group selected from the group consisting of C₆₋₁₀ aryl; C₁₋₁₂ alkyl; C₁₋₁₂ alkyl wherein alkyl contains one or more oxygen, sulfur, nitrogen, phosphorus, or silicon atoms in place of carbon′ toms; 5-10-membered heteroaryl having 1-4 heteroatoms independently selected from the group consisting of nitrogen, oxygen, and sulfur; and 4-10-membered heterocyclic having 1-2 heteroatoms independently selected from the group consisting of nitrogen, oxygen, and sulfur; wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperizinyl, and 4-alkylcarbonylpiperazinyl,

R², R¹⁰, and R¹¹ are independently hydrogen, halogen, —NR⁶R⁷, —OR⁸, —SR⁹, or an optionally substituted group selected from the group consisting of C₁₋₁₂ acyl; C₆₋₁₀ aryl; C₇₋₁₅ arylalkyl; C₆₋₁₅ heteroarylalkyl; C₁₋₁₂ alkyl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; wherein the aryl or heteroaryl is optionally substituted with one or more groups selected from amino, C₁-C₆ alkylamino, di(C₁-C₆alkyl)amino, C₁-C₆ alkylcarbonylamino, C₂₋₆ alkenyl, trifluoromethyl, C₁-C₆ alkyl, halo, CN, C₁-C₆ alkoxy, alkylcarbonyl, alkylsulfonyl, hydroxyl, carboxy, C₆₋₁₀ aryl, heterocyclyl, and oxo,

R³ and R⁴ are independently selected from hydrogen, hydroxyl, OR⁵, halogen, optionally substituted C₆₋₁₀ aryl, and optionally substituted C₁₋₆ alkyl,

R⁵ is C₁₋₁₂ alkyl, and

R⁶, R⁷, R⁸, and R⁹ are independently hydrogen, an optionally substituted group selected from the group consisting of C₁₋₁₂ acyl; C₆₋₁₀ aryl; C₆₋₁₀ aryl C₁₋₁₂ alkyl; C₄₋₇ heteroaryl C₁₋₁₂ alkyl; C₁₋₁₂ alkyl; 5-10-membered heteroaryl having 1-4 heteroatoms independently selected from the group consisting of nitrogen, oxygen, and sulfur; 4-7-membered heterocyclic having 1-2 heteroatoms independently selected from the group consisting of nitrogen, oxygen, and sulfur; and C₁₋₁₂ alkyl wherein alkyl contains one or more oxygen, sulfur, nitrogen, phosphorus, or silicon atoms in place of carbon atoms; or

R⁶ and R⁷ are taken with the nitrogen atom to form a 4-7 membered heterocyclic ring having 1-2 heteroatoms independently selected from nitrogen, oxygen, and sulfur,

R¹² is hydrogen, C₁₋₁₂alkyl, C₆₋₁₀ aryl, halogen, hydroxyl, or OR⁵,

R¹³ is hydrogen, C₁₋₁₂ alkyl or C₆₋₁₀ aryl,

and/or a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat,

or a pharmaceutically acceptable salt thereof.

The invention also provides a method of treating malaria by killing or arresting the growth of Plasmodium organisms in a mammal, wherein the Plasmodium organisms are in a gametocyte stage, the method comprising administering to a mammal a therapeutically effective amount of a first compound of formula (I):

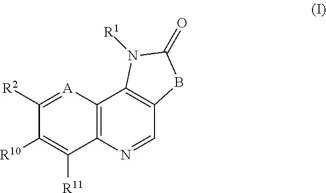

wherein A is CR¹² or N,

B is CR³═CR⁴ or NR¹³,

R¹ is an optionally substituted group selected from the group consisting of C₆₋₁₀ aryl; C₁₋₁₂ alkyl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-12-membered heterocyclic having 1-2 heteroatoms independently selected from the group consisting of nitrogen, oxygen, and sulfur; wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperizinyl, and 4-alkylcarbonylpiperazinyl,

R², R¹⁰, and R¹¹ are independently hydrogen, halogen, —NR⁶R⁷, —OR⁸, —SR⁹, or an optionally substituted group selected from the group consisting of C₁₋₁₂ acyl; C₆₋₁₀ aryl; C₇₋₁₅ arylalkyl; C₆₋₁₅ heteroarylalkyl; C₁₋₁₂ alkyl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; wherein the aryl or heteroaryl is optionally substituted with one or more groups selected from amino, C₁-C₆ alkylamino, di(C₁-C₆alkyl)amino, C₁-C₆ alkylcarbonylamino, C₂₋₆ alkenyl, trifluoromethyl, C₁-C₆ alkyl, halo, CN, C₁-C₆ alkoxy, alkylcarbonyl, alkylsulfonyl, hydroxyl, carboxy, C₆₋₁₀ aryl, heterocyclyl, and oxo,

R³ and R⁴ are independently selected from hydrogen, hydroxyl, OR⁵, halogen, optionally substituted C₆₋₁₀ aryl, and optionally substituted C₁₋₆ alkyl,

R⁵ is C₁₋₁₂ alkyl, and

R⁶, R⁷, R⁸, and R⁹ are independently hydrogen, an optionally substituted group selected from the group consisting of C₁₋₁₂ acyl; C₆₋₁₀ aryl; C₆₋₁₀ aryl C₁₋₁₂ alkyl; C₄₋₇ heteroaryl C₁₋₁₂ alkyl; C₁₋₁₂ alkyl; 5-10-membered heteroaryl having 1-4 heteroatoms independently selected from the group consisting of nitrogen, oxygen, and sulfur; 4-7-membered heterocyclic having 1-2 heteroatoms independently selected from the group consisting of nitrogen, oxygen, and sulfur; and C₁₋₁₂ alkyl wherein alkyl contains one or more oxygen, sulfur, nitrogen, phosphorus, or silicon atoms in place of carbon atoms; or

R⁶ and R⁷ are taken with the nitrogen atom to form a 4-7 membered heterocyclic ring having 1-2 heteroatoms independently selected from nitrogen, oxygen, and sulfur,

R¹² is hydrogen, C₁₋₁₂ alkyl, C₆₋₁₀ aryl, halogen, hydroxyl, or OR⁵,

R¹³ is hydrogen, C₁₋₁₂ alkyl or C₆₋₁₀ aryl,

and/or a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat,

or a pharmaceutically acceptable salt thereof.

### BRIEF DESCRIPTION OF THE SEVERAL VIEWS OF THE DRAWING(S)

FIG. 1 depicts the structures of compounds in accordance with an embodiment of the invention.

FIG. 2A illustrates a protocol for a mouse model of gametocyte transmission in accordance with an embodiment of the invention.

FIG. 2B illustrates the result of a malaria mouse model for Torin 2 using a 2 dose dosing regime.

FIG. 2C illustrates the result of a malaria mouse model for Torin 2 using a 1 dose dosing regime.

FIGS. 3A-3D illustrate dose-concentration curves of panobinostat, CUDC-101, primaquine, and Torin 2, respectively, against the drug sensitive 3D7 strain and against two asexual drug resistant strains HB3 and Dd2.

FIG. 4 illustrates the results of a gametocyte viability assay for Torin 2 and Torin 1.

FIG. 5 depicts the structures of Torin 2, Torin 1, and WWH030.

FIG. 6A illustrates a protocol for a mouse model of gametocyte transmission in accordance with an embodiment of the invention. The oocyte number for vehicle, NVP-AUY922, and alvespimycin-treated mice are depicted in FIG. 6B. The structures of NVP-AUY922 and alvespimycin are depicted in FIG. 6C.

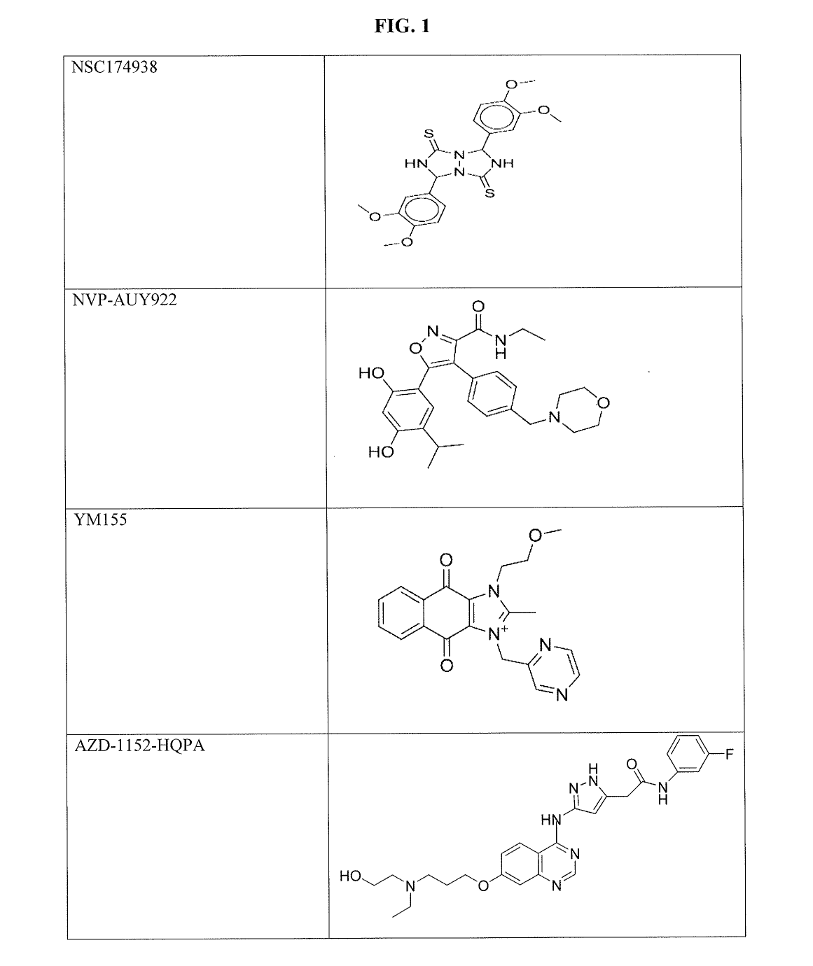

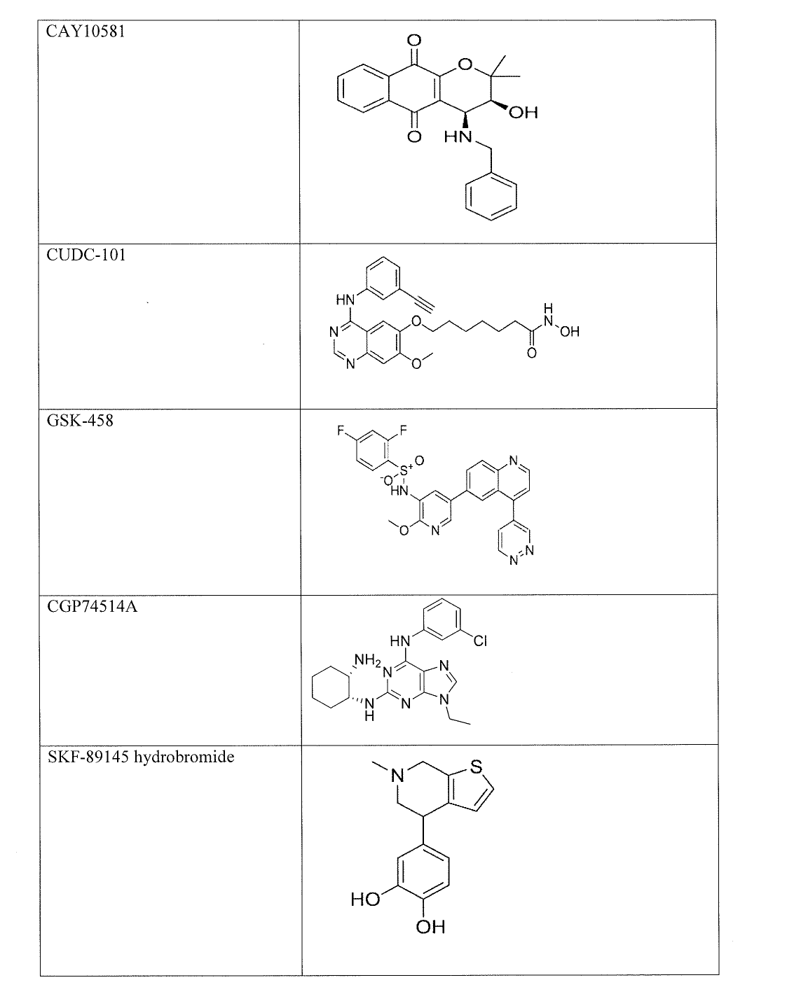

### DETAILED DESCRIPTION OF THE INVENTION

In an embodiment, the invention provides a method of blocking transmission of a Plasmodium parasite comprising administering to a mammal in need of such treatment, a therapeutically effective amount of a first compound of formula (I):

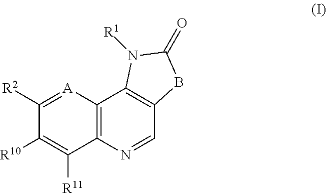

wherein A is CR¹² or N,

B is CR³═CR⁴ or NR¹³,

R¹ is an optionally substituted group selected from the group consisting of C₆₋₁₀ aryl; C₁₋₁₂ alkyl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-12-membered heterocyclic having 1-2 heteroatoms independently selected from the group consisting of nitrogen, oxygen, and sulfur; wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperizinyl, and 4-alkylcarbonylpiperazinyl,

R², R¹⁰, and R¹¹ are independently hydrogen, halogen, —NR⁶R⁷, —OR⁸, —SR⁹, or an optionally substituted group selected from the group consisting of C₁₋₁₂ acyl; C₆₋₁₀ aryl; C₇₋₁₅ arylalkyl; C₆₋₁₅ heteroarylalkyl; C₁₋₁₂ alkyl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; wherein the aryl or heteroaryl is optionally substituted with one or more groups selected from amino, C₁-C₆ alkylamino, di(C₁-C₆alkyl)amino, C₁-C₆ alkylcarbonylamino, C₂₋₆ alkenyl, trifluoromethyl, C₁-C₆ alkyl, halo, CN, C₁-C₆ alkoxy, alkylcarbonyl, alkylsulfonyl, hydroxyl, carboxy, C₆₋₁₀ aryl, heterocyclyl, and oxo,

R³ and R⁴ are independently selected from hydrogen, hydroxyl, OR⁵, halogen, optionally substituted C₆₋₁₀ aryl, and optionally substituted C₁₋₆ alkyl,

R⁵ is C₁₋₁₂ alkyl, and

R⁶, R⁷, R⁸, and R⁹ are independently hydrogen, an optionally substituted group selected from the group consisting of C₁₋₁₂ acyl; C₆₋₁₀ aryl; C₆₋₁₀ aryl C₁₋₁₂ alkyl; C₄₋₇ heteroaryl C₁₋₁₂ alkyl; C₁₋₁₂ alkyl; 5-10-membered heteroaryl having 1-4 heteroatoms independently selected from the group consisting of nitrogen, oxygen, and sulfur; 4-7-membered heterocyclic having 1-2 heteroatoms independently selected from the group consisting of nitrogen, oxygen, and sulfur; and C₁₋₁₂ alkyl wherein alkyl contains one or more oxygen, sulfur, nitrogen, phosphorus, or silicon atoms in place of carbon atoms; or

R⁶ and R⁷ are taken with the nitrogen atom to form a 4-7 membered heterocyclic ring having 1-2 heteroatoms independently selected from nitrogen, oxygen, and sulfur,

R¹² is hydrogen, C₁₋₁₂ alkyl, C₆₋₁₀ aryl, halogen, hydroxyl, or OR⁵,

R¹³ is hydrogen, C₁₋₁₂ alkyl or C₆₋₁₀ aryl,

and/or a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat,

or a pharmaceutically acceptable salt thereof.

In certain embodiments, the compound is of formula (I) and B is CR³═CR⁴.

In certain of these embodiments, A is CH or N,

R¹ is C₁-C₆ alkyl, C₆-C₁₀ aryl, or heteroaryl, wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperizinyl, and 4-alkylcarbonylpiperazinyl,

R² is C₆-C₁₀ aryl or heteroaryl, wherein the aryl or heteroaryl is optionally substituted with one or more groups selected from amino, C₁-C₆ alkylamino, di(C₁-C₆alkyl)amino, C₁-C₆ alkylcarbonylamino, sulfonyl, di(C₁-C₆ alkyl)carbonylamino, trifluoromethyl, halo, C₂-C₆ alkenyl, cyano, C₁-C₆ alkoxy, acyl, C₁-C₆ alkyl, hydroxyl, heterocyclyl, oxo, aminosulfonyl, alkylsulfonylamino, C₁-C₆ alkylaminomethyl, and di(C₁-C₆ alkyl)aminomethyl,

R¹⁰ and R¹¹ are both hydrogen, and

R³ and R⁴ are individually selected from hydrogen, halo, optionally substituted C₁-C₆ alkyl, and OR⁵.

In certain embodiments, when A is CH, B is CR³═CR⁴, R³, R⁴, R¹⁰, and R¹¹ are each hydrogen, and R¹ is 3-trifluoromethylphenyl, R² is not 2-amino-5-pyridyl or 3-quinolinyl.

Referring now to terminology used generically herein, the term “alkyl” means a straight-chain or branched alkyl substituent containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hückel's Rule.

The term “heteroaryl” refers to a monocyclic or bicyclic 5 to 10-membered ring system as described herein, wherein the heteroaryl group is unsaturated and satisfies Hacker s rule, and wherein the heteroaryl contains 1-4 heteroatoms independently selected from nitrogen, oxygen and sulfur. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiopheneyl, indolyl, indazolyl, imidazolyl, quinolinyl, isoquinolinyl, benzimidazolyl, benzoxazolinyl, benzothiazolinyl, and quinazolinyl. The heteroaryl groups can be attached at any open position on the heteroaryl groups. The terms “heterocyclic” or “heterocyclyl” refer to a 4 to 12-membered heterocyclic ring system as described herein, wherein the heterocycle contains 1 or 2 heteroatoms independently selected from nitrogen, oxygen, and sulfur, wherein the heterocycle is saturated or monounsaturated. The heterocyclyl or heteroaryl group is optionally substituted with 1, 2, 3, 4, or 5 substituents as recited herein such as with alkyl groups such as methyl groups, ethyl groups, and the like, or with aryl groups such as phenyl groups, naphthyl groups and the like, wherein the aryl groups can be further substituted with, for example halo, dihaloalkyl, trihaloalkyl, nitro, hydroxy, alkoxy, aryloxy, amino, substituted amino, alkylcarbonyl, alkoxycarbonyl, arylcarbonyl, aryloxycarbonyl, thio, alkylthio, arylthio, and the like, wherein the optional substituent can be present at any open position on the heterocyclyl or heteroaryl group.

The term “alkyl wherein alkyl contains one or more oxygen, sulfur, nitrogen, phosphorus, or silicon atoms in place of carbon atoms” refers to a linear or branched alkyl group wherein one or more carbon atoms in the alkyl group is replace with the aforesaid atoms. Non-limiting examples of alkyl wherein alkyl contains one or more oxygen, sulfur, nitrogen, phosphorus, or silicon atoms in place of carbon atoms include, for example, methoxymethyl, methoxyethyl, methylaminoethyl, and the like.

The term “acyl” refers to an alkylcarbonyl substituent. The term “alkylsulfonylamino” refers to a group of the structure: alkyl-SO₂—NH—. The term “aminosulfonyl” refers to a group of the structure: H₂NSO₂—.

In certain embodiments, the compound is a compound of formula (I) and B is CR³═CR⁴. In certain embodiments, R¹ is C₁-C₆ alkyl, C₆-C₁₀ aryl, or heteroaryl, wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperizinyl, and 4-alkylcarbonylpiperazinyl. In certain embodiments, R² is C₆-C₁₀ aryl or heteroaryl, wherein the aryl or heteroaryl is optionally substituted with one or more groups selected from amino, C₁-C₆ alkylamino, di(C₁-C₆alkyl)amino, and C₁-C₆ alkylcarbonylamino. In certain embodiments, R¹⁰ and R¹¹ are both hydrogen. In certain embodiments, R³ and R⁴ are both hydrogen. In certain preferred embodiments, A is CH. In certain preferred embodiments, R¹ is selected from 3-trifluoromethylphenyl, 4-piperazinylmethyl, ethyl, phenyl, 3-ethylphenyl, 3-chlorophenyl, 3-cyanophenyl, 3-methoxyphenyl, 3-(dimethylaminocarbonyl)phenyl, 3-sulfonamidophenyl, 3-phenoxyphenyl, 3-ethoxyphenyl, 4-(piperazin-4-yl)-3-trifluoromethylphenyl, 4-piperazinyl, 1-acetylpiperidin-4-yl,cyclopropyl, 4-tetrahydropyranyl, cyclohexyl, and cyclopentyl. In certain preferred embodiments, R² is selected from 2-amino-pyridinyl, 4-pyridinyl, 2-amino-5-pyrimidinyl, 3-pyridyl, quinolin-3-yl, 5-pyrimidinyl, 2-amino-5-trifluoromethylpyrimidin-5-yl, 2-acetylamino-5-pyridyl, 2-amino-4-methylpyrimidin-5-yl, 1-piperazinyl, indol-5-yl, 1H-indazol-5-yl, 4-aminophenyl, 1,2,3,6-tetrahydropyridin-4-yl, 1H-pyrazol-4-yl, 1H-benzo[d]imidazol-5-yl, 4-sulfonylaminophenyl, 2-dimethylaminopyrimidin-5-yl, 3-trifluoromethylphenyl, bromo, 3-aminophenyl, vinyl, 4-aminocarbonylphenyl, 3-cyanophenyl, 3-trifluoromethyl-5-pyridyl, tetrazolyl, 4-chlorophenyl, 4-methoxyphenyl, 3-aminocarbonylphenyl, 3-acetylphenyl, 2,3-dihydrobenzofuran-6-yl, 1-methyl-1H-indol-5-yl, benzo[d][1,3]dioxo-5-yl, 4-fluorophenyl, 4-hydroxyphenyl, porpholin-1-yl, benzo[b]thiophen-1-yl, 4-methylsulfonylphenyl, benzo[c][1,2,5]oxadiazol-5-yl, 2-(piperidin-1-yl)-3-pyridinyl, 4-carboxyphenyl, 2-methyl-5-pyridyl, 4-methylsulfonylphenyl, 4-dimethylaminocarbonylphenyl, 4-phenylphenyl, 4-methylpenyl, 3-chloro-5-pyridyl, (3-pyrrolidin-1-yl)phenyl, 4-([piperizin-1-yl]carbonyl)phenyl, 4-([morpholin-1-yl]carbonyl)phenyl, 2-hydroxypyrimidin-5-yl, 3-aminosulfonylphenyl, 2-oxo-1,2,3,4,tetrahydroisoquinolin-6-yl, 2-oxo-1,2,3,4,-tetrahydroquinolin-6-yl, 4-(aminomethyl)phenyl, 4-(dimethylaminomethyl)phenyl, 4-(methylaminocarbonyl)phenyl, 1-oxoindolin-5-yl, and 1-oxoisoindolin-5-yl.

In an embodiment, the compound has the formula:

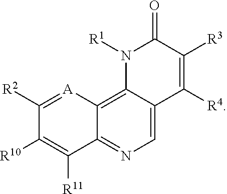

In certain preferred embodiments, the compound has the formula:

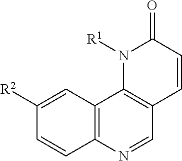

wherein R¹ and R² are:

| R¹ | R² |
|---|---|
| 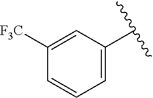 [SMILES: *c1cccc(C(F)(F)F)c1] | 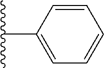 [SMILES: *c1ccccc1] |
| 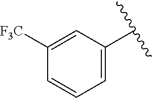 [SMILES: *c1cccc(C(F)(F)F)c1] | 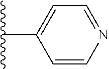 [SMILES: *c1ccncc1] |
| 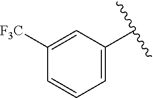 [SMILES: *c1cccc(C(F)(F)F)c1] | 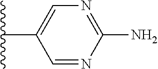 [SMILES: *c1cnc(N)nc1] |
| 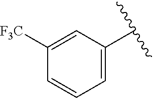 [SMILES: *c1cccc(C(F)(F)F)c1] | 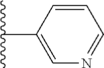 [SMILES: *c1cccnc1] |
| 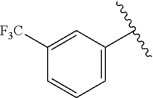 [SMILES: *c1cccc(C(F)(F)F)c1] | 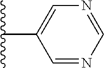 [SMILES: *c1cncnc1] |
| Ethyl | 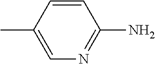 |
| 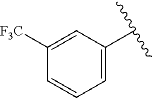 [SMILES: *c1cccc(C(F)(F)F)c1] | 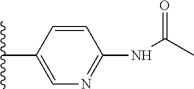 [SMILES: *c1ccc(NC(C)=O)nc1] |
| 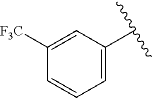 [SMILES: *c1cccc(C(F)(F)F)c1] | 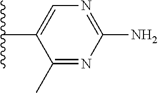 [SMILES: *c1cnc(N)nc1C] |
| 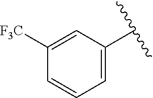 [SMILES: *c1cccc(C(F)(F)F)c1] | 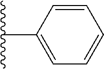 [SMILES: *c1ccccc1] |
| 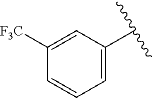 [SMILES: *c1cccc(C(F)(F)F)c1] | 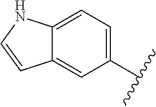 [SMILES: *c1ccc2[nH]ccc2c1] |
| 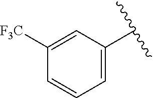 [SMILES: *c1cccc(C(F)(F)F)c1] | 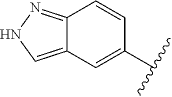 [SMILES: *c1ccc2n[nH]cc2c1] |
| 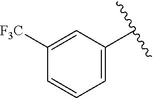 [SMILES: *c1cccc(C(F)(F)F)c1] | 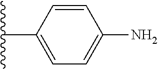 [SMILES: *c1ccc(N)cc1] |
| 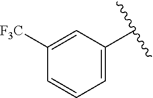 [SMILES: *c1cccc(C(F)(F)F)c1] | 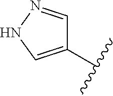 [SMILES: *c1cn[nH]c1] |
| 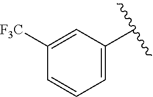 [SMILES: *c1cccc(C(F)(F)F)c1] | 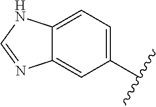 [SMILES: *c1ccc2[nH]cnc2c1] |
| 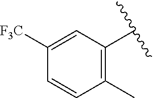 [SMILES: *c1cc(C(F)(F)F)ccc1C] | 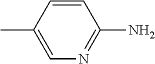 |
| 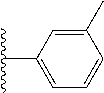 [SMILES: *c1cccc(C)c1] | 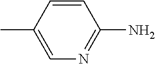 |
| 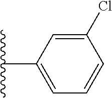 [SMILES: *c1cccc(Cl)c1] | 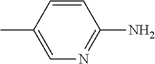 |
| 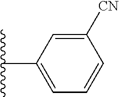 [SMILES: *c1cccc(C#N)c1] | 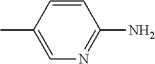 |
| 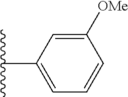 [SMILES: *c1cccc(OC)c1] | 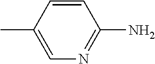 |
| 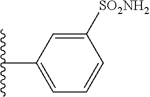 [SMILES: *c1cccc(S(N)(=O)=O)c1] | 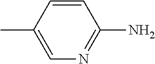 |
| 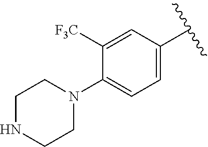 [SMILES: *c1ccc(N2CCNCC2)c(C(F)(F)F)c1] | 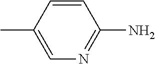 |
| 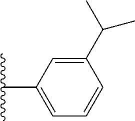 [SMILES: *c1cccc(C(C)C)c1] | 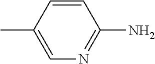 |
| 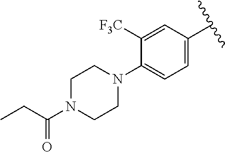 [SMILES: *c1ccc(N2CCN(C(=O)CC)CC2)c(C(F)(F)F)c1] | 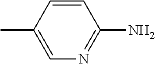 |
| 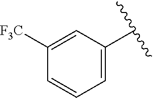 [SMILES: *c1cccc(C(F)(F)F)c1] | 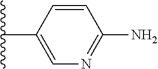 [SMILES: *c1ccc(N)nc1] |
| 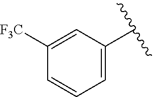 [SMILES: *c1cccc(C(F)(F)F)c1] | 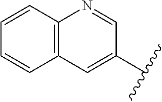 [SMILES: *c1cnc2ccccc2c1] |
| 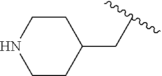 [SMILES: *CC1CCNCC1] | 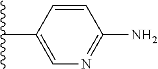 [SMILES: *c1ccc(N)nc1] |
| 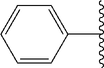 [SMILES: *c1ccccc1] | 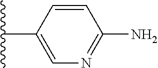 [SMILES: *c1ccc(N)nc1] |
| 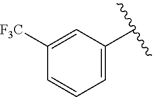 [SMILES: *c1cccc(C(F)(F)F)c1] | 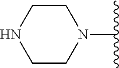 [SMILES: *N1CCNCC1] |
| 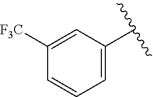 [SMILES: *c1cccc(C(F)(F)F)c1] | 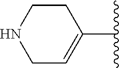 [SMILES: *C1=CCNCC1] |
| 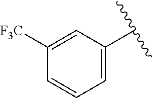 [SMILES: *c1cccc(C(F)(F)F)c1] | 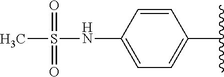 [SMILES: *c1ccc(NS(C)(=O)=O)cc1] |
| 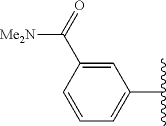 [SMILES: *c1cccc(C(=O)N(C)C)c1] | 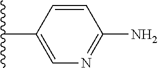 [SMILES: *c1ccc(N)nc1] |
| 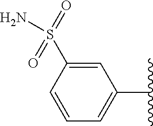 [SMILES: *c1cccc(S(N)(=O)=O)c1] | 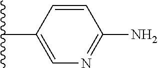 [SMILES: *c1ccc(N)nc1] |
| 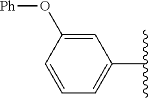 [SMILES: *c1cccc(Oc2ccccc2)c1] | 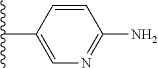 [SMILES: *c1ccc(N)nc1] |
| 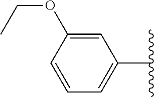 [SMILES: *c1cccc(OCC)c1] | 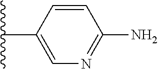 [SMILES: *c1ccc(N)nc1] |
| 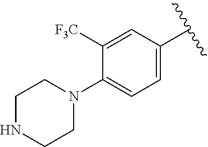 [SMILES: *c1ccc(N2CCNCC2)c(C(F)(F)F)c1] | 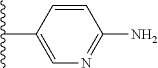 [SMILES: *c1ccc(N)nc1] |
| 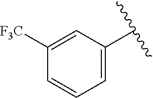 [SMILES: *c1cccc(C(F)(F)F)c1] | 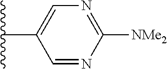 [SMILES: *c1cnc(N(C)C)nc1] |
| 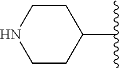 [SMILES: *C1CCNCC1] | 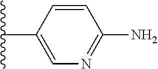 [SMILES: *c1ccc(N)nc1] |
| 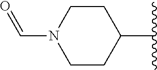 [SMILES: *C1CCN(C=O)CC1] | 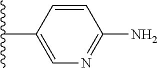 [SMILES: *c1ccc(N)nc1] |
| 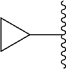 [SMILES: *C1CC1] | 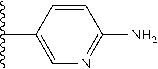 [SMILES: *c1ccc(N)nc1] |
| 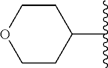 [SMILES: *C1CCOCC1] | 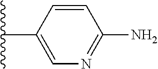 [SMILES: *c1ccc(N)nc1] |
| 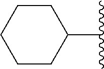 [SMILES: *C1CCCCC1] | 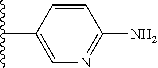 [SMILES: *c1ccc(N)nc1] |
| 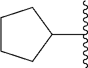 [SMILES: *C1CCCC1] | 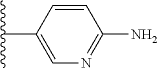 [SMILES: *c1ccc(N)nc1] |
| 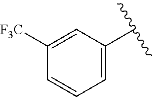 [SMILES: *c1cccc(C(F)(F)F)c1] | 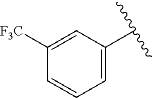 [SMILES: *c1cccc(C(F)(F)F)c1] |
| 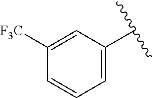 [SMILES: *c1cccc(C(F)(F)F)c1] | Br |
| 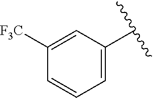 [SMILES: *c1cccc(C(F)(F)F)c1] | 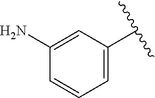 [SMILES: *c1cccc(N)c1] |
| 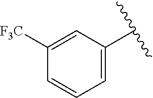 [SMILES: *c1cccc(C(F)(F)F)c1] | 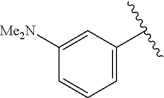 [SMILES: *c1cccc(N(C)C)c1] |
| 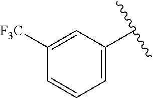 [SMILES: *c1cccc(C(F)(F)F)c1] | CH₂═CH |
| 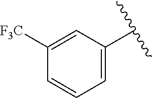 [SMILES: *c1cccc(C(F)(F)F)c1] | 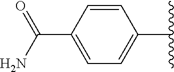 [SMILES: *c1ccc(C(N)=O)cc1] |
| 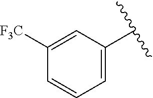 [SMILES: *c1cccc(C(F)(F)F)c1] | 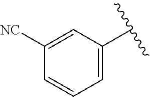 [SMILES: *c1cccc(C#N)c1] |
| 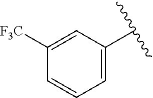 [SMILES: *c1cccc(C(F)(F)F)c1] | 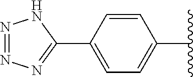 [SMILES: *c1ccc(-c2nnn[nH]2)cc1] |
| 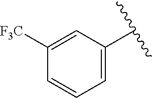 [SMILES: *c1cccc(C(F)(F)F)c1] | 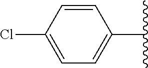 [SMILES: *c1ccc(Cl)cc1] |
| 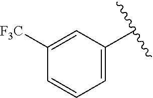 [SMILES: *c1cccc(C(F)(F)F)c1] | 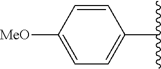 [SMILES: *c1ccc(OC)cc1] |
| 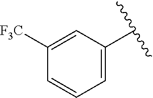 [SMILES: *c1cccc(C(F)(F)F)c1] | 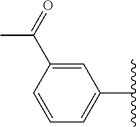 [SMILES: *c1cccc(C(C)=O)c1] |
| 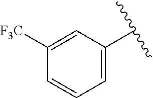 [SMILES: *c1cccc(C(F)(F)F)c1] | 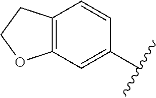 [SMILES: *c1ccc2c(c1)OCC2] |
| 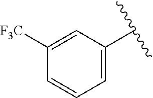 [SMILES: *c1cccc(C(F)(F)F)c1] | 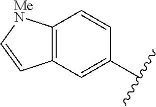 [SMILES: *c1ccc2c(ccn2C)c1] |
| 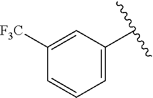 [SMILES: *c1cccc(C(F)(F)F)c1] | 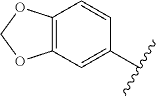 [SMILES: *c1ccc2c(c1)OCO2] |
| 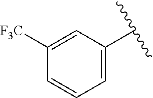 [SMILES: *c1cccc(C(F)(F)F)c1] | 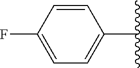 [SMILES: *c1ccc(F)cc1] |
| 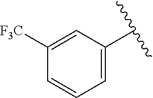 [SMILES: *c1cccc(C(F)(F)F)c1] | 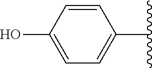 [SMILES: *c1ccc(O)cc1] |
| 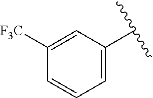 [SMILES: *c1cccc(C(F)(F)F)c1] | 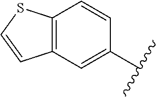 [SMILES: *c1ccc2sccc2c1] |
| 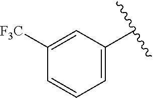 [SMILES: *c1cccc(C(F)(F)F)c1] | 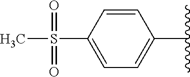 [SMILES: *c1ccc(S(C)(=O)=O)cc1] |
| 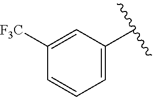 [SMILES: *c1cccc(C(F)(F)F)c1] | 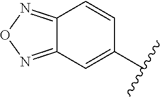 [SMILES: *c1ccc2nonc2c1] |
| 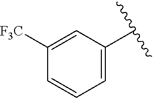 [SMILES: *c1cccc(C(F)(F)F)c1] | 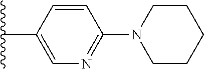 [SMILES: *c1ccc(N2CCCCC2)nc1] |
| 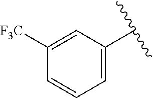 [SMILES: *c1cccc(C(F)(F)F)c1] | 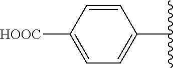 [SMILES: *c1ccc(C(=O)O)cc1] |
| 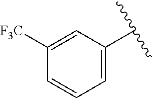 [SMILES: *c1cccc(C(F)(F)F)c1] | 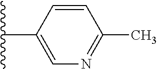 [SMILES: *c1ccc(C)nc1] |
| 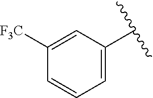 [SMILES: *c1cccc(C(F)(F)F)c1] | 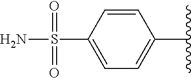 [SMILES: *c1ccc(S(N)(=O)=O)cc1] |
| 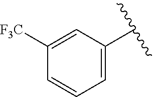 [SMILES: *c1cccc(C(F)(F)F)c1] | 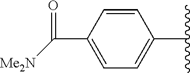 [SMILES: *c1ccc(C(=O)N(C)C)cc1] |
| 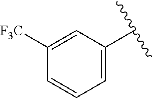 [SMILES: *c1cccc(C(F)(F)F)c1] | 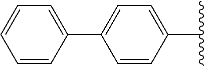 [SMILES: *c1ccc(-c2ccccc2)cc1] |
| 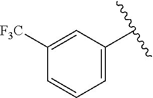 [SMILES: *c1cccc(C(F)(F)F)c1] | 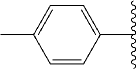 [SMILES: *c1ccc(C)cc1] |
| 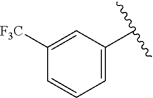 [SMILES: *c1cccc(C(F)(F)F)c1] | 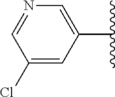 [SMILES: *c1cncc(Cl)c1] |
| 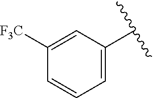 [SMILES: *c1cccc(C(F)(F)F)c1] | 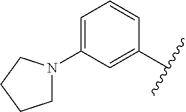 [SMILES: *c1cccc(N2CCCC2)c1] |
| 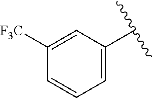 [SMILES: *c1cccc(C(F)(F)F)c1] | 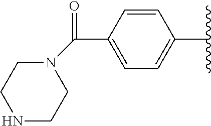 [SMILES: *c1ccc(C(=O)N2CCNCC2)cc1] |
| 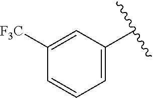 [SMILES: *c1cccc(C(F)(F)F)c1] | 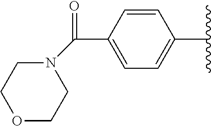 [SMILES: *c1ccc(C(=O)N2CCOCC2)cc1] |
| 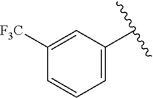 [SMILES: *c1cccc(C(F)(F)F)c1] | 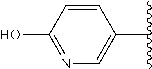 [SMILES: *c1ccc(O)nc1] |
| 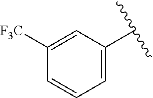 [SMILES: *c1cccc(C(F)(F)F)c1] | 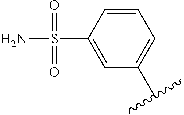 [SMILES: *c1cccc(S(N)(=O)=O)c1] |
| 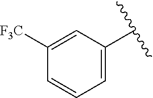 [SMILES: *c1cccc(C(F)(F)F)c1] | 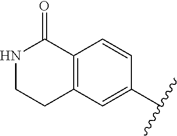 [SMILES: *c1ccc2c(c1)CCNC2=O] |
| 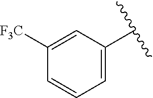 [SMILES: *c1cccc(C(F)(F)F)c1] | 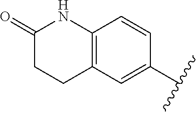 [SMILES: *c1ccc2c(c1)CCC(=O)N2] |
| 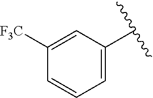 [SMILES: *c1cccc(C(F)(F)F)c1] | CH₂NH₂ |
| 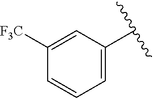 [SMILES: *c1cccc(C(F)(F)F)c1] | CH₂NMe₂ |
| 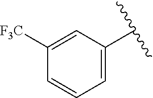 [SMILES: *c1cccc(C(F)(F)F)c1] | 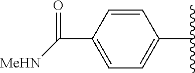 [SMILES: *c1ccc(C(=O)NC)cc1] |
| 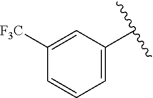 [SMILES: *c1cccc(C(F)(F)F)c1] | 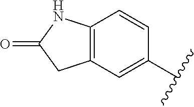 [SMILES: *c1ccc2c(c1)CC(=O)N2] |
| 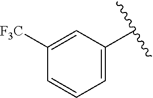 [SMILES: *c1cccc(C(F)(F)F)c1] | 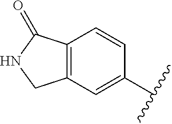 [SMILES: *c1ccc2c(c1)CNC2=O] |

In certain embodiments, the compound is

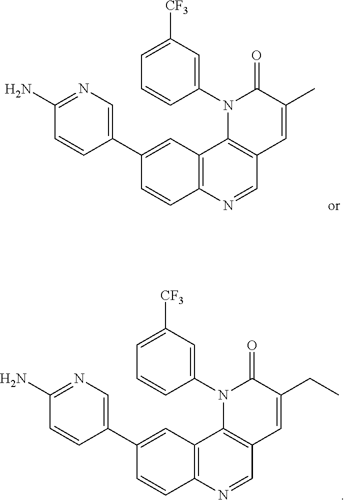 [SMILES: Cc1cc2cnc3ccc(-c4ccc(N)nc4)cc3c2n(-c2cccc(C(F)(F)F)c2)c1=O ; CCc1cc2cnc3ccc(-c4ccc(N)nc4)cc3c2n(-c2cccc(C(F)(F)F)c2)c1=O]

In certain other embodiments, the compound is a compound of formula (I) and B is NR¹². In certain preferred embodiments, A is CH. In certain embodiments, R¹⁰ and R¹¹ are both hydrogen. In certain embodiments, R¹³ is hydrogen or C₁₋₁₂ alkyl. In a certain preferred embodiment, R¹ is 3-trifluoromethylphenyl. In certain preferred embodiments, R² is selected from the group consisting of 2-methyl-5-pyridyl, 4-aminophenyl, 2-acetylamino-5-pyridyl, 4-hydroxyphenyl, 3-aminophenyl, 4-pyridyl, 1H-benzo[d]imidazol-5-yl, 4-methlsulfonylphenyl, quinolin-3-yl, 2-aminopyrimidin-5-yl, 3-cyanophenyl, 3-pyridyl, and 4-aminocarbonylphenyl.

In certain preferred embodiments, the compound is selected from

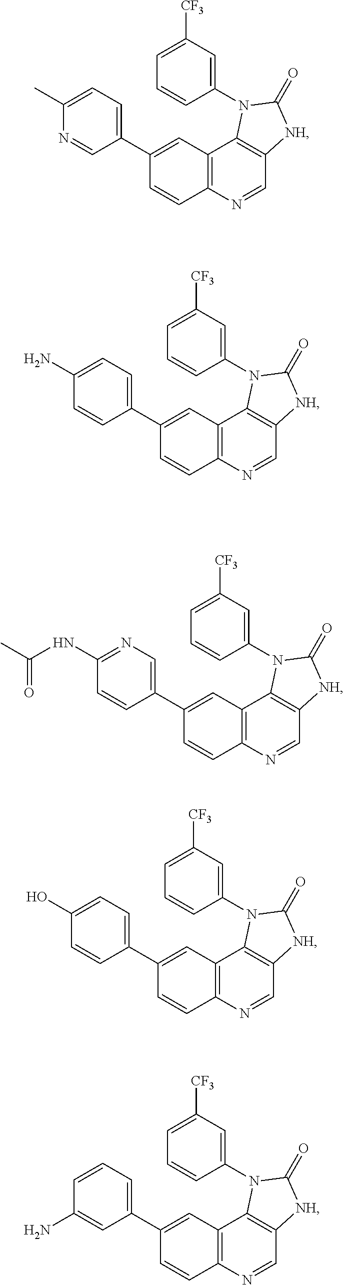 [SMILES: Cc1ccc(-c2ccc3ncc4[nH]c(=O)n(-c5cccc(C(F)(F)F)c5)c4c3c2)cn1 ; Nc1ccc(-c2ccc3ncc4[nH]c(=O)n(-c5cccc(C(F)(F)F)c5)c4c3c2)cc1 ; CC(=O)Nc1ccc(-c2ccc3ncc4[nH]c(=O)n(-c5cccc(C(F)(F)F)c5)c4c3c2)cn1 ; O=c1[nH]c2cnc3ccc(-c4ccc(O)cc4)cc3c2n1-c1cccc(C(F)(F)F)c1 ; Nc1cccc(-c2ccc3ncc4[nH]c(=O)n(-c5cccc(C(F)(F)F)c5)c4c3c2)c1]

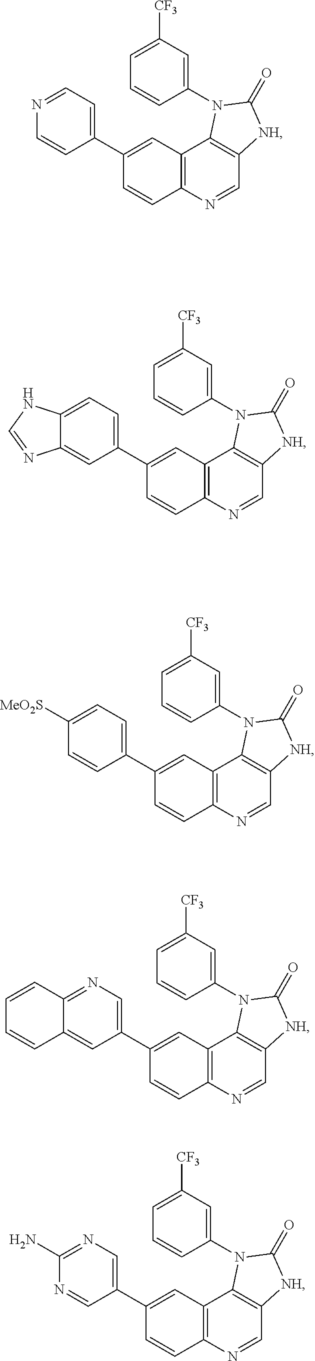 [SMILES: O=c1[nH]c2cnc3ccc(-c4ccncc4)cc3c2n1-c1cccc(C(F)(F)F)c1 ; O=c1[nH]c2cnc3ccc(-c4ccc5[nH]cnc5c4)cc3c2n1-c1cccc(C(F)(F)F)c1 ; CS(=O)(=O)c1ccc(-c2ccc3ncc4[nH]c(=O)n(-c5cccc(C(F)(F)F)c5)c4c3c2)cc1 ; O=c1[nH]c2cnc3ccc(-c4cnc5ccccc5c4)cc3c2n1-c1cccc(C(F)(F)F)c1 ; Nc1ncc(-c2ccc3ncc4[nH]c(=O)n(-c5cccc(C(F)(F)F)c5)c4c3c2)cn1]

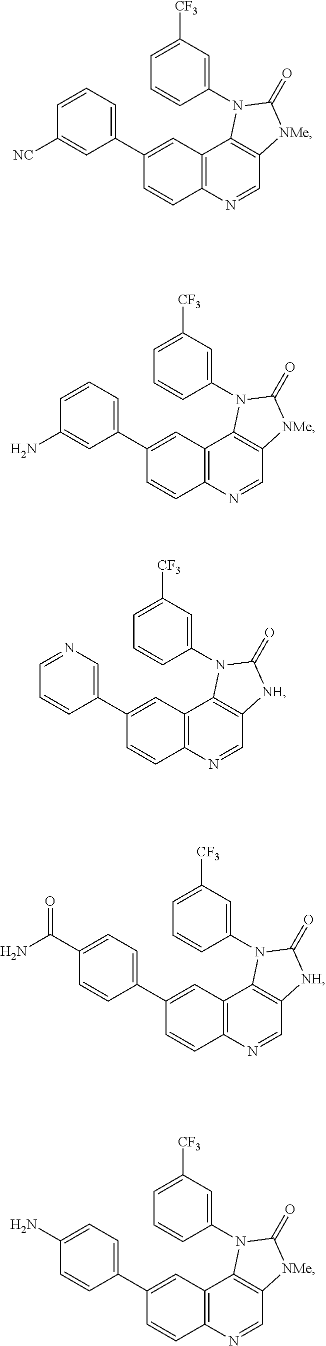 [SMILES: Cn1c(=O)n(-c2cccc(C(F)(F)F)c2)c2c3cc(-c4cccc(C#N)c4)ccc3ncc21 ; Cn1c(=O)n(-c2cccc(C(F)(F)F)c2)c2c3cc(-c4cccc(N)c4)ccc3ncc21 ; O=c1[nH]c2cnc3ccc(-c4cccnc4)cc3c2n1-c1cccc(C(F)(F)F)c1 ; NC(=O)c1ccc(-c2ccc3ncc4[nH]c(=O)n(-c5cccc(C(F)(F)F)c5)c4c3c2)cc1 ; Cn1c(=O)n(-c2cccc(C(F)(F)F)c2)c2c3cc(-c4ccc(N)cc4)ccc3ncc21]

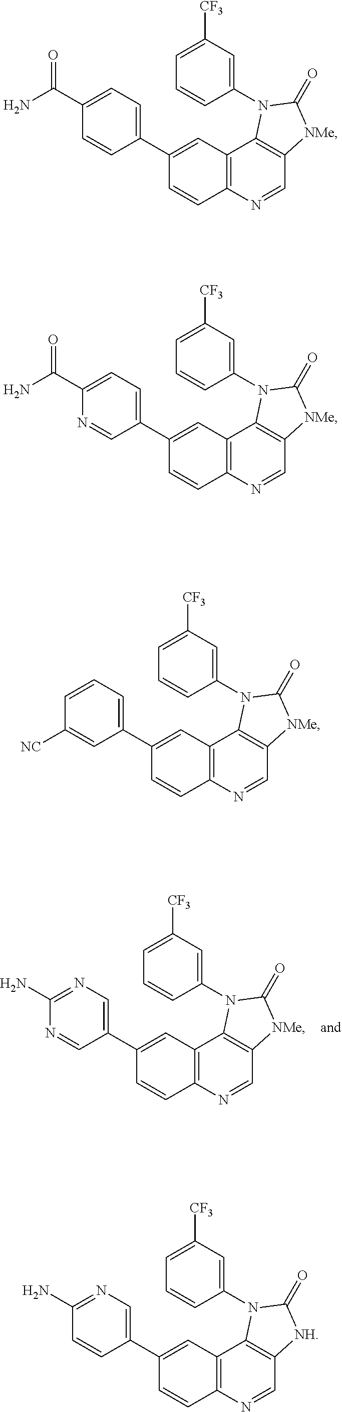

In certain embodiments, the second compound that is administered is selected from elesclomol, NSC174938, NVP-AUY922, Maduramicin, Narasin, Alvespimycin, Omacetaxine, Thiram, Zinc pyrithione, Phanquinone, Bortezomib, Salinomycin sodium, Monensin sodium, Dipyrithione, Dicyclopentamethylene-thiuram disulfide, YM155, Withaferin A, Adriamycin, Romidepsin, AZD-1152-HQPA, CAY10581, Plicamycin, CUDC-101, Auranofin, Trametinib, GSK-458, Afatinib, and Panobinostat. In certain preferred embodiments, elesclomol, the compound is NSC174938, NVP-AUY922, Maduramicin, and Narasin.

In accordance with an embodiment of the invention, the compound is administered in the form of a pharmaceutical composition comprising the compound and a pharmaceutically acceptable carrier.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

Suitable carriers and their formulations are further described in A. R. Gennaro, ed., Remington: The Science and Practice of Pharmacy (19th ed.), Mack Publishing Company, Easton, Pa. (1995).

The compound of the invention or a composition thereof can potentially be administered as a pharmaceutically acceptable acid-addition, base neutralized or addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, ammonium hydroxide, potassium hydroxide, and organic bases, such as mono-, di-, trialkyl, and aryl amines and substituted ethanolamines. The conversion to a salt is accomplished by treatment of the base compound with at least a stoichiometric amount of an appropriate acid. Typically, the free base is dissolved in an inert organic solvent such as diethyl ether, ethyl acetate, chloroform, ethanol, methanol, and the like, and the acid is added in a similar solvent. The mixture is maintained at a suitable temperature (e.g., between 0° C. and 50° C.). The resulting salt precipitates spontaneously or can be brought out of solution with a less polar solvent.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

The Plasmodium parasite can be any suitable Plasmodium parasite. Non-limiting examples of suitable Plasmodium parasites include Plasmodium falciparum, Plasmodium vivax, Plasmodium malariae, Plasmodium ovale, and Plasmodium knowlesi. In a preferred embodiment, the Plasmodium parasite is Plasmodium falciparum.

In an embodiment, the Plasmodium parasite is a Plasmodium gametocyte.

In embodiments, the Plasmodium gametocyte is a mature stage II-V gametocyte. In a preferred embodiment, the Plasmodium gametocyte is a stage III-V gametocyte, e.g., a mature stage III-V gametocyte. In another preferred embodiment, the Plasmodium gametocyte is a mature stage V gametocyte.

In certain preferred embodiments, the compound effectively kills Plasmodium gametocytes.

In embodiments, the Plasmodium parasite is a drug-resistant strain. Examples of drug-resistant strains of Plasmodium are described in Kun, J. F. J. et al., Antimicrob Agents Chemother., 1999 September; 43(9): 2205-2208, and references cited therein.

In embodiments, the Plasmodium parasite is in an asexual stage. For example, the Plasmodium parasite can be a sporozoite, a liver stage parasite, a merozoite, an asexual erythrocyte-stage parasite, a zygote, an ookinete, or an oocyst.

The amount or dose of a compound of the invention or a salt thereof, or a composition thereof should be sufficient to affect a therapeutic or prophylactic response in the mammal. The appropriate dose will depend upon several factors. For instance, the dose also will be determined by the existence, nature and extent of any adverse side effects that might accompany the administration of a particular compound or salt. Ultimately, the attending physician will decide the dosage of the compound of the present invention with which to treat each individual patient, taking into consideration a variety of factors, such as age, body weight, general health, diet, sex, compound or salt to be administered, route of administration, and the severity of the condition being treated. By way of example and not intending to limit the invention, the dose of the compound(s) described herein can be about 0.1 mg to about 1 g daily, for example, about 5 mg to about 500 mg daily. Further examples of doses include but are not limited to: 0.1 mg, 0.15 mg, 0.2 mg, 0.25 mg, 0.5 mg, 0.6 mg, 0.75 mg, 1 mg, 1.5 mg, 2 mg, 3 mg, 4 mg, 5 mg, 6 mg, 7 mg, 8 mg, 9 mg, 10 mg, 12 mg, 15 mg, 17 mg, 20 mg, 25 mg, 30 mg, 35 mg, 40 mg, 45 mg, 50 mg, 55 mg, 60 mg, 70 mg, 75 mg, 80 mg, 85 mg, 90 mg, 95 mg, 100 mg, 125 mg, 140 mg, 150 mg, 175 mg, 200 mg, 225 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg/kg body weight per day.

In certain embodiments, the method further comprises administering to the mammal at least one additional antimalarial compound. Any suitable antimalarial compound can be used, many of which are well known in the art. Non-limiting examples of suitable antimalarial compounds include primaquine, bulaquine, artemisinin and derivatives thereof, chloroquine, mefloquine, amodiaquine, piperaquine, pyronaridine, atovaquone, tafenoquine, methylene blue, trioxaquines, endoperoxides such as OZ 439 and OZ 277, decoquinate, 9-anilinoacridines, HIV-protease inhibitors, and natural products such as neem, epoxomicin, harmonine, and riboflavin.

The following examples further illustrate the invention but, of course, should not be construed as in any way limiting its scope.

Materials and Methods

Cell culture. Asexual parasites of P. falciparum strain 3D7 were cultured as described previously [55]. Stage III-V gametocytes were selected and enriched with 3-day treatment with 50 mM N-acetylglucosamine (NAG) and the following Percoll density gradient centrifugation after gametocyte production [10]. Gametocytes of HB3 and Dd2 strains were produced and then set up for assay in a similar process. HepG2 cells (ATCC, cat. no. 77400) were cultured in 175-cm² tissue culture flasks with 30 ml growth medium at 37° C. in a 5% CO₂ humidified atmosphere. Growth medium was made with Dulbecco's Modified Eagle Medium with 10% fetal bovine serum (FBS). Growth medium was replaced every other day and cells were passed at 75% confluence.

Compound library and gametocyte assay screen. The approved drug library was collected with 4,265 compounds from traditional chemical suppliers, specialty collections, pharmacies and custom synthesis [12] that included 49% drugs approved for human or animal use by the US Food and Drug Administration (FDA), 23% approved in Canada/UK/EU/Japan, and the remaining 28% either in clinical trials or research tool compounds. The Malaria Box contained 400 drugs or tool compounds with the confirmed activities on blood-staged P. falciparum and assessed cytotoxicity against mammalian cells [39, 56]. The MIPE library was an internal collection of 550 kinase inhibitors, which contain approved drugs and drug candidates in preclinical and clinical stages [14]. Compounds from all libraries were obtained as powder samples and dissolved in DMSO as 10 mM stock solutions, except several hundreds from the approved drug library that were prepared as 4.47 mM stock solutions due to solubility limitations.

Compound screening experiments were performed as previously described [11]. Briefly, 2.5 μl/well incomplete medium was dispensed into each well of 1,536-well plates using the Multidrop Combi followed by 23 nl compound transferring using the NX-TR Pintool (WAKO Scientific Solutions, San Diego, Calif.). Then, 2.5 μl/well of gametocytes was dispensed with a seeding density of 20,000 cells/well using the Multidrop Combi. The assay plates were incubated for 72 h at 37° C. with 5% CO₂. After addition of 5 μl/well of 2× AlamarBlue dye (Life Technologies, cat. no. DAL1100), the plates were incubated for 24 h at 37° C. with 5% CO₂ and then were read in a fluorescence detection mode (Ex=525 nm, Em=598 nm) on a ViewLux plate reader (PerkinElmer).

Small molecule pull-down. Affinity matrix: To make a bead-connected affinity probe of Torin 2, a tetraethylene glycol linker was attached to 1-(piperazin-1-yl)propan-1-one of HWW030 and then coupled to Affi-Gel 10 resin (Bio-Rad Laboratories, cat. no. 153-6046) under mild basic conditions to afford Torin 2 matrix (T2M). See detailed version in Example 7. Torin 1 was similarly immobilized to resin and used as a negative control (T1M). The resultant affinities probes were incubated with gametocyte lysates, the bound proteins were eluted from resin by boiling in SDS-PAGE sample loading buffer. The eluted fractions were separated by SDS-PAGE and visualized by silver staining. RBC infected with gametocytes (3D7 strain: Stage III-V) were washed 3 times with PBS and then lysed by 0.05% saponin treatment in PBS for 5 min at room temperature. The prepared gametocytes were washed 3 times with PBS and frozen at −80° C. The affinity precipitation experiment was processed as previously described [33, 57]. The frozen samples were lysed with homogenization buffer (60 mM glycerophosphate, 15 mM p-nitrophenyl phosphate, 25 mM MOPS (pH 7.2), 15 mM EGTA, 15 mM MgCl2, 1 mM DTT, protease inhibitors (Roche Diagnostics, cat. no. 11836170001), and 0.5% Nonidet P-40). Cell lysates were centrifuged at 16,000×g for 20 min at 4° C., and the supernatant was collected. Protein concentration in the supernatant was determined by using a BCA protein assay kit (Pierce Chemical, cat. no. 23225). The lysate (0.5 mg) was then added to the packed affinity matrix, and bead buffer (50 mM Tris HCl (pH 7.4), 5 mM NaF, 250 mM NaCl, 5 mM EDTA, 5 mM EGTA, protease inhibitors, and 0.1% Nonidet P-40) was added to a final volume of 1 ml. After rotating at 4° C. for 2 h, the mixture was centrifuged at 16,000×g for 2 min at 4° C., and the supernatant was removed. The affinity matrix was then washed (six times) with cold bead buffer and eluted by boiling with SDS-PAGE sample loading buffer at 95° C. for 5 min. Supernatants were separated on a 10% Bis-Tris gel (Life Technologies, cat. no. NP0315BOX) and visualized by silver staining using a Pierce Silver Stain Kit for Mass Spectrometry (Pierce Chemical, cat. no. 24600).

DARTS (drug affinity responsive target stability). The 3D7 gametocytes were lysed with M-PER supplemented with protease and phosphatase inhibitors as previously described [34]. After centrifugation at 16,000×g for 20 min, protein concentration in the supernatant was quantified and 2 μg/μl proteins were treated with 600 nM of Torin 2 or 600 nM of Torin 1 for 2 h at room temperature. The samples were treated with 46 μg/ml pronase (Sigma-Aldrich, cat. no. P6911) for 30 min at room temperature. The digestion was stopped by adding the SDS-PAGE sample loading buffer and boiled at 70° C. for 10 min. The samples were separated on a 10% Bis-Tris gel and visualized by silver staining.

Malaria Mouse Model. Plasmodium berghei ANKA (Pb) parasites were maintained by serial passage by intraperitoneal (i.p.) injection in outbred mice. Two days before feeding, female mice were infected i.p. with 200-400 μl whole blood from a Pb-infected mouse with >10% parasitemia. On the day of feeding, the mice were checked for exflagellation and injected intravenously (i.v.) with drug vehicle alone (10% N-methylpyrrolidnone, 40% PEG 400 in water), or (a) 2-4 mg/kg Torin 2 (one or two doses), (b) 8 mg/kg NVP-AUY922 (two doses), or (c) 8 mg/kg Alvespimycin (two doses). Two hours post treatment, mice were anesthetized and Anopheles stephensi mosquitoes were allowed to feed on infected mice for 15 minutes. Parasitemia, gametocytemia, and presence of exflagellation were examined as described previously [58]. Mosquitoes were maintained on 5% (w/v) glucose at 19° C. and 80% relative humidity. At day 10 post feeding, mosquito midguts were dissected and transmission was measured by staining mosquito midguts with 0.2% mercurochrome and counting the numbers of oocysts per midgut.

Data analysis. The primary screen data was analyzed using customized software developed internally [59]. IC₅₀ values were calculated using the Prism software (Graphpad Software, Inc. San Diego, Calif.). Data were presented as means±SEM with n=3 independent experiments.

General materials and methods for chemical synthesis. All commercially available reagents, compounds, and solvents were purchased and used without further purification. 9-Bromo-1-(3-(trifluoromethyl)phenyl)benzo[h][1,6]naphthyridin-2(1H)-one was prepared according to the method described by Liu and coworkers (Liu, Q. et. al., J. Med. Chem., 2011, 1473-1480). Column chromatography on silica gel was performed on Biotage KPSil pre-packed cartridges using the Biotage SP-1 automated chromatography system. Reverse phase column chromatography was performed on RediSep preparative C-18 column using the Teledyne ISCO combiflash Rf system. ¹H spectra were recorded using an Inova 400 MHz spectrometer (Varian). Samples were analyzed on an Agilent 1200 series LC/MS. Method A used an Enomenex Kinetex 1.7 micron column and a flow rate of 1.1 mL/min. The mobile phase was a mixture of acetonitrile and H₂O each containing 0.05% trifluoroacetic acid. A gradient of 4% to 100% acetonitrile over 4 minutes was used during analytical analysis. Method B used a Zorbax™ Eclipse XDB-C18 reverse phase (5 micron, 4.6×150 mm) column and a flow rate of 1.1 mL/min. The mobile phase was a mixture of acetonitrile and H₂O each containing 0.05% trifluoroacetic acid. A gradient of 5% to 100% acetonitrile over 8 minutes was used during analytical analysis.

### Example 1

This example describes an assay for the identification of gametocytocidal compound in accordance with an embodiment of the invention.

P. falciparum strain 3D7 gametocytes were screened against 5,215 compounds at four concentrations ranging from 0.37 to 46 μM using an alamarBlue viability assay [10, 11]. These compounds include 4,265 approved human or animal drugs [12], 400 from the Malaria Box library that are active against P. falciparum strain 3D7 asexual parasites in vitro [13], and 550 from an internal collection of kinase inhibitors [14]. A total of 27 novel active gametocytocidal compounds were identified and confirmed with IC₅₀ values ≦1 μM against gametocytes. Among these confirmed compounds, 21 had more than 10-fold selectivity against gametocytes over the mammalian cell line HepG2. The gametocial activity is set forth in Table 1, and the cytotoxicity against the mammalian HepG2 cell line is set forth in Table 2. NSC174938, Torin 2, NVP-AUY922, maduramicin, and narasin were the most potent compounds against gametocytes with IC₅₀ values ranging from 3 to 50 nM (Table 1). Additionally, PQ (primaquine) and 7 other compounds with known gametocytocidal activity were present in the compound collection and were all identified in the screen (Table 1), validating the effectiveness of this screening method.

TABLE 1

| | Gametocyte IC₅₀ | | |
|---|---|---|---|
| Compound Name | (μM) | Function class | Primary activity |
| NSC174938 | 0.003 | Tyrosyl-DNA | Anticancer |
| | | phosphodiesterase | |
| Elesclomol | 0.006 | Heat Shock Protein 70 | Anticancer |
| | | (HSP70) Inhibitor | |
| Torin 2*† | 0.008 | mTORC1 Inhibitor | Anticancer |
| Carfilzomib*† | 0.012 | Proteasome inhibitor | Anticancer |
| Dactinomycin*† | 0.015 | Transcription inhibitor | Anticancer, |
| | | | antibacterial |
| NVP-AUY922 | 0.047 | Heat Shock Protein 90 | Anticancer |
| | | (HSP90) Inhibitor | |
| Maduramicin | 0.047 | Ionophore | Antiprotozoal |
| Narasin* | 0.050 | Ionophore | Antiprotozoal, |
| | | | antibacterial |
| Artesunate*† | 0.059 | Alkylation of heme | Amebicides, |
| | | | Antimalarials |
| Artemether*† | 0.073 | Alkylation of heme | Antimalarial |
| Alvespimycin | 0.074 | Heat Shock Protein 90 | Anticancer |
| | | (hsp90) Inhibitor | |
| Artenimol (DHA) *† | 0.077 | Alkylation of heme | Antimalarials |
| Omacetaxine | 0.083 | Protein translation | Anticancer |
| | | inhibitor | |
| Thiram* | 0.083 | Metabolic poisons | Antifungal |
| Zinc pyrithione* | 0.093 | Copper import and | Antifungal |
| | | iron-sulphur proteins | |
| Phanquinone* | 0.109 | S-adenosylhomocysteine | Antibacterial, |
| | | hydrolase | antimalarial |
| Bortezomib* | 0.118 | Proteasome Inhibitor | Anticancer |
| Artemisinin*† | 0.148 | Alkylation of heme | Antimalarials |
| Salinomycin sodium* | 0.194 | Ionophore | Antibacterial, |
| | | | antiprotozoal |
| Monensin sodium* | 0.254 | Ionophore | Antimalarial, |
| | | | antiprotozoal |
| Dipyrithione | 0.263 | Membrane transport | Antibacterial, |
| | | inhibitor | antifungal |
| Dicyclopentamethylene- | 0. 274 | Monoglyceride lipase | Other |
| thiuram disulfide* | | (MGL) inhibitor | |
| Methylene blue*† | 0.307 | Monoamine oxidase | Antimalaria, |
| | | inhibitor | Anticancer |
| Quinine hemisulfate*† | 0.345 | Hemozoin | Antimalarial, |
| | | biocrystallization | analgesic, |
| | | inhibitor | antiinflammatory |
| YM155 | 0.372 | Survivin inhibitor | Anticancer |
| Withaferin A | 0.372 | NF-kappaB Activation | Anticancer |
| | | Inhibitor | |
| Adriamycin* | 0.526 | DNA synthesis inhibitor | Anticancer |
| Romidepsin | 0.637 | Histone deacetylase | Anticancer |
| | | (HDAC) inhibitor | |
| AZD-1152-HQPA | 0.743 | Aurora kinase inhibitor | Anticancer |
| CAY10581 | 0.743 | Indoleamine 2,3- | Anticancer |
| | | dioxygenase inhibitor | |
| Mefloquine*† | 0.833 | Heme polymerase | Antimalarial, |
| | | inhibitor | antiinflammatory |
| Plicamycin* | 0.833 | RNA synthesis inhibitor | Antibiotics, |
| | | | anticancer |
| CUDC-101 | 0.833 | Multi target Inhibitor of | Anticancer |
| | | HDA), EGFR/ErbB1, | |
| | | and HER2/neu or ErbB2 | |
| Auranofin* | 0.935 | Mitochondrial | Antirheumatic, |
| | | thioredoxin reductase | antiinflammatory |
| | | (TrxR) inhibitor | |
| Trametinib | 0.935 | Mitogen-activated | Anticancer |
| | | protein kinase kinase | |
| | | (MEK MAPK/ERK | |
| | | kinase) inhibitor | |
| GSK-458 | 0.935 | PI3K inhibitor | Anticancer |
| Afatinib | 0.935 | Dual receptor tyrosine | Anticancer |
| | | kinase (RTK) inhibitor | |
| Panobinostat | 0.935 | Selective histone | Anticancer |
| | | deacetylase inhibitor | |
| | | (HDAC) | |
| Puromycin*† | 1.049 | Transcription inhibitor | Antibiotic, |
| | | | antibacterial |
| Primaquine*† | 1.262 | Not clear | Antimalarial |

Note:
mean IC₅₀, mean half-maximum inhibitory concentrations determined from at least 3 independent experiments against P. falciparum 3D7_gametocyte;
*indicates compounds with previously reported activities against asexual parasites.
†means compounds with previously reported activities against gametocytes.

TABLE 2

| Compound | IC₅₀ (μM) in HepG2 | % Max response |
|---|---|---|
| NSC174938 | Inactive | 13 |
| Torin 2 | 9.350 | −46 |
| Carfilzomib | 1.177 | −79 |
| Dactinomycin | Inactive | 1 |
| NVP-AUY922 | 0.148 | −88 |
| Maduramicin | 37.221 | −42 |
| Narasin | 27.041 | −98 |
| Artesunate | Inactive | 18 |
| Artemether | Inactive | 7 |
| Alvespimycin | 0.118 | −94 |
| Artenimol (DHA) | Inactive | 20 |
| Omacetaxine | Curve not complete | −27 |
| Thiram | Inactive | 23 |
| Zinc pyrithione | 2.148 | −99 |
| Phanquinone | Inactive | 23 |
| Bortezomib | 0.148 | −82 |
| Artemisinin | Inactive | 18 |
| Salinomycin sodium | 29.566 | −69 |
| Monensin sodium | Inactive | 40 |
| Dipyrithione | 10.765 | −93 |
| Dicyclopentamethylenethiram | Inactive | 0 |
| disulfide | | |
| Methylene blue | Inactive | −20 |
| Quinine hemisulfate | Inactive | 2 |
| YM155 | 4.686 | −92 |
| Withaferin A | 9.350 | −80 |
| Adriamycin | Inactive | −35 |
| Romidepsin | 0.074 | −98 |
| AZD-1152-HQPA | Inactive | 11 |
| CAY10581 | 17.062 | −86 |
| Mefloquine | 29.566 | −97 |
| Plicamycin | Inactive | 3 |
| CUDC-101 | 6.793 | −76 |
| Auranofin | 9.350 | −98 |
| Trametinib | Inactive | 14 |
| GSK-458 | Inactive | 33 |
| Afatinib | 18.655 | −100 |
| Panobinostat | 0.372 | −74 |
| Puromycin | 11.770 | −71 |
| Primaquine | 17.831 | −65 |

Note:
“Inactive”: no significant activity at the highest tested compound concentration (46 μM).

### Example 2

This example demonstrates the profiles of gametocytocidal compounds against drug resistant strains in accordance with an embodiment of the invention.

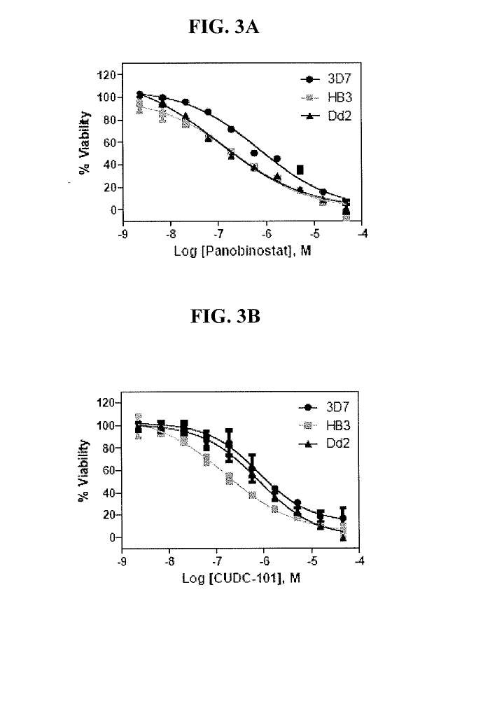

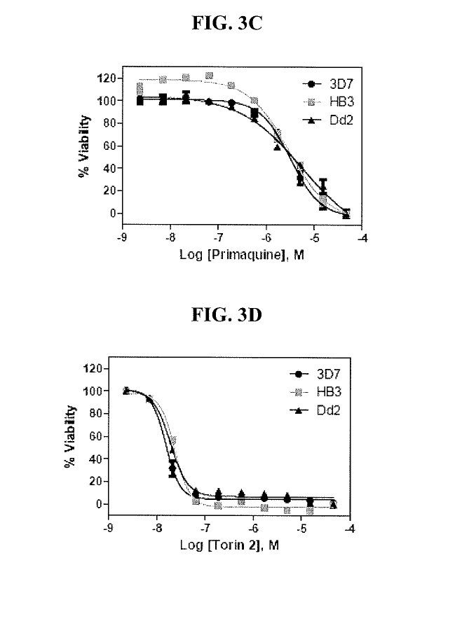

Drug resistance is also a critical challenge for malaria treatment and eradication that has not been examined in gametocytes, though it has been extensively studied for the asexual parasites [25,26]. To evaluate whether existing antimalarial agents and newly identified gametocytocidal compounds are effective against well characterized drug resistant strains, the gametocytocidal activities of 52 selected compounds, including 27 newly identified compounds and 25 known antimalarial agents, was determined against gametocytes of P. falciparum strains Dd2 and HB3 in the alamarBlue viability assay. In contrast to 3D7, asexual Dd2 parasites are resistant to chloroquine, mefloquine and pyrimethamine while asexual HB3 parasites are resistant to pyrimethamine but not chloroquine or mefloquine [27]. Most of 52 compounds showed 5-fold or less differences in potency between these two parasite strains compared to the drug sensitive stain 3D7. The results are set forth in Table 3. Compared to the drug sensitive stain 3D7, chloroquine's potency against Dd2 gametocytes was reduced 3.7-fold while it was 10-fold less potent against Dd2 asexual parasites [27]. Methylene blue was moderately more active against 3D7 gametocytes (IC₅₀=0.307 μM) than those of HB3 (IC₅₀=0.935 μM) and Dd2 (IC₅₀=0.526 μM) (SI Table 6). PQ showed similar potencies against gametocytes from these three strains with IC₅₀ values of 1.26, 0.68, and 1.08 μM against 3D7, HB3, and Dd2, respectively. The concentration-response curves of strain selective compounds panobinostat and CUDC-101 in comparison with strain nonselective compounds primaquine and Torin 2 are depicted in FIG. 3.

TABLE 3
Results of compound profiling against P. falciparum strains 3D7, HB3
and Dd2 gametocytes.

| | 3D7- | HB3- | Dd2- |
|---|---|---|---|
| Compound name | IC₅₀(μM) | IC₅₀(μM) | IC₅₀(μM) |
| Panobinostat | 0.9 | 0.148 | 0.118 |
| CUDC-101 | 0.8 | 0.152 | 0.429 |
| Carfilzomib | 0.0 | 0.003 | 0.002 |
| Torin-2 | 0.0 | 0.015 | 0.012 |
| Dactinomycin | 0.0 | 0.019 | 0.033 |
| Maduramicin ammonium | 0.0 | 0.012 | 0.037 |
| NVP-AUY922 | 0.0 | 0.042 | 0.047 |
| Narasin | 0.0 | 0.136 | 0.076 |
| Artesunate | 0.0 | 0.047 | 0.030 |
| Omacetaxine mepesuccinate | 0.0 | 0.017 | 0.037 |
| Lumefantrine | 0.0 | 0.033 | 0.013 |
| Mefloquine hydrochloride | 0.0 | 0.059 | 0.053 |
| Artemether | 0.0 | 0.047 | 0.053 |
| Alvespimycin | 0.0 | 0.235 | 0.094 |
| Artenimol | 0.0 | 0.059 | 0.053 |
| Thiram | 0.0 | 0.148 | 0.148 |
| Zinc pyrithione | 0.0 | 0.059 | 0.059 |
| Tetraethylthiuram disulfide | 0.0 | 0.148 | 0.296 |
| Disulfiram | 0.0 | 0.108 | 0.096 |
| Phanquinone | 0.1 | 0.037 | 0.053 |
| Salinomycin sodium | 0.1 | 0.372 | 0.296 |
| Bortezomib | 0.1 | 0.074 | 0.094 |
| Diphenyleneiodonium | 0.1 | 0.296 | 0.209 |
| Artemisinin | 0.1 | 0.074 | 0.061 |
| Salinomycin monosodium | 0.1 | 0.469 | 0.296 |
| Chloroquine diphosphate | 0.2 | 0.935 | 0.935 |
| Monensin sodium | 0.2 | 0.264 | 0.372 |
| Dipyrithione | 0.2 | 0.743 | 0.590 |
| Romidepsin | 0.2 | 0.148 | 0.187 |
| Dicyclopentamethylenethiuram | 0.2 | 0.743 | 0.743 |
| Methylene blue | 0.3 | 0.935 | 0.526 |
| Quinine hemisulfate | 0.3 | 0.235 | 0.083 |
| Withaferin A | 0.3 | 1.329 | 0.372 |
| YM155 | 0.372 | 0.304 | 0.526 |
| CyPPA | 0.469 | 0.743 | 0.590 |
| Adriamycin | 0.526 | 0.935 | 1.049 |
| 1,10-Phenanthroline | 0.743 | 1.177 | 1.321 |
| AZD-1152-HQPA | 0.743 | 1.482 | 1.482 |
| CAY10581 | 0.743 | 0.662 | 2.349 |
| Plicamycin | 0.833 | 2.957 | 2.349 |
| Auranofin | 0.935 | 1.049 | 1.177 |
| Ruthenium red | 0.935 | 0.264 | 0.526 |
| Afatinib | 0.935 | 4.176 | 2.957 |
| GSK-458 | 0.935 | 0.332 | 1.482 |
| Puromycin | 1.049 | 1.482 | 2.635 |
| Primaquine diphosphate | 1.262 | 0.679 | 1.077 |
| Clotrimazole | 1.866 | 1.482 | 1.482 |
| Pyronaridine | 1.866 | 2.635 | 6.619 |
| Calcimycin | 2.635 | 5.899 | 1.663 |
| Cyclosporin A | 3.317 | 0.935 | 1.866 |
| Torin-1 | 6.619 | 3.722 | 5.258 |
| Nizofenone | 23.485 | 14.818 | 16.626 |

Note:
Each compound was examined in 11 concentrations at a 1:3 dilution for three times against 3D7, HB3 or Dd2 gametocytes. Compounds showing more than 5-fold selectivity in two or three independent experiments against different strains were highlighted.

Interestingly, several of these newly identified gametocytocidal compounds exhibited similar or more favorable activities in these two asexual drug resistant strains compared to the drug sensitive 3D7 strain. For example, CUDC-101, a multi-target anticancer drug candidate [28] was 5.5-fold more potent against HB3 (IC₅₀=0.152 μM) compared to 3D7 (IC₅₀=0.833 μM) (FIG. 3). Additionally, panobinostat, a histone deacetylase inhibitor, was also 6.3 to 7.9 times more potent against Dd2 (IC₅₀=0.148 μM) and HB3 (IC₅₀=0.118 μM) compared to 3D7 (IC₅₀=0.935 μM) (FIG. 3). These results suggest that these newly identified gametocytocidal compounds could be also be effective against a range of asexual drug resistant isolates.

### Example 3

This example demonstrates activities of Torin 2 against gametocytes and asexual parasites in vitro in accordance with an embodiment of the invention.

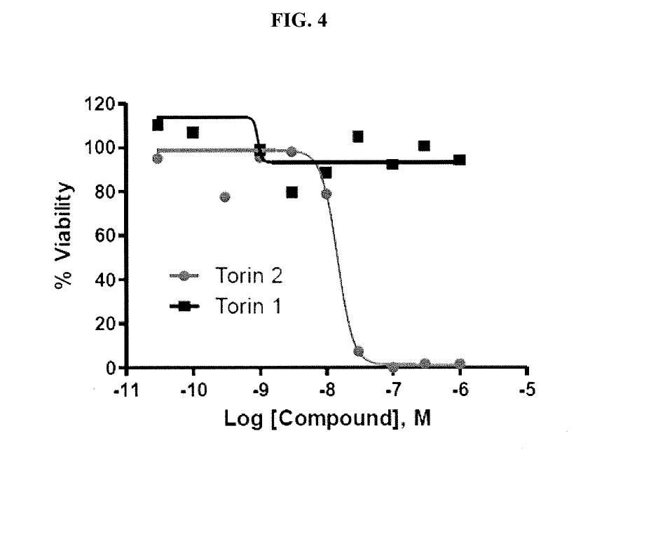

Torin 2, a known mTOR inhibitor [29, 30], was one of the most potent new gametocytocidal compounds (IC₅₀=8 nM). In contrast, its structural analog, Torin 1, was 200-fold less potent (IC₅₀=1.6 μM), regardless of their similar potencies on mTOR (IC₅₀ values of 5.4 and 2.1 nM, respectively) [29, 31]. The difference in gametocytocidal activity between the two compounds was confirmed using the traditional gametocyte viability assay, optical microscopy of Giemsa stained smears as depicted in FIG. 4. The 200-fold difference in potencies against P. falciparum gametocytes suggests that Torin 2 and Torin 1 may act on a different target or targets rather than mTOR, consistent with the lack of mTOR homolog in P. falciparum [32].

Because an ideal new antimalarial agent should have similar activities against both sexual and asexual parasites, IC₅₀ values of the two Torin compounds for asexual parasites in vitro were determined. In a viability assay using asexual parasites, Torin 2 exhibited an IC₅₀ of 2.75 nM, while Torin 1 had an IC₅₀ of 215 nM. Similarly, Torin 1 was 78 times less potent than Torin 2 against the asexual parasites and both compounds were slightly more potent against the asexual parasites compared to the gametocytes.

To assess the potential toxicity in mammalian cells, we examined both compounds in HepG2 cells. The results are set forth in Table 4. Torin 2 only exhibited partial cytotoxicity at the highest tested concentration (46 μM), indicative of greater than 1,000-fold selectivity against the parasites over the mammalian cells. Taken together, the results demonstrate the similar low nanomolar potencies of Torin 2 against both sexual and asexual stages of P. falciparum, as well as high selectivity against P. falciparum parasites over mammalian cells.

TABLE 4
Cytotoxicity of gametocytocidal compounds in mammalian HepG2 cell line.

| Compound | IC₅₀ (μM) in HepG2 cells | % Max response |
|---|---|---|
| NSC174938 | Inactive | 13 |
| Torin 2 | 9.350 | −46 |
| Carfilzomib | 1.177 | −79 |
| Dactinomycin | Inactive | 1 |
| NVP-AUY922 | 0.148 | −88 |
| Maduramicin | 37.221 | −42 |
| Narasin | 27.041 | −98 |
| Artesunate | Inactive | 18 |
| Artemether | Inactive | 7 |
| Alvespimycin | 0.118 | −94 |
| Artenimol (DHA) | Inactive | 20 |
| Omacetaxine | Curve not complete | −27 |
| Thiram | Inactive | 23 |
| Zinc pyrithione | 2.148 | −99 |
| Phanquinone | Inactive | 23 |
| Bortezomib | 0.148 | −82 |
| Artemisinin | Inactive | 18 |
| Salinomycin sodium | 29.566 | −69 |
| Monensin sodium | Inactive | 40 |
| Dipyrithione | 10.765 | −93 |
| Dicyclopentamethylenethiram | Inactive | 0 |
| disulfide | | |
| Methylene blue | Inactive | −20 |
| Quinine hemisulfate | Inactive | 2 |
| YM155 | 4.686 | −92 |
| Withaferin A | 9.350 | −80 |
| Adriamycin | Inactive | −35 |
| Romidepsin | 0.074 | −98 |
| AZD-1152-HQPA | Inactive | 11 |
| CAY10581 | 17.062 | −86 |
| Mefloquine | 29.566 | −97 |
| Plicamycin | Inactive | 3 |
| CUDC-101 | 6.793 | −76 |
| Auranofin | 9.350 | −98 |
| Trametinib | Inactive | 14 |
| GSK-458 | Inactive | 33 |
| Afatinib | 18.655 | −100 |
| Panobinostat | 0.372 | −74 |
| Puromycin | 11.770 | −71 |
| Primaquine | 17.831 | −65 |

Note:
“Inactive”: no significant activity at the highest tested compound concentration (46 μM).

### Example 4

This example demonstrates the efficacy of Torin 2 on gametocyte transmission from host to mosquitoes in a mouse model in accordance with an embodiment of the invention.

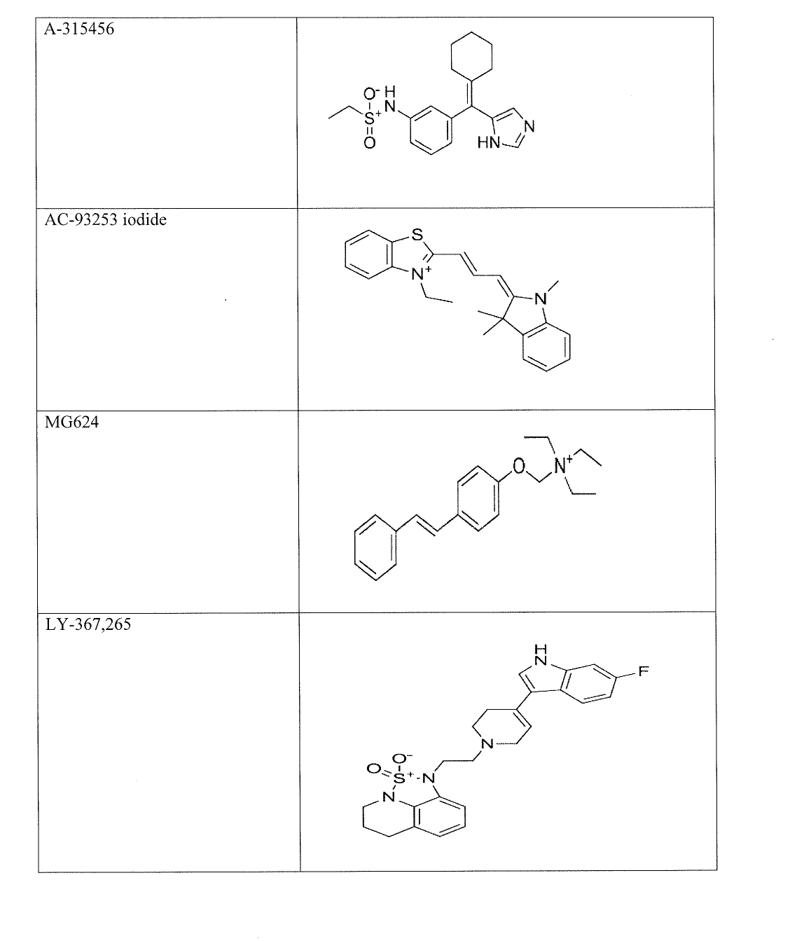

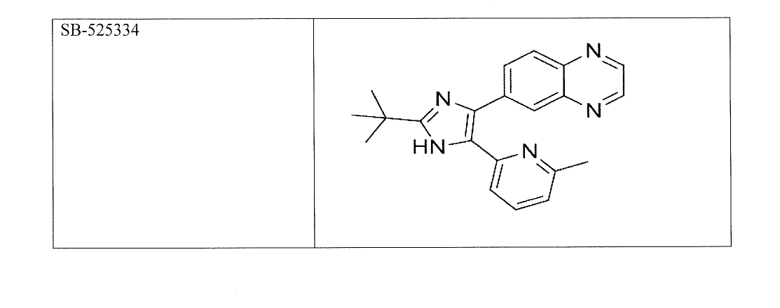

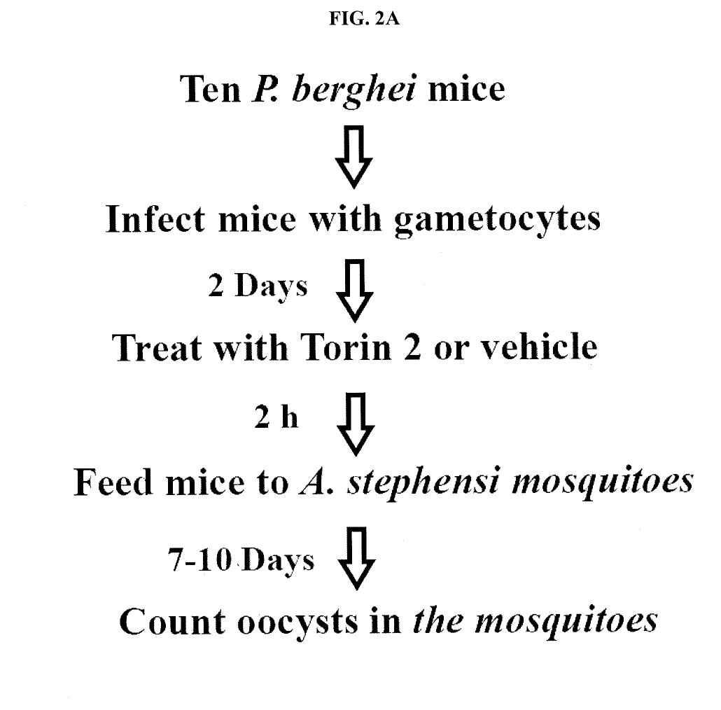

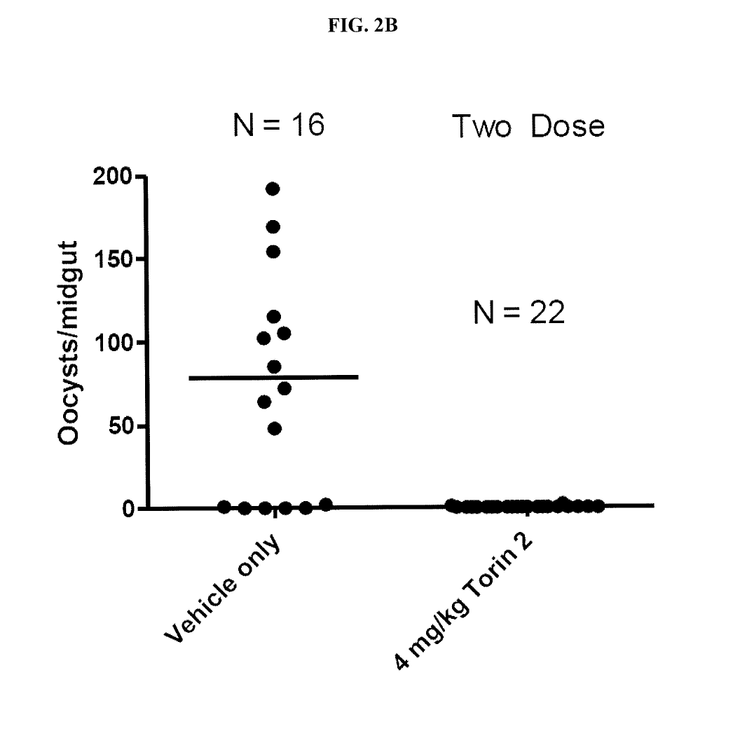

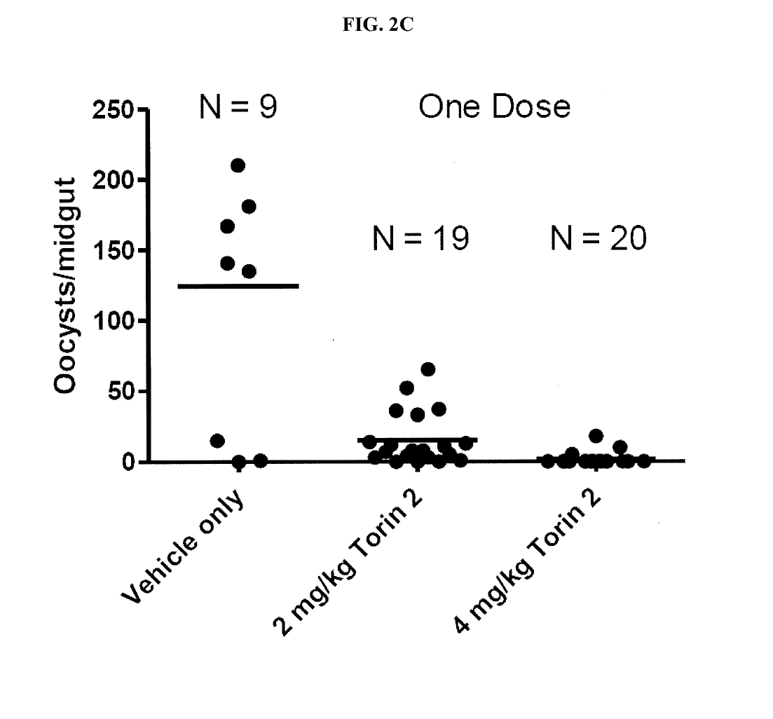

The transmission of Plasmodium berghei ANKA (Pb) from infected mice to Anopheles stephensi mosquitoes was examined to investigate the in vivo efficacy of Torin 2. After Pb infection, the mice were treated with either Torin 2 or a vehicle control (FIG. 2A), and mosquitoes were then allowed to feed on the infected mice. Oocyst production in these mosquitoes was used as an indication of malaria transmission. It was found that oocyst production in mosquitoes was completely blocked by the treatment of two 4 mg/kg doses of Torin 2 (FIG. 2B). To further evaluate the dose dependence, a single 2 or 4 mg/kg dose of Torin 2 was tested in the same mouse model (FIG. 2C). A single dose of 2 mg/kg of Torin 2 significantly reduced oocyst production, while a single 4 mg/kg dose almost completely eliminated it. These results clearly demonstrate the ability of Torin 2 to completely block gametocyte transmission from infected mice to mosquitoes.

### Example 5

This example demonstrates the identification of potential molecular targets of Torin 2 in accordance with an embodiment of the invention.

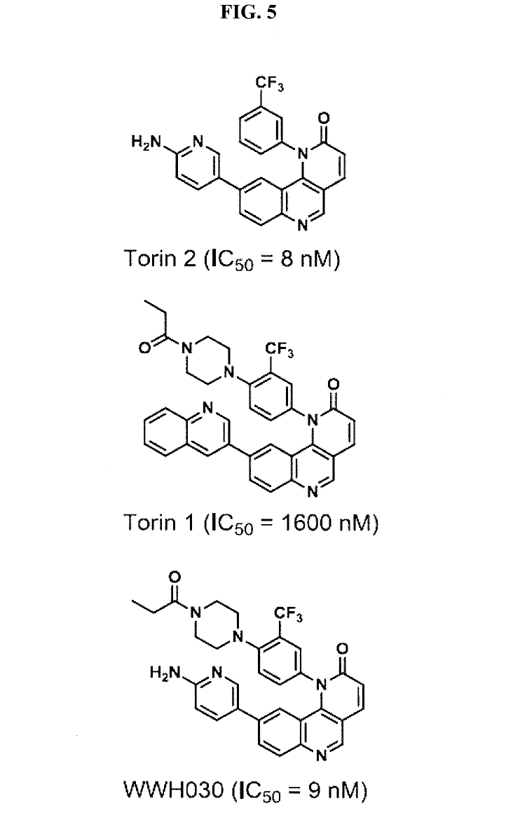

The lack of an mTOR homologue in P. falciparum [32] and significant difference in the potencies of Torin 1 and Torin 2 against the parasites suggest the presence of distinct targets in the parasites. It was hypothesized that Torin 2 selectively interacts with an unknown P. falciparum protein (or proteins) that has a weaker binding affinity to Torin 1. To develop a probe for an affinity based pull-down experiment, a Torin derivative, WWH030, was synthesized, and the importance of the ortho-piperazine-amide on the (trifluoromethyl)-benzene of Torin 2 for its gametocytocidal activity was determined. The structures of Torin 2, Torin 1, and WWH030 are depicted in FIG. 5. The new derivative had an IC₅₀ of 9 nM, similar to that of Torin 2 in the gametocyte assay. This result indicates that the ortho-piperazine-amide group on Torin 2 can be modified without a significant effect on its gametocytocidal activity. Therefore, T2M was synthesized as an affinity resin for the pull-down experiment for identification of Torin 2 interacting proteins in P. falciparum gametocyte lysates. The structure of T2M is shown as compound 10a in Example 7. A negative control resin, TIM, was similarly synthesized with a close analog of Torin 1, shown as compound 10b in Example 7.

The proteins precipitated from gametocyte lysate by T2M but not T1M were identified by mass spectrometric analysis [33]. The proteomics data revealed a total of 31 proteins selectively enriched by T2M. The results are set forth in Table 5. In parallel to the probe-protein precipitation experiment, a DARTS experiment [34] was also carried out to identify Torin 2 binding proteins by limited protease digestion of Torin 2-treated gametocyte lysates. Following treatment with either Torin 2 or the negative control Torin 1, gametocyte lysates were partially digested with pronase and size fractionated by SDS-PAGE. Four significant protein bands were enriched in the Torin 2-treated sample compared to the Torin 1-treated sample and analyzed by mass spectrometry. After comparing with the results from the affinity precipitation experiment, it was found that phosphoribosylpyrophosphate synthetase (PF3D7_1325100, ribose-phosphate diphosphokinase), aspartate carbamoyltransferase (PF3D7_1344800, ATCase), and a putative transporter (PF3D7_0914700) were identified by both experiments. Thus, these three gametocyte proteins are potential drug targets for Torin 2 and they will need to be further confirmed by enzyme assays and binding assays using recombinant P. falciparum proteins.

TABLE 5
Predicted Torin 2 interacting proteins in gametocytes by mass
spectrometry experiment.

| | ID of P. | Molecular | Unique |
|---|---|---|---|
| Protein | falciparum gene | weight | peptides |
| Phosphoribosylpyrophosphate | PF3D7_1325100 | 49 kDa | 4 |
| synthetase (Ribose-phosphate | | | |
| diphosphokinase) | | | |
| Flavoprotein subunit of succinate | PF3D7_1034400 | 71 kDa | 3 |
| dehydrogenase (SDHA) | | | |
| 6-phosphofructokinase (PFK11) | PF3D7_1128300 | 184 kDa | 3 |
| RNA-binding protein Nova-1, | PF3D7_1415300 | 38 kDa | 3 |
| putative | | | |
| Deoxyribodipyrimidine photolyase | PF3D7_0513600 | 129 kDa | 3 |
| (photoreactivating enzyme, DNA | | | |
| photolyase), putative | | | |
| Transporter, putative | PF3D7_0914700 | 58 kDa | 3 |
| Conserved Plasmodium protein, | PF3D7_1036900 | 193 kDa | 3 |
| unknown function | | | |
| Heat shock protein 60 (HSP60) | PF3D7_1015600 | 63 kDa | 2 |
| Nuclear protein localization protein | PF3D7_0507700 | 63 kDa | 2 |
| 4, putative (NPL4) | | | |
| Conserved Plasmodium protein, | PF3D7_1012900 | 44 kDa | 2 |
| unknown function | | | |
| Polyadenylate-binding protein, | PF3D7_1360900 | 45 kDa | 2 |
| putative | | | |
| Phosphatase, putative | PF3D7_1464600 | 170 kDa | 2 |
| Rhoptry-associated protein 2 | PF3D7_0501600 | 47 kDa | 2 |
| (RAP2) | | | |
| Deoxyribodipyrimidine photolyase | PF3D7_0513600 | 129 kDa | 2 |
| (photoreactivating enzyme, DNA | | | |
| photolyase), putative | | | |
| Multidrug resistance protein | PF3D7_0523000 | 162 kDa | 2 |
| (MDR1) | | | |
| Rifin (RIF) | PF3D7_0632700 | 42 kDa | 2 |
| Sin3 associated polypeptide | PF3D7_0711400 | 88 kDa | 2 |
| p18-like protein | | | |
| Merozoite surface protein 1 (MSP1) | PF3D7_0930300 | 196 kDa | 2 |
| Glycoprotease, putative | PF3D7_1030600 | 70 kDa | 2 |
| Conserved Plasmodium protein, | PF3D7_1142800 | 35 kDa | 2 |
| unknown function | | | |
| Plasmodium exported protein | PF3D7_1148700 | 44 kDa | 2 |
| (PHISTc), unknown function | | | |
| (GEXP12) | | | |
| Conserved Plasmodium protein, | PF3D7_1208900 | 167 kDa | 2 |
| unknown function | | | |
| DEAD/DEAH box ATP-dependent | PF3D7_1251500 | 83 kDa | 2 |
| RNA helicase, putative | | | |
| Aspartate carbamoyltransferase | PF3D7_1344800 | 43 kDa | 2 |
| (atcasE) | | | |
| Conserved Plasmodium protein, | PF3D7_1349600 | 36 kDa | 2 |
| unknown function | | | |
| Alanyl-tRNA synthetase, | PF3D7_1367700 | 165 kDa | 2 |
| Alanine--tRNA ligase (AlaRS) | | | |
| RNA binding protein, putative | PF3D7_1454000 | 59 kDa | 2 |
| Conserved Plasmodium membrane | PF3D7_1474600 | 46 kDa | 2 |
| protein, unknown function | | | |
| Plasmodium exported protein | PF3D7_0402100 | 68 kDa | 1 |
| (PHISTb), unknown function | | | |
| 60 S ribosomal protein L4, putative | PF3D7_0507100 | 46 kDa | 1 |
| ATP synthase subunit beta, | PF3D7_1235700 | 58 kDa | 1 |
| mitochondrial | | | |

Note:
Protein bands in both positive (Torin 2 pull-down) and negative (Torin 1 pull-down) samples were destained, reduced, and digested for mass spectrum. The mass spectrum data were analyzed by SEQUEST using PlasmoDB genomic database(http://www.plasmodb.org). Proteins with more than 1 unique peptide in positive samples and 0 unique peptide in negative samples were considered as Torin 2 selective interacting proteins.

### Example 6

This example demonstrates the synthesis of compounds in accordance with an embodiment of the invention.

General Procedure for the Synthesis of Compound 2:

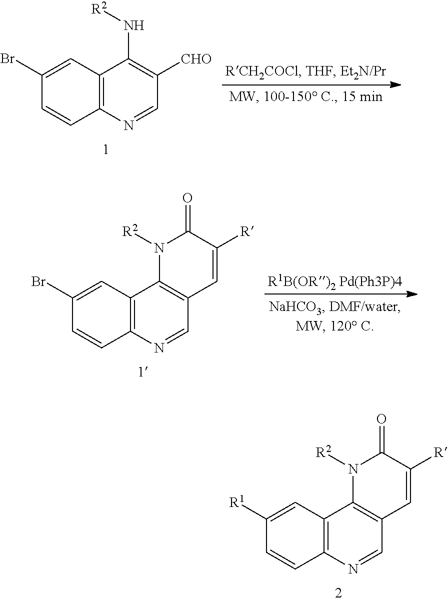

Aldehydes 1 were prepared using a reported procedure (J. Med. Chem. 2011, 54(5): 1473-1480). A solution of 1 (300 μmole) in 3 mL of THF were added 300 μL of Et₂NiPr and R′CH₂COCl (3000 μmole). The mixture was heated in a microwave between 100 to 150° C. for 15 min. The crude product was purified by column chromatography on silica gel using dichloromethane in methanol (0-20%) as eluent to give 1′. A mixture of 1′ (1.0 equiv), boronic acid or boronic acid pinacol ester (3.0 equiv), tetrakis(triphenylphosphine)palladium (0.05 equiv), DMF (1.5 mL) and saturated NaHCO₃ aqueous solution (0.5 mL) was charged in a microwave vial. Nitrogen was bubbled through the mixture for 3 min. The vial was capped and heated in a microwave at 120-150° C. for 15 min. The reaction mixture was filtered through a plug of Celite and the filtrate was purified by reverse phase column chromatography using acetonitrile (containing 0.1% TFA)/water (containing 0.1% TFA) as an eluent to give 2.

### 9-(2-Aminopyrimidin-5-yl)-1-(3-(trifluoromethyl)phenyl)benzo[h][1,6]naphthyridin-2(1H)-one

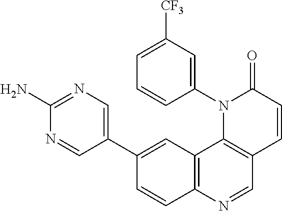 [SMILES: Nc1ncc(-c2ccc3ncc4ccc(=O)n(-c5cccc(C(F)(F)F)c5)c4c3c2)cn1]

¹H NMR (400 MHz, DMSO-d₆) δ 9.15 (s, 1H), 8.34 (d, J=9.5 Hz, 1H), 8.08-8.10 (m, 2H), 8.04-7.81 (m, 6H), 6.91-6.99 (m, 4H); LC/MS (Method A): (electrospray+ve), m/z 434.1 (MH)⁺, t_{R}=1.61 min, UV₂₅₄=98%.

### 9-(Pyrimidin-5-yl)-1-(3-(trifluoromethyl)phenyl)benzo[h][1,6]naphthyridin-2(1H)-one

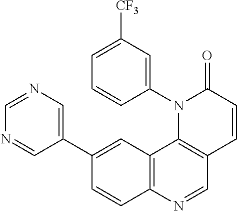 [SMILES: O=c1ccc2cnc3ccc(-c4cncnc4)cc3c2n1-c1cccc(C(F)(F)F)c1]

¹H NMR (400 MHz, DMSO-d₆) δ 9.24 (s, 1H), 9.17 (s, 1H), 8.55 (s, 2H), 8.37 (d, J=9.5 Hz, 1H), 8.24-8.10 (m, 3H), 8.05-7.98 (m, 1H), 7.95-7.80 (m, 2H), 7.05-6.95 (m, 2H); LC/MS (Method A): (electrospray+ve), m/z 419.1 (MH)⁺, t_{R}=1.84 min, UV₂₅₄=98%.

### 9-(Pyridin-3-yl)-1-(3-(trifluoromethyl)phenyl)benzo[h][1,6]naphthyridin-2(1H)-one

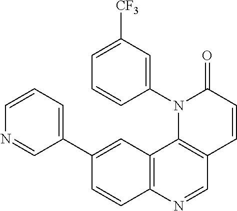 [SMILES: O=c1ccc2cnc3ccc(-c4cccnc4)cc3c2n1-c1cccc(C(F)(F)F)c1]

LC/MS (Method A): (electrospray+ve), m/z 418.1 (MH)⁺, t_{R}=1.51 min, UV₂₅₄=98%.

### 9-(Pyridin-4-yl)-1-(3-(trifluoromethyl)phenyl)benzo[h][1,6]naphthyridin-2(1H)-one

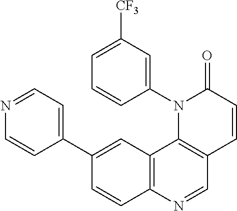 [SMILES: O=c1ccc2cnc3ccc(-c4ccncc4)cc3c2n1-c1cccc(C(F)(F)F)c1]

¹H NMR (400 MHz, DMSO-d₆) δ 9.22 (s, 1H), 8.59 (dd, J=4.8, 1.6 Hz, 1H), 8.43-8.33 (m, 2H), 8.23-7.80 (m, 6H), 7.57-7.41 (m, 21-1), 7.07-6.93 (m, 2H); LC/MS (Method A): (electrospray+ve), m/z 418.1 (MH)⁺, t_{R}=1.65 min, UV₂₅₄=100%.

### 9-Phenyl-1-(3-(trifluoromethyl)phenyl)benzo[h][1,6]naphthyridin-2(1H)-one

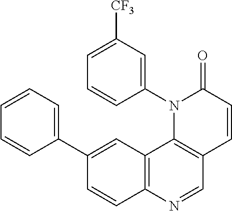 [SMILES: O=c1ccc2cnc3ccc(-c4ccccc4)cc3c2n1-c1cccc(C(F)(F)F)c1]

¹H NMR (400 MHz, DMSO-d₆) δ 9.19 (s, 1H), 8.35 (d, J=9.4 Hz, 1H), 8.20-8.09 (m, 2H), 8.09-8.00 (m, 2H), 7.88 (dd, J=8.4, 7.4 Hz, 1H), 7.84-7.76 (m, 1H), 7.42-7.32 (m, 3H), 7.15-7.05 (m, 3H), 6.96 (d, J=9.4 Hz, 1H); LC/MS (Method A): (electrospray+ve), m/z 417.1 (MH)⁺, t_{R}=2.39 min, UV₂₅₄=95%.

### 9-(4-Aminophenyl)-1-(3-(trifluoromethyl)phenyl)benzo[h][1,6]naphthyridin-2(1H)-one

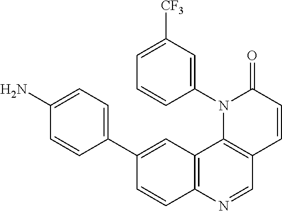 [SMILES: Nc1ccc(-c2ccc3ncc4ccc(=O)n(-c5cccc(C(F)(F)F)c5)c4c3c2)cc1]

LC/MS (Method A): (electrospray+ve), m/z 432.1 (MH)⁺, t_{R}=1.70 min, UV₂₅₄=95%.

### 9-(6-Amino-4-(trifluoromethyl)pyridin-3-yl)-1-(3-(trifluoromethyl)phenyl)benzo[h][1,6]naphthyridin-2(1H)-one

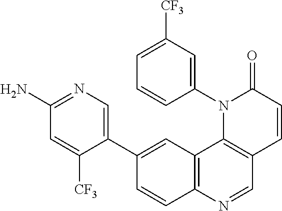 [SMILES: Nc1cc(C(F)(F)F)c(-c2ccc3ncc4ccc(=O)n(-c5cccc(C(F)(F)F)c5)c4c3c2)cn1]

LC/MS (Method B): (electrospray+ve), m/z 501.1 (MH)⁺, t_{R}=4.39 min, UV₂₅₄=95%.

### 9-(2-amino-4-methylpyrimidin-5-yl)-1-(3-(trifluoromethyl)phenyl)benzo[h][1,6]naphthyridin-2(1H)-one

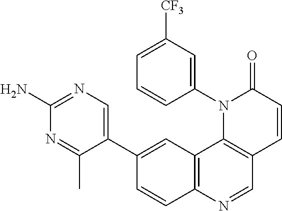 [SMILES: Cc1nc(N)ncc1-c1ccc2ncc3ccc(=O)n(-c4cccc(C(F)(F)F)c4)c3c2c1]

LC/MS (Method B): (electrospray+ve), m/z 448.1 (MH)⁺, t_{R}=3.84 min, UV₂₅₄=100%.

### N-(5-(2-Oxo-1-(3-(trifluoromethyl)phenyl)-1,2-dihydrobenzo[h][1,6]naphthyridin-9-yl)pyridin-2-yl)acetamide

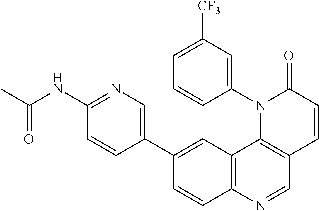 [SMILES: CC(=O)Nc1ccc(-c2ccc3ncc4ccc(=O)n(-c5cccc(C(F)(F)F)c5)c4c3c2)cn1]

LC/MS (Method B): (electrospray+ve), m/z 475.1 (MH)⁺, t_{R}=4.43 min, UV₂₅₄=98%.

### 9-(1H-Indazol-5-yl)-1-(3-(trifluoromethyl)phenyl)benzo[h][1,6]naphthyridin-2(1H)-one

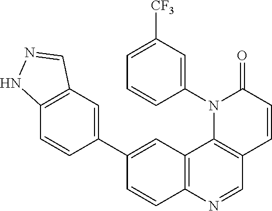 [SMILES: O=c1ccc2cnc3ccc(-c4ccc5[nH]ncc5c4)cc3c2n1-c1cccc(C(F)(F)F)c1]

LC/MS (Method B): (electrospray+ve), m/z 457.1 (MH)⁺, t_{R}=4.68 min, UV₂₅₄=95%.

### 9-(1H-Indol-5-yl)-1-(3-(trifluoromethyl)phenyl)benzo[h][1,6]naphthyridin-2(1H)-one

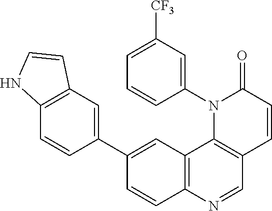 [SMILES: O=c1ccc2cnc3ccc(-c4ccc5[nH]ccc5c4)cc3c2n1-c1cccc(C(F)(F)F)c1]

LC/MS (Method B): (electrospray+ve), m/z 456.1 (MH)⁺, t_{R}=5.00 min, UV₂₅₄=95%.

### 9-(1H-Benzo[d]imidazol-6-yl)-1-(3-(trifluoromethyl)phenyl)benzo[h][1,6]naphthyridin-2(1H)-one

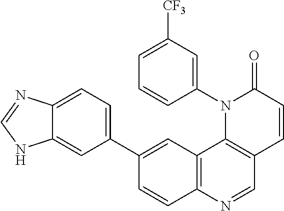 [SMILES: O=c1ccc2cnc3ccc(-c4ccc5nc[nH]c5c4)cc3c2n1-c1cccc(C(F)(F)F)c1]

LC/MS (Method B): (electrospray+ve), m/z 457.1 (MH)⁺, t_{R}=3.75 min, UV₂₅₄=90%.

### 9-(1H-Pyrazol-4-yl)-1-(3-(trifluoromethyl)phenyl)benzo[h][1,6]naphthyridin-2(1H)-one

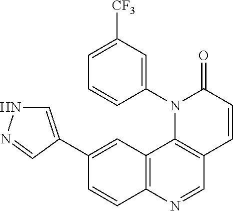 [SMILES: O=c1ccc2cnc3ccc(-c4cn[nH]c4)cc3c2n1-c1cccc(C(F)(F)F)c1]

LC/MS (Method B): (electrospray+ve), m/z 407.1 (MH)⁺, t_{R}=4.07 min, UV₂₅₄=100%.

### 9-(6-(Piperidin-1-yl)pyridin-3-yl)-1-(3-(trifluoromethyl)phenyl)benzo[h][1,6]naphthyridin-2(1H)-one

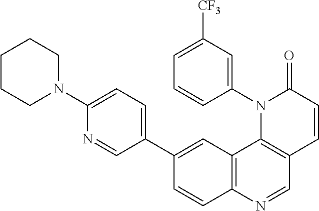 [SMILES: O=c1ccc2cnc3ccc(-c4ccc(N5CCCCC5)nc4)cc3c2n1-c1cccc(C(F)(F)F)c1]

¹H NMR (400 MHz, DMSO-d₆) δ 9.21 (s, 1H), 8.35 (d, J=9.5 Hz, 1H), 8.15-7.99 (m, 4H), 7.99-7.81 (m, 3H), 7.17 (dd, J=9.2, 2.5 Hz, 1H), 7.04-6.91 (m, 3H), 3.72-3.58 (m, 4H), 1.71-1.50 (m, 6H); LC/MS (Method B): (electrospray+ve), m/z 501.2 (MH)⁺, t_{R}=4.31 min, UV₂₅₄=95%.

### 9-(6-(Dimethylamino)pyridin-3-yl)-1-(3-(trifluoromethyl)phenyl)benzo[h][1,6]naphthyridin-2(1H)-one

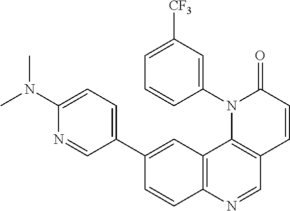 [SMILES: CN(C)c1ccc(-c2ccc3ncc4ccc(=O)n(-c5cccc(C(F)(F)F)c5)c4c3c2)cn1]

LC/MS (Method B): (electrospray+ve), m/z 562.1 (MH)⁺, t_{R}=4.79 min, UV₂₅₄=90%.

### N-(4-(2-Oxo-1-(3-(trifluoromethyl)phenyl)-1,2-dihydrobenzo[h][1,6]naphthyridin-9-yl)phenyl)methanesulfonamide

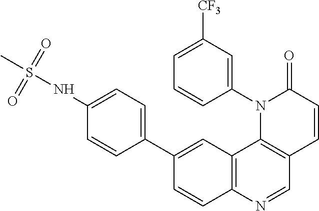 [SMILES: CS(=O)(=O)Nc1ccc(-c2ccc3ncc4ccc(=O)n(-c5cccc(C(F)(F)F)c5)c4c3c2)cc1]

¹H NMR (400 MHz, DMSO-d₆) δ 9.90 (s, 1H), 9.20 (s, 1H), 8.35 (d, J=9.5 Hz, 1H), 8.21-7.93 (m, 4H), 7.96-7.67 (m, 2H), 7.34-7.13 (m, 2H), 7.13-6.91 (m, 4H), 3.02 (s, 3H); LC/MS (Method B): (electrospray+ve), m/z 510.1 (MH)⁺, t_{R}=4.49 min, UV₂₅₄=90%.

### 9-(2-(4-Acetylpiperazin-1-yl)pyrimidin-5-yl)-1-(3-(trifluoromethyl)phenyl)benzo[h][1,6]naphthyridin-2(1H)-one

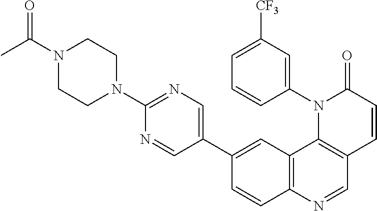 [SMILES: CC(=O)N1CCN(c2ncc(-c3ccc4ncc5ccc(=O)n(-c6cccc(C(F)(F)F)c6)c5c4c3)cn2)CC1]

¹H NMR (400 MHz, DMSO-d₆) δ 9.20 (s, 1H), 8.35 (d, J=9.5 Hz, 1H), 8.15-7.99 (m, 4H), 7.96-7.80 (m, 2H), 7.18 (dd, J=9.0, 2.6 Hz, 1H), 7.00-6.93 (m, 2H), 6.86 (d, J=9.0 Hz, 1H), 3.52-3.64 (m, 8H), 2.06 (s, 3H); LC/MS (Method B): (electrospray+ve), m/z 545.2 (MH)⁺, t_{R}=3.86 min, UV₂₅₄=98%.

### 9-(3-Aminophenyl)-1-(3-(trifluoromethyl)phenyl)benzo[h][1,6]naphthyridin-2(1H)-one

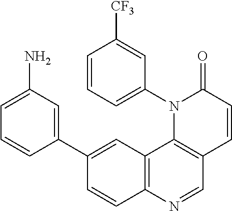 [SMILES: Nc1cccc(-c2ccc3ncc4ccc(=O)n(-c5cccc(C(F)(F)F)c5)c4c3c2)c1]

¹H NMR (400 MHz, DMSO-d₆) δ 9.12 (s, 1H), 8.31 (d, J=9.4 Hz, 1H), 8.07 (d, J=8.6 Hz, 2H), 8.04 (s, 1H), 7.90 (d, J=7.9 Hz, 1H), 7.87 (s, 1H), 7.79 (s, 1H), 7.03 (d, J=1.9 Hz, 1H), 6.94 (d, J=7.8 Hz, 1H), 6.93-6.90 (m, 1H), 6.52 (dd, J=7.8, 2.2 Hz, 1H), 6.44 (d, J=2.1 Hz, 1H), 6.04-5.99 (m, 1H), 5.04 (s, 2H); LC/MS (Method B): (electrospray+ve), m/z 432.1 (MH)⁺, t_{R}=4.045, UV₂₅₄=100%

### 9-(4-(Dimethylamino)phenyl)-1-(3-(trifluoromethyl)phenyl)benzo[h][1,6]naphthyridin-2(1H)-one

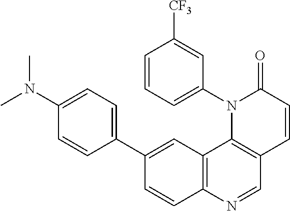 [SMILES: CN(C)c1ccc(-c2ccc3ncc4ccc(=O)n(-c5cccc(C(F)(F)F)c5)c4c3c2)cc1]

LC/MS (Method B): (electrospray+ve), m/z 460.1 (MH)⁺, t_{R}=4.780, UV₂₅₄=100%

### 1-(3-(Trifluoromethyl)phenyl)-9-vinylbenzo[h][1,6]naphthyridin-2(1H)-one

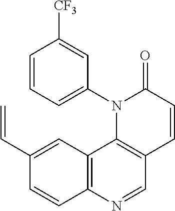 [SMILES: C=Cc1ccc2ncc3ccc(=O)n(-c4cccc(C(F)(F)F)c4)c3c2c1]

¹H NMR (400 MHz, DMSO-d₆) δ 9.12 (s, 1H), 8.31 (d, J=9.4 Hz, 1H), 8.06 (s, 1H), 8.04 (s, 1H), 8.00 (d, J=8.6 Hz, 1H), 7.90 (t, J=8.1 Hz, 1H), 7.82 (dd, J=8.5, 1.8 Hz, 2H), 6.93 (d, J=9.4 Hz, 1H), 6.58 (d, J=1.7 Hz, 1H), 6.25 (dd, J=17.6, 10.9 Hz, 1H), 5.35 (d, J=17.6 Hz, 1H), 5.19 (d, J=10.9 Hz, 1H); LC/MS (Method B): (electrospray+ve), m/z 367.1 (MH)⁺, t_{R}=4.837, UV₂₅₄=100%

### 4-(2-oxo-1-(3-(Trifluoromethyl)phenyl)-1,2-dihydrobenzo[h][1,6]naphthyridin-9-yl)benzamide

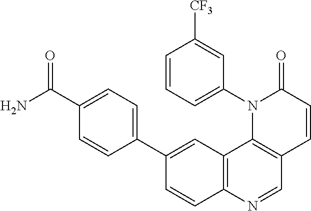 [SMILES: NC(=O)c1ccc(-c2ccc3ncc4ccc(=O)n(-c5cccc(C(F)(F)F)c5)c4c3c2)cc1]

¹H NMR (400 MHz, DMSO-d₆) δ 9.17 (s, 1H), 8.34 (d, J=9.5 Hz, 1H), 8.15 (d, J=2.0 Hz, 1H), 8.13 (d, J=8.7 Hz, 1H), 8.06 (dd, J=8.7, 1.9 Hz, 2H), 8.02 (s, 1H), 7.90-7.79 (m, 4H), 7.40 (s, 1H), 7.16 (d, J=8.4 Hz, 2H), 7.11 (d, J=1.9 Hz, 1H), 6.94 (d, J=9.4 Hz, 1H); LC/MS (Method B): (electrospray+ve), m/z 460.1 (MH)⁺, t_{R}=4.142, UV₂₅₄=100%

### 1,9-Bis(3-(trifluoromethyl)phenyl)benzo[h][1,6]naphthyridin-2(1H)-one

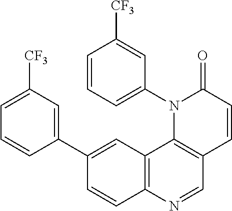 [SMILES: O=c1ccc2cnc3ccc(-c4cccc(C(F)(F)F)c4)cc3c2n1-c1cccc(C(F)(F)F)c1]

¹H NMR (400 MHz, DMSO-d₆) δ 9.20 (s, 1H), 8.34 (d, J=9.2 Hz, 1H), 8.16 (d, J=8.6 Hz, 1H), 8.09 (s, 1H), 8.06 (d, J=8.4 Hz, 1H), 7.96 (d, J=7.3 Hz, 1H), 7.87 (q, J=8.8, 8.4 Hz, 2H), 7.72 (d, J=7.8 Hz, 1H), 7.61 (t, J=7.8 Hz, 1H), 7.48 (d, J=8.0 Hz, 1H), 7.24 (s, 1H), 7.08 (s, 1H), 6.96 (s, 1H); LC/MS (Method B): (electrospray+ve), m/z 485.1 (MH)⁺, t_{R}=5.847, UV₂₅₄=88%

### 3-(2-Oxo-1-(3-(trifluoromethyl)phenyl)-1,2-dihydrobenzo[h][1,6]naphthyridin-9-yl)benzonitrile

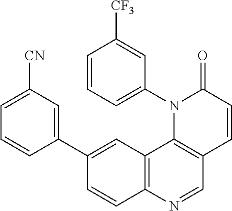 [SMILES: N#Cc1cccc(-c2ccc3ncc4ccc(=O)n(-c5cccc(C(F)(F)F)c5)c4c3c2)c1]

LC/MS (Method B): (electrospray+ve), m/z 442.1 (MH)⁺, t_{R}=5.148, UV₂₅₄=100%

### 1-(3-(Trifluoromethyl)phenyl)-9-(6-(trifluoromethyl)pyridin-3-yl)benzo[h][1,6]naphthyridin-2(1H)-one

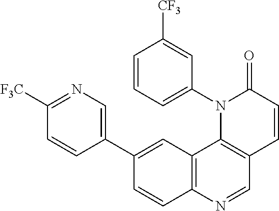 [SMILES: O=c1ccc2cnc3ccc(-c4ccc(C(F)(F)F)nc4)cc3c2n1-c1cccc(C(F)(F)F)c1]

LC/MS (Method B): (electrospray+ve), m/z 486.1 (MH)⁺, t_{R}=5.431, UV₂₅₄=100%

### 9-(4-(1H-Tetrazol-5-yl)phenyl)-1-(3-(trifluoromethyl)phenyl)benzo[h][1,6]naphthyridin-2(1H)-one

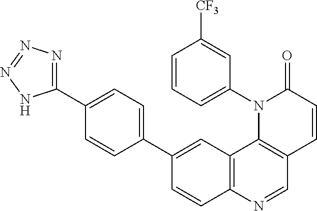 [SMILES: O=c1ccc2cnc3ccc(-c4ccc(-c5nnn[nH]5)cc4)cc3c2n1-c1cccc(C(F)(F)F)c1]

LC/MS (Method B): (electrospray+ve), m/z 485.1 (MH)⁺, t_{R}=4.470, UV₂₅₄=100%

### 9-(4-Chlorophenyl)-1-(3-(trifluoromethyl)phenyl)benzo[h][1,6]naphthyridin-2(1H)-one

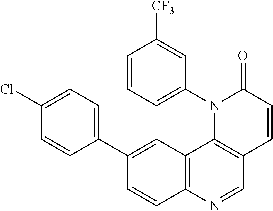 [SMILES: O=c1ccc2cnc3ccc(-c4ccc(Cl)cc4)cc3c2n1-c1cccc(C(F)(F)F)c1]

LC/MS (Method B): (electrospray+ve), m/z 451.0 (MH)⁺, t_{R}=5.762, UV₂₅₄=100%

### 9-(4-Methoxyphenyl)-1-(3-(trifluoromethyl)phenyl)benzo[h][1,6]naphthyridin-2(1H)-one

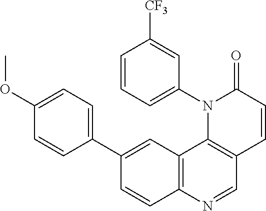 [SMILES: COc1ccc(-c2ccc3ncc4ccc(=O)n(-c5cccc(C(F)(F)F)c5)c4c3c2)cc1]

LC/MS (Method B): (electrospray+ve), m/z 447.1 (MH)⁺, t_{R}=5.306, UV₂₅₄=100%

### 3-(2-Oxo-1-(3-(trifluoromethyl)phenyl)-1,2-dihydrobenzo[h][1,6]naphthyridin-9-yl)benzamide

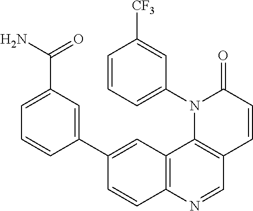 [SMILES: NC(=O)c1cccc(-c2ccc3ncc4ccc(=O)n(-c5cccc(C(F)(F)F)c5)c4c3c2)c1]

¹H NMR (400 MHz, DMSO-d₆) δ 9.17 (s, 1H), 8.34 (d, J=9.3 Hz, 1H), 8.14 (d, J=8.6 Hz, 1H), 8.10 (s, 1H), 8.09-8.05 (m, 1H), 8.00 (d, J=6.9 Hz, 2H), 7.87 (t, J=7.9 Hz, 1H), 7.80 (d, J=7.5 Hz, 3H), 7.41 (d, J=2.9 Hz, 1H), 7.38 (d, J=8.0 Hz, 1H), 7.05 (d, J=1.9 Hz, 1H), 6.98 (d, J=7.9 Hz, 1H), 6.94 (d, J=9.4 Hz, 1H); LC/MS (Method B): (electrospray+ve), m/z 459.7 (MH)⁺, t_{R}=4.196, UV₂₅₄=100%

### 9-(3-Acetylphenyl)-1-(3-(trifluoromethyl)phenyl)benzo[h][1,6]naphthyridin-2(1H)-one

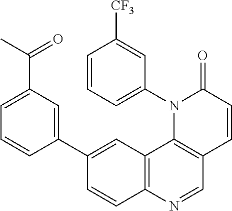 [SMILES: CC(=O)c1cccc(-c2ccc3ncc4ccc(=O)n(-c5cccc(C(F)(F)F)c5)c4c3c2)c1]

¹H NMR (400 MHz, DMSO-d₆) δ 9.18 (s, 1H), 8.34 (d, J=9.5 Hz, 1H), 8.16-8.12 (m, 2H), 8.09 (d, J=2.0 Hz, 2H), 7.90 (dd, J=17.4, 8.1 Hz, 3H), 7.81 (d, J=8.0 Hz, 1H), 7.23 (d, J=8.0 Hz, 2H), 7.13 (d, J=1.9 Hz, 1H), 6.95 (d, J=9.5 Hz, 1H), 2.59 (s, 3H); LC/MS (Method B): (electrospray+ve), m/z 458.7 (MH)⁺, t_{R}=5.044, UV₂₅₄=100%

### 9-(2,3-Dihydrobenzofuran-6-yl)-1-(3-(trifluoromethyl)phenyl)benzo[h][1,6]naphthyridin-2(1H)-one

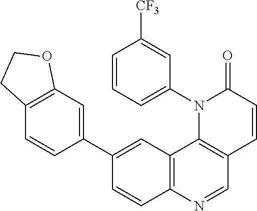 [SMILES: O=c1ccc2cnc3ccc(-c4ccc5c(c4)OCC5)cc3c2n1-c1cccc(C(F)(F)F)c1]

¹H NMR (400 MHz, DMSO-d₆) δ 9.11 (s, 1H), 8.32 (d, J=9.3 Hz, 1H), 8.14 (s, 1H), 8.06 (dt, J=8.2, 2.9 Hz, 3H), 7.94 (d, J=8.9 Hz, 2H), 7.87 (t, J=8.1 Hz, 1H), 7.78 (d, J=8.0 Hz, 1H), 7.72 (d, J=2.2 Hz, 1H), 6.99 (s, 1H), 6.91 (td, J=6.0, 2.6 Hz, 3H), 6.83 (s, 1H), 6.74-6.67 (m, 2H); LC/MS (Method B): (electrospray+ve), m/z 458.8 (MH)⁺, t_{R}=5.155, UV₂₅₄=100%

### 9-(Benzo[d][1,3]dioxol-5-yl)-1-(3-(trifluoromethyl)phenyl)benzo[h][1,6]naphthyridin-2(1H)-one

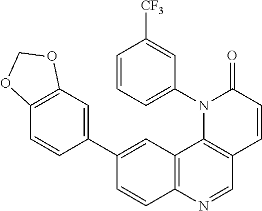 [SMILES: O=c1ccc2cnc3ccc(-c4ccc5c(c4)OCO5)cc3c2n1-c1cccc(C(F)(F)F)c1]

LC/MS (Method B): (electrospray+ve), m/z 461.1 (MH)⁺, t_{R}=5.147, UV₂₅₄=100%

### 9-(4-Fluorophenyl)-1-(3-(trifluoromethyl)phenyl)benzo[h][1,6]naphthyridin-2(1H)-one

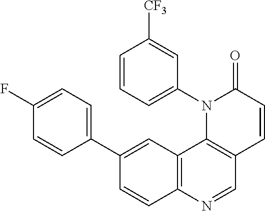 [SMILES: O=c1ccc2cnc3ccc(-c4ccc(F)cc4)cc3c2n1-c1cccc(C(F)(F)F)c1]

¹H NMR (400 MHz, DMSO-d₆) δ 9.15 (s, 1H), 8.33 (d, J=9.4 Hz, 1H), 8.13 (s, 1H), 8.10 (d, J=8.2 Hz, 1H), 8.06 (d, J=8.6 Hz, 1H), 7.98 (d, J=8.1 Hz, 1H), 7.90-7.84 (m, 1H), 7.79 (d, J=8.1 Hz, 1H), 7.21-7.15 (m, 2H), 7.11 (dd, J=8.6, 5.5 Hz, 2H), 6.98 (s, 1H), 6.94 (d, J=9.4 Hz, 1H); LC/MS (Method B): (electrospray+ve), m/z 435.1 (MH)⁺, t_{R}=5.400, UV₂₅₄=100%

### 9-(4-Hydroxyphenyl)-1-(3-(trifluoromethyl)phenyl)benzo[h][1,6]naphthyridin-2(1H)-one

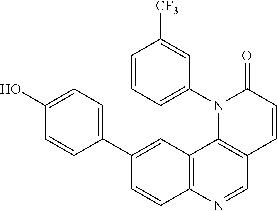 [SMILES: O=c1ccc2cnc3ccc(-c4ccc(O)cc4)cc3c2n1-c1cccc(C(F)(F)F)c1]

¹H NMR (400 MHz, DMSO-d₆) δ 9.63 (s, 1H), 9.11 (s, 1H), 8.31 (d, J=9.6 Hz, 1H), 8.14 (s, 1H), 8.05 (d, J=8.5 Hz, 2H), 7.93 (d, J=8.8 Hz, 1H), 7.86 (t, J=7.9 Hz, 1H), 7.75 (d, J=8.1 Hz, 1H), 6.99 (s, 1H), 6.93 (d, J=3.7 Hz, 2H), 6.90 (s, 1H), 6.71 (d, J=8.0 Hz, 2H); LC/MS (Method B): (electrospray+ve), m/z 433.1 (MH)⁺, t_{R}=4.410, UV₂₅₄=100%

### 9-(4-Morpholinophenyl)-1-(3-(trifluoromethyl)phenyl)benzo[h][1,6]naphthyridin-2(1H)-one

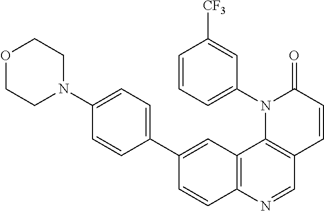 [SMILES: O=c1ccc2cnc3ccc(-c4ccc(N5CCOCC5)cc4)cc3c2n1-c1cccc(C(F)(F)F)c1]

LC/MS (Method B): (electrospray+ve), m/z 502.1 (MH)⁺, t_{R}=4.921, UV₂₅₄=100%

### 9-(1-Methyl-1H-indol-5-yl)-1-(3-(trifluoromethyl)phenyl)benzo[h][1,6]naphthyridin-2(1H)-one

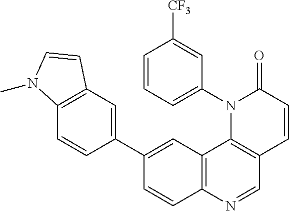 [SMILES: Cn1ccc2cc(-c3ccc4ncc5ccc(=O)n(-c6cccc(C(F)(F)F)c6)c5c4c3)ccc21]

¹H NMR (400 MHz, DMSO-d₆) δ 9.15 (s, 1H), 8.34 (d, J=9.4 Hz, 1H), 8.17 (s, 1H), 8.11 (d, J=5.8 Hz, 1H), 8.10 (s, 1H), 8.07 (d, J=9.0 Hz, 1H), 7.89 (t, J=7.9 Hz, 1H), 7.81 (d, J=8.0 Hz, 1H), 7.40 (d, J=8.6 Hz, 1H), 7.38 (d, J=3.2 Hz, 1H), 7.18 (d, J=10.1 Hz, 2H), 6.99 (s, 1H), 6.95 (d, J=9.4 Hz, 1H), 6.45 (d, J=3.1 Hz, 1H), 3.81 (s, 3H); LC/MS (Method B): (electrospray+ve), m/z 470.1 (MH)⁺, t_{R}=5.239, UV₂₅₄=100%

### 9-(Benzo[b]thiophen-5-yl)-1-(3-(trifluoromethyl)phenyl)benzo[h][1,6]naphthyridin-2(1H)-one

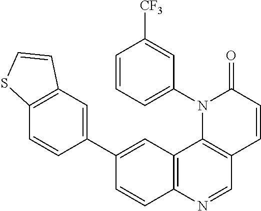 [SMILES: O=c1ccc2cnc3ccc(-c4ccc5sccc5c4)cc3c2n1-c1cccc(C(F)(F)F)c1]

¹H NMR (400 MHz, DMSO-d₆) δ 9.20 (s, 1H), 8.35 (d, J=9.6 Hz, 1H), 8.15 (d, J=8.5 Hz, 3H), 8.10 (d, J=9.6 Hz, 2H), 7.98 (d, J=8.4 Hz, 1H), 7.91 (t, J=7.9 Hz, 1H), 7.83 (t, J=7.1 Hz, 2H), 7.49 (s, 1H), 7.45 (d, J=5.4 Hz, 1H), 7.17 (s, 1H), 7.14 (d, J=8.5 Hz, 1H), 6.95 (d, J=9.2 Hz, 1H); LC/MS (Method B): (electrospray+ve), m/z 472.7 (MH)⁺, t_{R}=5.647, UV₂₅₄=100%

### 9-(4-(Methylsulfonyl)phenyl)-1-(3-(trifluoromethyl)phenyl)benzo[h][1,6]naphthyridin-2(1H)-one

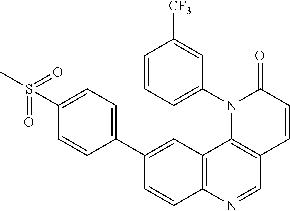 [SMILES: CS(=O)(=O)c1ccc(-c2ccc3ncc4ccc(=O)n(-c5cccc(C(F)(F)F)c5)c4c3c2)cc1]

¹H NMR (400 MHz, DMSO-d₆) δ 9.26 (s, 1H), 8.41 (d, J=9.5 Hz, 1H), 8.22 (d, J=8.7 Hz, 1H), 8.20-8.15 (m, 2H), 8.14 (d, J=9.3 Hz, 1H), 7.95 (d, J=6.4 Hz, 3H), 7.89 (d, J=7.9 Hz, 1H), 7.38 (d, J=8.0 Hz, 2H), 7.17 (s, 1H), 7.02 (d, J=9.2 Hz, 1H), 3.29 (s, 3H); LC/MS (Method B): (electrospray+ve), m/z 494.6 (MH)⁺, t_{R}=4.636, UV₂₅₄=100%

### 9-(Benzo[c][1,2,5]oxadiazol-5-yl)-1-(3-(trifluoromethyl)phenyl)benzo[h][1,6]naphthyridin-2(1H)-one

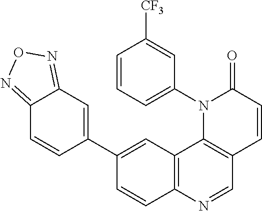 [SMILES: O=c1ccc2cnc3ccc(-c4ccc5nonc5c4)cc3c2n1-c1cccc(C(F)(F)F)c1]

LC/MS (Method B): (electrospray+ve), m/z 458.7 (MH)⁺, t_{R}=5.404, UV₂₅₄=100%

### 9-(6-(Piperidin-1-yl)pyridin-3-yl)-1-(3-(trifluoromethyl)phenyl)benzo[h][1,6]naphthyridin-2(1H)-one

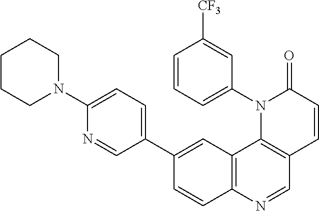 [SMILES: O=c1ccc2cnc3ccc(-c4ccc(N5CCCCC5)nc4)cc3c2n1-c1cccc(C(F)(F)F)c1]

LC/MS (Method B): (electrospray+ve), m/z 501.1 (MH)⁺, t_{R}=4.339, UV₂₅₄=100%

### 9-(2-(Dimethylamino)pyrimidin-5-yl)-1-(3-(trifluoromethyl)phenyl)benzo[h][1,6]naphthyridin-2(1H)-one

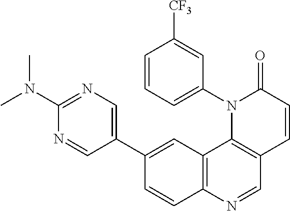 [SMILES: CN(C)c1ncc(-c2ccc3ncc4ccc(=O)n(-c5cccc(C(F)(F)F)c5)c4c3c2)cn1]

¹H NMR (400 MHz, DMSO-d₆) δ 9.14 (s, 1H), 8.32 (d, J=9.5 Hz, 1H), 8.08 (d, J=8.0 Hz, 4H), 8.00 (d, J=7.9 Hz, 1H), 7.96 (d, J=8.7 Hz, 1H), 7.89 (t, J=7.9 Hz, 1H), 7.80 (d, J=8.0 Hz, 1H), 6.93 (d, J=9.4 Hz, 1H), 6.89 (s, 1H), 3.14 (s, 6H); LC/MS (Method B): (electrospray+ve), m/z 462.1 (MH)⁺, t_{R}=4.774, UV₂₅₄=100%

### 9-(6-Methylpyridin-3-yl)-1-(3-(trifluoromethyl)phenyl)benzo[h][1,6]naphthyridin-2(1H)-one

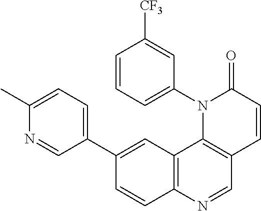 [SMILES: Cc1ccc(-c2ccc3ncc4ccc(=O)n(-c5cccc(C(F)(F)F)c5)c4c3c2)cn1]

¹H NMR (400 MHz, DMSO-d₆) δ 9.20 (s, 1H), 8.34 (d, J=9.4 Hz, 1H), 8.32 (s, 1H), 8.15 (d, J=8.7 Hz, 1H), 8.11 (s, 1H), 8.06 (d, J=8.8 Hz, 1H), 8.03 (d, J=7.5 Hz, 1H), 7.91-7.81 (m, 2H), 7.43 (d, J=8.3 Hz, 1H), 7.37 (d, J=8.3 Hz, 1H), 7.02 (s, 1H), 6.96 (d, J=9.4 Hz, 1H), 2.53 (s, 3H); LC/MS (Method B): (electrospray+ve), m/z 432.1 (MH)⁺, t_{R}=3.741, UV₂₅₄=100%

### 4-(2-Oxo-1-(3-(trifluoromethyl)phenyl)-1,2-dihydrobenzo[h][1,6]naphthyridin-9-yl)benzoic acid

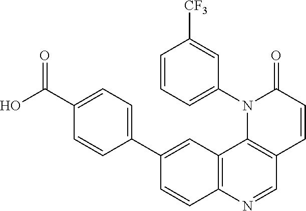 [SMILES: O=C(O)c1ccc(-c2ccc3ncc4ccc(=O)n(-c5cccc(C(F)(F)F)c5)c4c3c2)cc1]

¹H NMR (400 MHz, DMSO-d₆) δ 13.06 (s, 1H), 9.18 (s, 1H), 8.34 (d, J=9.3 Hz, 1H), 8.16 (s, 1H), 8.14 (d, J=9.0 Hz, 1H), 8.10 (d, J=7.8 Hz, 1H), 8.06 (dd, J=8.8, 1.5 Hz, 1H), 7.90 (d, J=8.0 Hz, 2H), 7.87 (d, J=7.9 Hz, 1H), 7.80 (d, J=8.0 Hz, 1H), 7.20 (d, J=8.0 Hz, 2H), 7.12 (s, 1H), 6.95 (d, J=9.4 Hz, 1H); LC/MS (Method B): (electrospray+ve), m/z 461.1 (MH)⁺, t_{R}=4.491, UV₂₅₄=100%

### 4-(2-Oxo-1-(3-(trifluoromethyl)phenyl)-1,2-dihydrobenzo[h][1,6]naphthyridin-9-yl)benzenesulfonamide

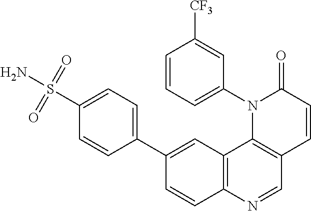 [SMILES: NS(=O)(=O)c1ccc(-c2ccc3ncc4ccc(=O)n(-c5cccc(C(F)(F)F)c5)c4c3c2)cc1]

¹H NMR (400 MHz, DMSO-d₆) δ 9.18 (s, 1H), 8.34 (d, J=9.3 Hz, 1H), 8.15 (d, J=9.0 Hz, 2H), 8.07 (dd, J=8.8, 1.8 Hz, 2H), 7.89 (t, J=7.9 Hz, 1H), 7.81 (d, J=7.1 Hz, 1H), 7.78 (d, J=8.4 Hz, 2H), 7.42 (s, 2H), 7.26 (d, J=8.1 Hz, 2H), 7.13 (d, J=1.9 Hz, 1H), 6.95 (d, J=9.5 Hz, 1H); LC/MS (Method B): (electrospray+ve), m/z 496.0 (MH)⁺, t_{R}=3.985, UV₂₅₄=100%

### N,N-Dimethyl-4-(2-oxo-1-(3-(trifluoromethyl)phenyl)-1,2-dihydrobenzo[h][1,6]naphthyridin-9-yl)benzamide

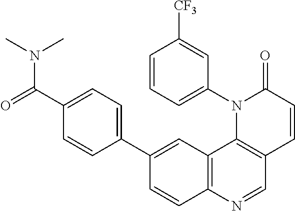 [SMILES: CN(C)C(=O)c1ccc(-c2ccc3ncc4ccc(=O)n(-c5cccc(C(F)(F)F)c5)c4c3c2)cc1]

¹H NMR (400 MHz, DMSO-d₆) δ 9.18 (s, 1H), 8.34 (d, J=9.4 Hz, 1H), 8.16-8.11 (m, 2H), 8.09-8.03 (m, 2H), 7.89 (s, 1H), 7.81 (s, 1H), 7.37 (d, J=7.9 Hz, 2H), 7.13 (d, J=7.8 Hz, 2H), 7.08 (s, 1H), 6.95 (d, J=9.3 Hz, 1H), 2.95 (d, J=31.7 Hz, 6H); LC/MS (Method B): (electrospray+ve), m/z 488.1 (MH)⁺, t_{R}=4.543, UV₂₅₄=100%

### 9-([1,1′-Biphenyl]-4-yl)-1-(3-(trifluoromethyl)phenyl)benzo[h][1,6]naphthyridin-2(1H)-one

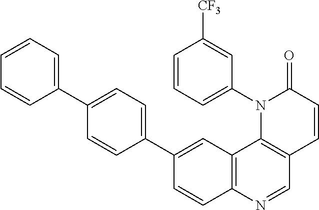 [SMILES: O=c1ccc2cnc3ccc(-c4ccc(-c5ccccc5)cc4)cc3c2n1-c1cccc(C(F)(F)F)c1]

¹H NMR (400 MHz, DMSO-d₆) δ 9.16 (s, 1H), 8.34 (d, J=9.4 Hz, 1H), 8.16 (s, 1H), 8.14-8.10 (m, 2H), 8.07 (d, J=8.7 Hz, 1H), 7.90 (t, J=7.9 Hz, 1H), 7.81 (d, J=7.8 Hz, 1H), 7.69 (d, J=7.6 Hz, 2H), 7.65 (d, J=8.0 Hz, 2H), 7.49 (t, J=7.6 Hz, 2H), 7.39 (t, J=7.4 Hz, 1H), 7.19 (d, J=8.0 Hz, 2H), 7.13 (s, 1H), 6.94 (d, J=9.4 Hz, 1H); LC/MS (Method B): (electrospray+ve), m/z 493.1 (MH)⁺, t_{R}=6.137, UV₂₅₄=100%

### 9-(p-Tolyl)-1-(3-(trifluoromethyl)phenyl)benzo[h][1,6]naphthyridin-2(1H)-one

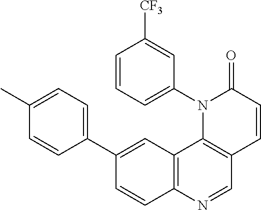 [SMILES: Cc1ccc(-c2ccc3ncc4ccc(=O)n(-c5cccc(C(F)(F)F)c5)c4c3c2)cc1]

¹H NMR (400 MHz, DMSO-d₆) δ 9.14 (s, 1H), 8.32 (d, J=9.5 Hz, 1H), 8.14 (s, 1H), 8.09 (d, J=8.7 Hz, 1H), 8.05 (d, J=7.9 Hz, 1H), 7.99 (d, J=8.7 Hz, 1H), 7.86 (t, J=7.9 Hz, 1H), 7.78 (d, J=8.0 Hz, 1H), 7.15 (d, J=7.8 Hz, 2H), 7.07 (s, 1H), 6.99 (d, J=7.8 Hz, 2H), 6.93 (d, J=9.4 Hz, 1H), 2.31 (s, 3H); LC/MS (Method B): (electrospray+ve), m/z 431.1 (MH)⁺, t_{R}=5.546, UV₂₅₄=100%

### 9-(5-Chloropyridin-3-yl)-1-(3-(trifluoromethyl)phenyl)benzo[h][1,6]naphthyridin-2(1H)-one

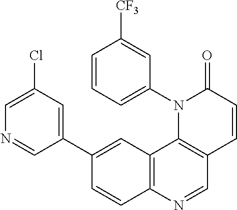 [SMILES: O=c1ccc2cnc3ccc(-c4cncc(Cl)c4)cc3c2n1-c1cccc(C(F)(F)F)c1]

LC/MS (Method B): (electrospray+ve), m/z 451.7 (MH)⁺, t_{R}=5.071, UV₂₅₄=100%

### 9-(3-(Pyrrolidin-1-yl)phenyl)-1-(3-(trifluoromethyl)phenyl)benzo[h][1,6]naphthyridin-2(1H)-one

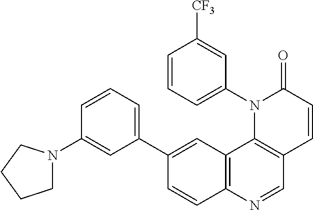 [SMILES: O=c1ccc2cnc3ccc(-c4cccc(N5CCCC5)c4)cc3c2n1-c1cccc(C(F)(F)F)c1]

LC/MS (Method B): (electrospray+ve), m/z 485.8 (MH)⁺, t_{R}=5.719, UV₂₅₄=100%

### 9-(4-(Piperazine-1-carbonyl)phenyl)-1-(3-(trifluoromethyl)phenyl)benzo[h][1,6]naphthyridin-2(1H)-one

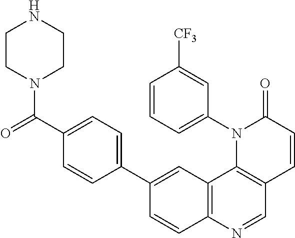 [SMILES: O=C(c1ccc(-c2ccc3ncc4ccc(=O)n(-c5cccc(C(F)(F)F)c5)c4c3c2)cc1)N1CCNCC1]

LC/MS (Method B): (electrospray+ve), m/z 528.7 (M), t_{R}=3.708, UV₂₅₄=100%

### 9-(4-(Morpholine-4-carbonyl)phenyl)-1-(3-(trifluoromethyl)phenyl)benzo[h][1,6]naphthyridin-2(1H)-one

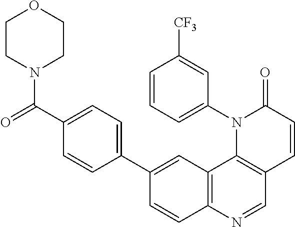 [SMILES: O=C(c1ccc(-c2ccc3ncc4ccc(=O)n(-c5cccc(C(F)(F)F)c5)c4c3c2)cc1)N1CCOCC1]

LC/MS (Method B): (electrospray+ve), m/z 529.7 (MH)⁺, t_{R}=4.496, UV₂₅₄=100%

### 9-(6-Hydroxypyridin-3-yl)-1-(3-(trifluoromethyl)phenyl)benzo[h][1,6]naphthyridin-2(1H)-one

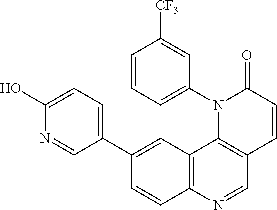 [SMILES: O=c1ccc2cnc3ccc(-c4ccc(O)nc4)cc3c2n1-c1cccc(C(F)(F)F)c1]

¹H NMR (400 MHz, DMSO-d₆) δ 11.94 (s, 1H), 9.12 (s, 1H), 8.32 (d, J=9.4 Hz, 1H), 8.09 (s, 2H), 8.03 (s, 1H), 7.91 (d, J=8.2 Hz, 2H), 7.82 (d, J=8.0 Hz, 1H), 7.26 (s, 1H), 7.02 (d, J=10.0 Hz, 1H), 6.92 (s, 1H), 6.80 (s, 1H), 6.31 (d, J=9.5 Hz, 1H); LC/MS (Method B): (electrospray+ve), m/z 433.7 (MH)⁺, t_{R}=3.808, UV₂₅₄=100%

### 3-(2-Oxo-1-(3-(trifluoromethyl)phenyl)-1,2-dihydrobenzo[h][1,6]naphthyridin-9-yl)benzenesulfonamide

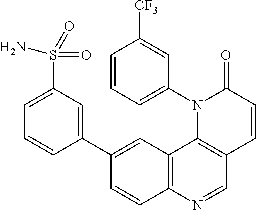 [SMILES: NS(=O)(=O)c1cccc(-c2ccc3ncc4ccc(=O)n(-c5cccc(C(F)(F)F)c5)c4c3c2)c1]

¹H NMR (400 MHz, DMSO-d₆) δ 9.18 (s, 1H), 8.33 (d, J=9.5 Hz, 1H), 8.17 (d, J=8.6 Hz, 1H), 8.07 (s, 1H), 8.02 (d, J=7.6 Hz, 1H), 7.98 (dd, J=8.6, 2.0 Hz, 1H), 7.88 (t, J=7.7 Hz, 1H), 7.83 (d, J=8.3 Hz, 1H), 7.79 (d, J=7.4 Hz, 2H), 7.51 (t, J=8.0 Hz, 1H), 7.36 (s, 2H), 7.02-6.99 (m, 2H), 6.94 (d, J=9.4 Hz, 1H); LC/MS (Method B): (electrospray+ve), m/z 496.1 (MH)⁺, t_{R}=4.315, UV₂₅₄=100%

### 9-(1-Oxo-1,2,3,4-tetrahydroisoquinolin-6-yl)-1-(3-(trifluoromethyl)phenyl)benzo[h][1,6]naphthyridin-2(1H)-one

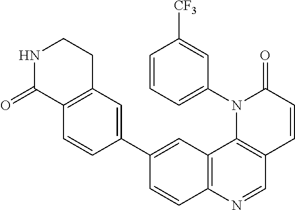 [SMILES: O=C1NCCc2cc(-c3ccc4ncc5ccc(=O)n(-c6cccc(C(F)(F)F)c6)c5c4c3)ccc21]

¹H NMR (400 MHz, DMSO-d₆) δ 9.17 (s, 1H), 8.33 (d, J=9.4 Hz, 1H), 8.14-8.11 (m, 2H), 8.09 (d, J=7.8 Hz, 1H), 8.04 (dd, J=8.7, 1.9 Hz, 1H), 7.95 (d, J=2.8 Hz, 1H), 7.90 (t, J=7.9 Hz, 1H), 7.82 (d, J=1.9 Hz, 1H), 7.79 (d, J=8.1 Hz, 1H), 7.11 (d, J=1.9 Hz, 1H), 7.03 (dd, J=8.0, 1.9 Hz, 1H), 6.97 (d, J=1.8 Hz, 1H), 6.94 (d, J=9.5 Hz, 1H), 3.39 (td, J=6.7, 2.8 Hz, 4H); LC/MS (Method B): (electrospray+ve), m/z 486.1 (MH)⁺, t_{R}=4.359, UV₂₅₄=100%

### 9-(2-Oxo-1,2,3,4-tetrahydroquinolin-6-yl)-1-(3-(trifluoromethyl)phenyl)benzo[h][1,6]naphthyridin-2(1H)-one

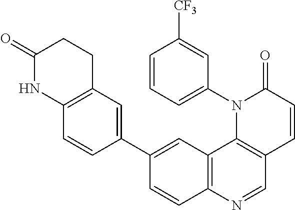 [SMILES: O=C1CCc2cc(-c3ccc4ncc5ccc(=O)n(-c6cccc(C(F)(F)F)c6)c5c4c3)ccc2N1]

¹H NMR (400 MHz, DMSO-d₆) δ 10.13 (s, 1H), 9.13 (s, 1H), 8.31 (d, J=9.5 Hz, 1H), 8.11 (d, J=2.1 Hz, 1H), 8.07 (d, J=6.0 Hz, 1H), 8.05 (d, J=5.7 Hz, 1H), 7.95 (dd, J=8.7, 1.9 Hz, 1H), 7.89 (t, J=7.9 Hz, 1H), 7.78 (dd, J=7.7, 1.8 Hz, 1H), 7.03 (d, J=1.9 Hz, 1H), 6.93-6.91 (m, 1H), 6.90 (d, J=6.1 Hz, 1H), 6.82-6.80 (m, 2H), 3.43 (s, 4H); LC/MS (Method B): (electrospray+ve), m/z 486.1 (MH)⁺, t_{R}=4.287, UV₂₅₄=100%

### 9-(4-(Aminomethyl)phenyl)-1-(3-(trifluoromethyl)phenyl)benzo[h][1,6]naphthyridin-2(1H)-one

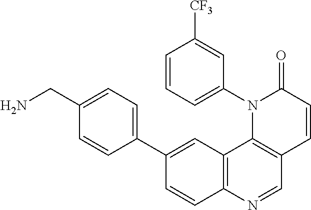 [SMILES: NCc1ccc(-c2ccc3ncc4ccc(=O)n(-c5cccc(C(F)(F)F)c5)c4c3c2)cc1]

¹H NMR (400 MHz, DMSO-d₆) δ 9.16 (s, 1H), 8.33 (d, J=9.4 Hz, 1H), 8.17 (d, J=1.9 Hz, 1H), 8.12 (d, J=8.7 Hz, 3H), 8.03 (dd, J=8.7, 1.9 Hz, 1H), 7.98 (d, J=7.8 Hz, 1H), 7.85 (t, J=7.8 Hz, 1H), 7.80 (d, J=8.1 Hz, 1H), 7.42 (d, J=8.2 Hz, 2H), 7.16-7.13 (m, 2H), 7.11 (d, J=1.9 Hz, 1H), 6.94 (d, J=9.4 Hz, 1H), 4.05 (d, J=5.8 Hz, 2H); LC/MS (Method B): (electrospray+ve), m/z 446.1 (MH)⁺, t_{R}=3.630, UV₂₅₄=100%

### 9-(4-((Dimethylamino)methyl)phenyl)-1-(3-(trifluoromethyl)phenyl)benzo[h][1,6]naphthyridin-2(1H)-one

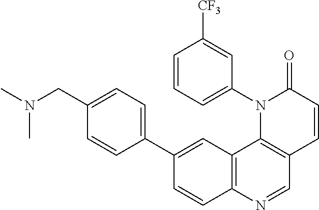 [SMILES: CN(C)Cc1ccc(-c2ccc3ncc4ccc(=O)n(-c5cccc(C(F)(F)F)c5)c4c3c2)cc1]

¹H NMR (400 MHz, DMSO-d₆) δ 9.17 (s, 1H), 8.33 (d, J=9.5 Hz, 1H), 8.17 (s, 1H), 8.13 (d, J=8.6 Hz, 1H), 8.03 (dd, J=8.7, 1.9 Hz, 1H), 7.98 (d, J=7.8 Hz, 1H), 7.86 (t, J=7.9 Hz, 1H), 7.79 (d, J=7.9 Hz, 1H), 7.46 (d, J=8.2 Hz, 2H), 7.17 (d, J=8.2 Hz, 2H), 7.08 (d, J=1.9 Hz, 1H), 6.94 (d, J=9.4 Hz, 1H), 4.29 (d, J=5.2 Hz, 2H), 2.72 (d, J=4.6 Hz, 6H); LC/MS (Method B): (electrospray+ve), m/z 474.1 (MH)⁺, t_{R}=3.772, UV₂₅₄=100%

### N-Methyl-4-(2-oxo-1-(3-(trifluoromethyl)phenyl)-1,2-dihydrobenzo[h][1,6]naphthyridin-9-yl)benzamide

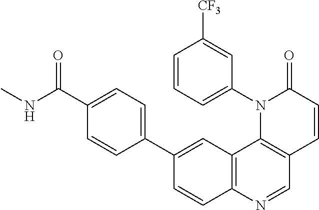 [SMILES: CNC(=O)c1ccc(-c2ccc3ncc4ccc(=O)n(-c5cccc(C(F)(F)F)c5)c4c3c2)cc1]

¹H NMR (400 MHz, DMSO-d₆) δ 9.18 (s, 1H), 8.47 (d, J=4.7 Hz, 1H), 8.33 (d, J=9.4 Hz, 1H), 8.14 (s, 1H), 8.12 (d, J=8.8 Hz, 1H), 8.07-8.03 (m, 2H), 7.86 (d, J=7.8 Hz, 1H), 7.82-7.78 (m, 3H), 7.15 (d, J=8.3 Hz, 2H), 7.10 (d, J=1.9 Hz, 1H), 6.94 (d, J=9.4 Hz, 1H), 2.78 (d, J=4.4 Hz, 3H); LC/MS (Method B): (electrospray+ve), m/z 474.1 (MH)⁺, t_{R}=4.339, UV₂₅₄=100%

### 9-(2-Oxoindolin-5-yl)-1-(3-(trifluoromethyl)phenyl)benzo[h][1,6]naphthyridin-2(1H)-one

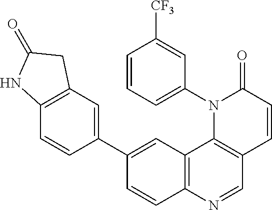 [SMILES: O=C1Cc2cc(-c3ccc4ncc5ccc(=O)n(-c6cccc(C(F)(F)F)c6)c5c4c3)ccc2N1]

¹H NMR (400 MHz, DMSO-d₆) δ 10.47 (s, 1H), 9.14 (s, 1H), 8.31 (d, J=9.5 Hz, 1H), 8.11 (d, J=8.0 Hz, 2H), 8.07 (d, J=8.7 Hz, 1H), 7.95 (dd, J=8.7, 2.0 Hz, 1H), 7.86 (d, J=7.9 Hz, 1H), 7.78 (d, J=7.9 Hz, 1H), 6.99 (d, J=1.9 Hz, 1H), 6.97-6.94 (m, 1H), 6.93 (d, J=9.5 Hz, 1H), 6.84 (s, 1H), 6.76 (d, J=8.1 Hz, 1H), 3.48 (q, J=22.3 Hz, 3H); LC/MS (Method B): (electrospray+ve), m/z 472.0 (MH)⁺, t_{R}=4.214, UV₂₅₄=100%

### 9-(1-Oxoisoindolin-5-yl)-1-(3-(trifluoromethyl)phenyl)benzo[h][1,6]naphthyridin-2(1H)-one

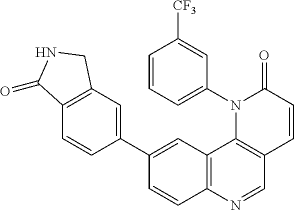 [SMILES: O=C1NCc2cc(-c3ccc4ncc5ccc(=O)n(-c6cccc(C(F)(F)F)c6)c5c4c3)ccc21]

¹H NMR (400 MHz, DMSO-d₆) δ 9.18 (s, 1H), 8.61 (s, 1H), 8.33 (d, J=9.4 Hz, 1H), 8.15-8.12 (m, 2H), 8.11 (d, J=7.4 Hz, 1H), 8.05 (dd, J=8.7, 2.0 Hz, 1H), 7.87 (t, J=7.8 Hz, 1H), 7.80 (d, J=8.0 Hz, 1H), 7.62 (d, J=8.3 Hz, 1H), 7.20 (s, 1H), 7.19 (d, J=3.8 Hz, 1H), 7.09 (d, J=1.9 Hz, 1H), 6.94 (d, J=9.5 Hz, 1H), 4.44-4.31 (m, 2H); LC/MS (Method B): (electrospray+ve), m/z 472.1 (MH)⁺, t_{R}=4.204, UV₂₅₄=100%

### 2,4-Difluoro-N-(2-methoxy-5-(2-oxo-1-(3-(trifluoromethyl)phenyl)-1,2-dihydrobenzo[h][1,6]naphthyridin-9-yl)pyridin-3-yl)benzenesulfonamide

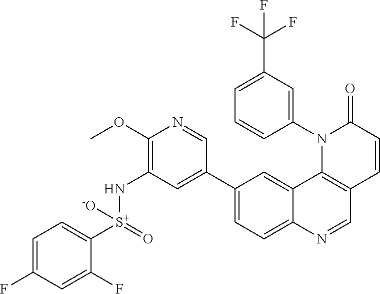

LC/MS (Method B): (electrospray+ve), m/z 638.6 (MH)⁺, t_{R}=5.26 min, UV₂₅₄=95%.

### 1-(1-Acetylpiperidin-4-yl)-9-(6-aminopyridin-3-yl)benzo[h][1,6]naphthyridin-2(1H)-one

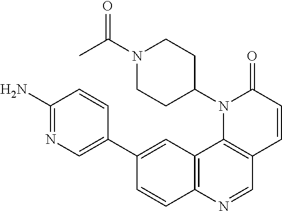 [SMILES: CC(=O)N1CCC(n2c(=O)ccc3cnc4ccc(-c5ccc(N)nc5)cc4c32)CC1]

LC/MS (Method B): (electrospray+ve), m/z 414.2 (MH)⁺, t_{R}=2.90 min, UV₂₅₄ 98%.

### 9-(6-Aminopyridin-3-yl)-3-methyl-1-(3-(trifluoromethyl)phenyl)benzo[h][1,6]naphthyridin-2(1H)-one

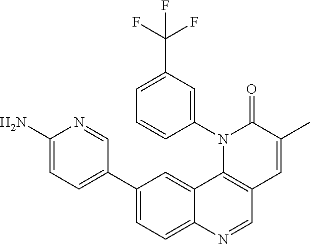 [SMILES: Cc1cc2cnc3ccc(-c4ccc(N)nc4)cc3c2n(-c2cccc(C(F)(F)F)c2)c1=O]

LC/MS (Method B): (electrospray+ve), m/z 447.1 (MH)⁺, t_{R}=3.77 min, UV₂₅₄=85%.

### 9-(6-Aminopyridin-3-yl)-1-cyclopropylbenzo[h][1,6]naphthyridin-2(1H)-one

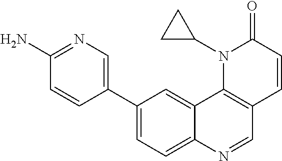 [SMILES: Nc1ccc(-c2ccc3ncc4ccc(=O)n(C5CC5)c4c3c2)cn1]

LC/MS (Method B): (electrospray+ve), m/z 329.1 (MH)⁺, t_{R}=2.79 min, UV₂₅₄=95%.

### 9-(6-Aminopyridin-3-yl)-1-(tetrahydro-2H-pyran-4-yl)benzo[h][1,6]naphthyridin-2(1H)-one

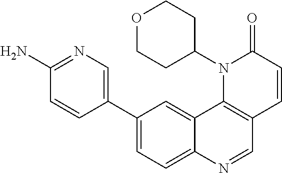 [SMILES: Nc1ccc(-c2ccc3ncc4ccc(=O)n(C5CCOCC5)c4c3c2)cn1]

LC/MS (Method B): (electrospray+ve), m/z 373.1 (MH)⁺, t_{R}=2.90 min, UV₂₅₄=90%.

### 9-(6-Aminopyridin-3-yl)-1-cyclohexylbenzo[h][1,6]naphthyridin-2(1H)-one

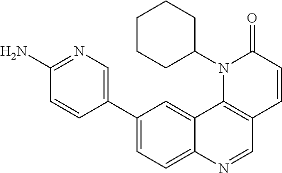 [SMILES: Nc1ccc(-c2ccc3ncc4ccc(=O)n(C5CCCCC5)c4c3c2)cn1]

LC/MS (Method B): (electrospray+ve), m/z 371.2 (MH)⁺, t_{R}=3.45 min, UV₂₅₄=80%.

### 9-(6-Aminopyridin-3-yl)-1-cyclopentylbenzo[h][1,6]naphthyridin-2(1H)-one

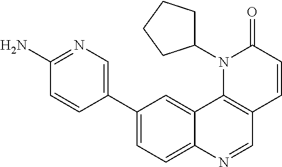 [SMILES: Nc1ccc(-c2ccc3ncc4ccc(=O)n(C5CCCC5)c4c3c2)cn1]

LC/MS (Method B): (electrospray+ve), m/z 357.2 (MH)⁺, t_{R}=3.26 min, UV₂₅₄=95%.

### 9-(6-Aminopyridin-3-yl)-3-ethyl-1-(3-(trifluoromethyl)phenyl)benzo[h][1,6]naphthyridin-2(1H)-one

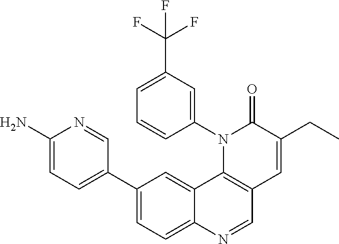 [SMILES: CCc1cc2cnc3ccc(-c4ccc(N)nc4)cc3c2n(-c2cccc(C(F)(F)F)c2)c1=O]

LC/MS (Method B): (electrospray+ve), m/z 460.8 (MH)⁺, t_{R}=4.08 min, UV₂₅₄=90%.

General Procedure for the Synthesis of Compound 7:

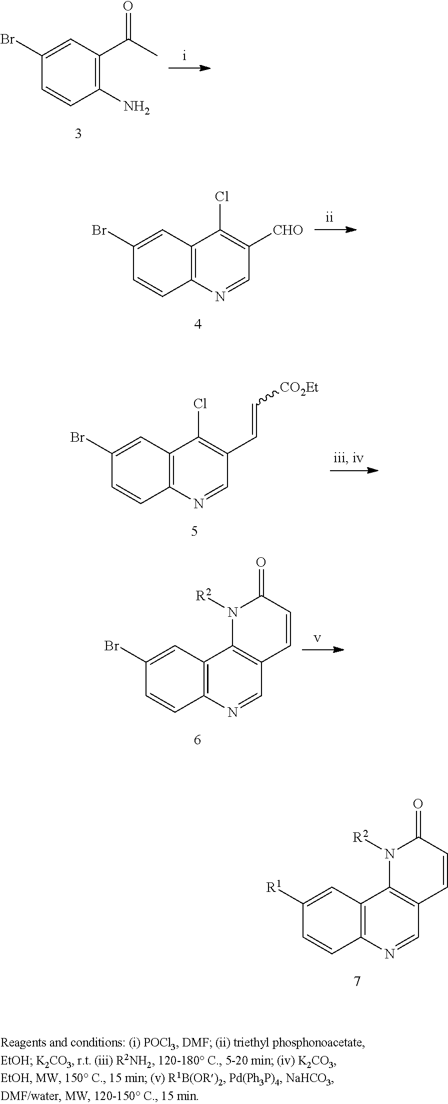

A two-necked flask equipped with a stirrer, condenser, and rubber septum was charged with dry DMF (300 mL) at 0° C. under a nitrogen atmosphere. Phosphoryl chloride (100 mL, 1002.4 mmol) was added dropwise to the flask at 0° C. The mixture was allowed to warm up to room temperature and stirred at this temperature for 30 min. A solution of 3 (25.1 g, 117.3 mmol) in DMF (100 mL) was added dropwise and the mixture was heated at 60° C. for 4 h under nitrogen. The cooled mixture was added to crush ice and then neutralized with saturated NaHCO₃ solution. The solid was collected by filtration. The crude product was dissolved in dichloromethane and the solution was washed with water, dried over MgSO₄, filtered, and concentrated to give 4 (15.2 g, 48%) as a solid. ¹H NMR (400 MHz, Chloroform-d) δ 10.69 (s, 1H), 9.26 (s, 1H), 8.54 (d, J=2.1 Hz, 1H), 8.04 (d, J=12.4 Hz, 1H), 7.98 (dd, J=11.8, 2.1 Hz, 1H); LC/MS (Method B): (electrospray+ve), m/z 269.9 (MH)⁺, t_{R}=5.60 min, UV₂₅₄=98%.

To a solution of 4 (5.04 g, 18.7 mmol) in 300 mL of ethanol were added triethyl phosphonoacetate (6.28 g, 28.0 mmol) and K₂CO₃ (12.90 g, 93.0 mmol). The mixture was stirred at room temperature for overnight. The solid was filtered, washed with water and ethanol, and dried under vacuum to give 5 (4.41 g, 69%) as a solid. LC/MS (Method B): (electrospray+ve), m/z 340.0 (MH)⁺, t_{R}=6.52 min, UV₂₅₄=100%.

A mixture of 5 (0.3 mmol) and amines (0.6 mmol) was heated at 120-180° C. for 5-20 min. After cooled down to room temperature, ethanol (5 mL) and K₂CO₃ (1.8 mmol) were added. The mixture was heated at 80° C. for overnight. Ethyl acetate (50 mL) was added to the reaction and the mixture was washed with water (2×30 mL). The organic layer was dried over MgSO₄, filtered, and concentrated. The crude product was purified by column chromatography on silica gel using dichloromethane in methanol (0-20%) as eluent to give 6.

A mixture of 6 (0.2 mmol, 1.0 equiv), boronic acid or boronic acid pinacol ester (0.6 mmol, 3.0 equiv), tetrakis(triphenylphosphine)palladium (12 mg, 0.01 mmol, 0.05 equiv), DMF (2.5 mL) and saturated NaHCO₃ aqueous solution (0.5 mL) was charged in a microwave vial. Nitrogen was bubbled through the mixture for 3 min. The vial was capped and heated in a microwave at 120-150° C. for 15 min. The reaction mixture was filtered through a plug of Celite and the filtrate was purified by reverse phase column chromatography using acetonitrile (containing 0.1% TFA)/water (containing 0.1% TFA) as an eluent to give 7.

### 3-(9-(6-Aminopyridin-3-yl)-2-oxobenzo[h][1,6]naphthyridin-1(2H)-yl)-N,N-dimethylbenzamide

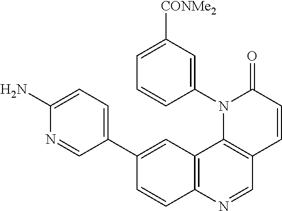

¹H NMR (400 MHz, DMSO-d₆) δ 9.18 (s, 1H), 8.34 (d, J=9.5 Hz, 1H), 8.16-8.04 (m, 1H), 8.03-7.89 (m, 2H), 7.78-7.53 (m, 5H), 7.26-6.91 (m, 4H), 2.97 (s, 3H), 2.83 (s, 3H); LC/MS (Method B): (electrospray+ve), m/z 436.2 (MH)⁺, t_{R}=3.04 min, UV₂₅₄=90%.

### 9-(6-Aminopyridin-3-yl)-1-(3-isopropylphenyl)benzo[h][1,6]naphthyridin-2(1H)-one

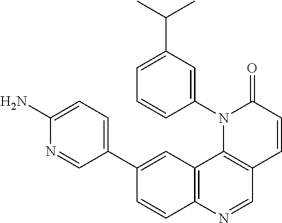 [SMILES: CC(C)c1cccc(-n2c(=O)ccc3cnc4ccc(-c5ccc(N)nc5)cc4c32)c1]

LC/MS (Method B): (electrospray+ve), m/z 407.1 (MH)⁺, t_{R}=3.58 min, UV₂₅₄=95%.

### 3-(9-(6-Aminopyridin-3-yl)-2-oxobenzo[h][1,6]naphthyridin-1(2H)-yl)benzenesulfonamide

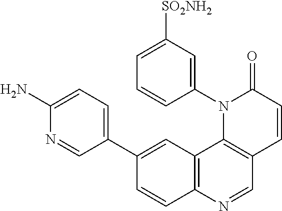 [SMILES: Nc1ccc(-c2ccc3ncc4ccc(=O)n(-c5cccc(S(N)(=O)=O)c5)c4c3c2)cn1]

¹H NMR (400 MHz, DMSO-d₆) δ 9.20 (s, 1H), 8.36 (d, J=9.5 Hz, 1H), 8.20-7.92 (m, 7H), 7.85 (t, J=7.9 Hz, 1H), 7.73 (ddd, J=7.9, 2.1, 1.1 Hz, 1H), 7.65 (s, 2H), 7.55-7.31 (m, 1H), 6.98 (dd, J=9.3, 3.6 Hz, 2H), 6.87 (d, J=1.9 Hz, 1H); LC/MS (Method B): (electrospray+ve), m/z 444.1 (MH)⁺, t_{R}=2.93 min, UV₂₅₄=90%.

### 9-(6-Aminopyridin-3-yl)-1-phenylbenzo[h][1,6]naphthyridin-2(1H)-one

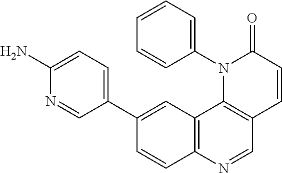 [SMILES: Nc1ccc(-c2ccc3ncc4ccc(=O)n(-c5ccccc5)c4c3c2)cn1]

LC/MS (Method B): (electrospray+ve), m/z 365.1 (MH)⁺, t_{R}=3.17 min, UV₂₅₄=98%.

### 9-(6-Aminopyridin-3-yl)-1-(piperidin-4-ylmethyl)benzo[h][1,6]naphthyridin-2(1H)-one

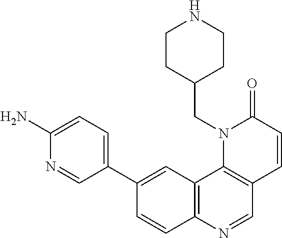 [SMILES: Nc1ccc(-c2ccc3ncc4ccc(=O)n(CC5CCNCC5)c4c3c2)cn1]

LC/MS (Method B): (electrospray+ve), m/z 386.1 (MH)⁺, t_{R}=2.64 min, UV₂₅₄=98%.

### 9-(6-Aminopyridin-3-yl)-1-ethylbenzo[h][1,6]naphthyridin-2(1H)-one

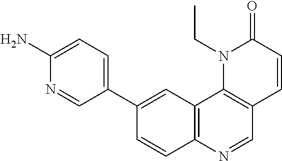 [SMILES: CCn1c(=O)ccc2cnc3ccc(-c4ccc(N)nc4)cc3c21]

LC/MS (Method B): (electrospray+ve), m/z 317.1 (MH)⁺, t_{R}=2.94 min, UV₂₅₄=98%.

### 9-(6-Aminopyridin-3-yl)-1-(2-methyl-5-(trifluoromethyl)phenyl)benzo[h][1,6]naphthyridin-2(1H)-one

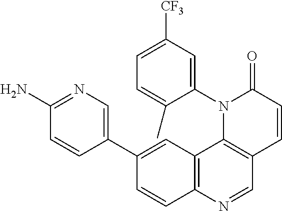 [SMILES: Cc1ccc(C(F)(F)F)cc1-n1c(=O)ccc2cnc3ccc(-c4ccc(N)nc4)cc3c21]

LC/MS (Method B): (electrospray+ve), m/z 447.1 (MH)⁺, t_{R}=3.76 min, UV₂₅₄=98%.

### 9-(6-Aminopyridin-3-yl)-1-(m-tolyl)benzo[h][1,6]naphthyridin-2(1H)-one

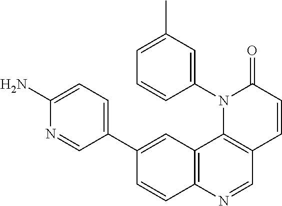 [SMILES: Cc1cccc(-n2c(=O)ccc3cnc4ccc(-c5ccc(N)nc5)cc4c32)c1]

LC/MS (Method A): (electrospray+ve), m/z 379.1 (MH)⁺, t_{R}=3.17 min, UV₂₅₄=98%.

### 9-(6-Aminopyridin-3-yl)-1-(3-chlorophenyl)benzo[h][1,6]naphthyridin-2(1H)-one

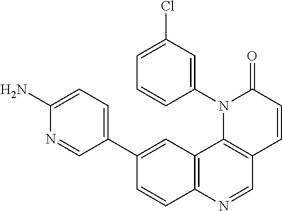 [SMILES: Nc1ccc(-c2ccc3ncc4ccc(=O)n(-c5cccc(Cl)c5)c4c3c2)cn1]

¹H NMR (400 MHz, DMSO-d₆) δ 9.19 (s, 1H), 8.34 (d, J=9.5 Hz, 1H), 8.14 (d, J=8.7 Hz, 1H), 8.07-8.13 (m, 2H), 8.00 (dd, J=8.7, 2.0 Hz, 1H), 7.89-7.65 (m, 4H), 7.55-7.47 (m, 2H), 7.04-6.98 (m, 2H), 6.96 (d, J=9.4 Hz, 1H); LC/MS (Method B): (electrospray+ve), m/z 399.0 (MH)⁺, t_{R}=3.40 min, UV₂₅₄=98%.

### 3-(9-(6-Aminopyridin-3-yl)-2-oxobenzo[h][1,6]naphthyridin-1(2H)-yl)benzonitrile

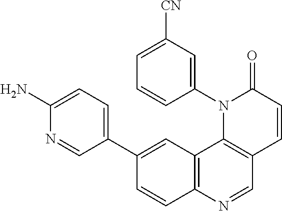 [SMILES: N#Cc1cccc(-n2c(=O)ccc3cnc4ccc(-c5ccc(N)nc5)cc4c32)c1]

¹H NMR (400 MHz, DMSO-d₆) δ 9.20 (s, 1H), 8.36 (d, J=9.5 Hz, 1H), 8.04-8.23 (m, 5H), 8.00 (dd, J=8.7, 2.0 Hz, 1H), 7.94-7.82 (m, 2H), 7.80 (dd, J=2.5, 0.8 Hz, 1H), 7.50 (dd, J=9.3, 2.3 Hz, 1H), 6.99 (dd, J=9.6, 8.5 Hz, 2H), 6.92 (d, J=1.9 Hz, 1H); LC/MS (Method B): (electrospray+ve), m/z 390.1 (MH)⁺, t_{R}=3.10 min, UV₂₅₄=98%.

### 9-(6-Aminopyridin-3-yl)-1-(3-methoxyphenyl)benzo[h][1,6]naphthyridin-2(1H)-one

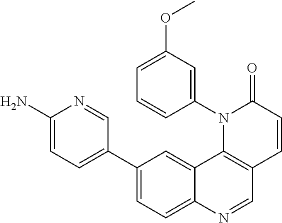 [SMILES: COc1cccc(-n2c(=O)ccc3cnc4ccc(-c5ccc(N)nc5)cc4c32)c1]

LC/MS (Method B): (electrospray+ve), m/z 395.1 (MH)⁺, t_{R}=3.20 min, UV₂₅₄=100%.

### 9-(6-Aminopyridin-3-yl)-1-(3-phenoxyphenyl)benzo[h][1,6]naphthyridin-2(1H)-one

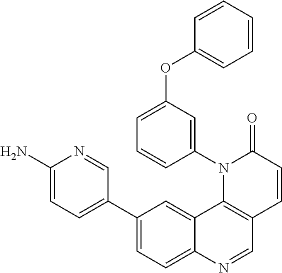 [SMILES: Nc1ccc(-c2ccc3ncc4ccc(=O)n(-c5cccc(Oc6ccccc6)c5)c4c3c2)cn1]

LC/MS (Method B): (electrospray+ve), m/z 457.1 (MH)⁺, t_{R}=3.81 min, UV₂₅₄=98%.

### 9-(6-Aminopyridin-3-yl)-1-(2-ethoxyphenyl)benzo[h][1,6]naphthyridin-2(1H)-one

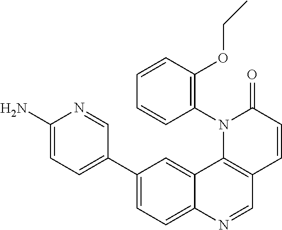 [SMILES: CCOc1ccccc1-n1c(=O)ccc2cnc3ccc(-c4ccc(N)nc4)cc3c21]

LC/MS (Method B): (electrospray+ve), m/z 409.1 (MH)⁺, t_{R}=3.33 min, UV₂₅₄=98%.

### 9-(6-Aminopyridin-3-yl)-1-(4-(piperazin-1-yl)-3-(trifluoromethyl)phenyl)benzo[h][1,6]naphthyridin-2(1H)-one

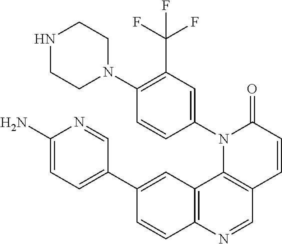 [SMILES: Nc1ccc(-c2ccc3ncc4ccc(=O)n(-c5ccc(N6CCNCC6)c(C(F)(F)F)c5)c4c3c2)cn1]

LC/MS (Method B): (electrospray+ve), m/z 517.2 (MH)⁺, t_{R}=3.18 min, UV₂₅₄=95%.

### 1-(4-((4-Acetylpiperazin-1-yl)sulfonyl)phenyl)-9-(6-aminopyridin-3-yl)benzo[h][1,6]naphthyridin-2(1H)-one

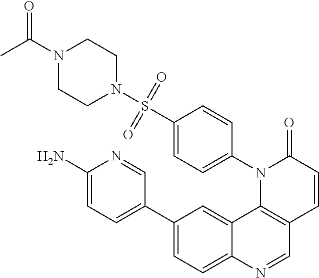 [SMILES: CC(=O)N1CCN(S(=O)(=O)c2ccc(-n3c(=O)ccc4cnc5ccc(-c6ccc(N)nc6)cc5c43)cc2)CC1]

LC/MS (Method B): (electrospray+ve), m/z 555.2 (MH)⁺, t_{R}=3.21 min, UV₂₅₄=95%.

### Example 7

This example demonstrates the synthesis of compounds in accordance with an embodiment of the invention.

### Ethyl 6-bromo-4-((4-(4-(tert-butoxycarbonyl)piperazin-1-yl)-3-(trifluoromethyl)phenyl)amino)quinoline-3-carboxylate (8)

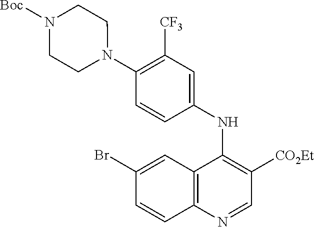 [SMILES: CCOC(=O)c1cnc2ccc(Br)cc2c1Nc1ccc(N2CCN(C(=O)OC(C)(C)C)CC2)c(C(F)(F)F)c1]

A mixture of tert-butyl 4-(4-amino-2-(trifluoromethyl)phenyl)piperazine-1-carboxylate (9, 0.691 g, 2.00 mmol) and ethyl 6-bromo-4-chloroquinoline-3-carboxylate (10, 0.629 g, 2.00 mmol) in 20 mL of THF was heated in a microwave for 15 min at 120° C. The reaction mixture was poured into 50 mL of EtOAc. The solution was washed twice with NaOH solution (1 N, 2×30 mL), dried over MgSO₄, filtered and concentrated. The crude product was purified by column chromatography on silica gel using 7-60% EtOAc in hexanes as eluent to give 8 (0.935 g, 75.0%) as a solid. ¹H NMR (400 MHz, CHLOROFORM-d) δ ppm 10.50 (s, 1H), 9.28 (s, 1H), 7.89 (d, J=9.00 Hz, 1H), 7.72 (dd, J=9.00, 1.96 Hz, 1H), 7.63 (d, J=2.35 Hz, 1H), 7.36 (d, J=2.35 Hz, 1H), 7.28 (d, J=9.00 Hz, 1H), 7.14 (dd, J=8.61, 2.35 Hz, 1H), 4.46 (q, J=7.30 Hz, 2H), 3.53-3.62 (m, 4H), 2.83-2.91 (m, 4H), 1.49 (s, 9H), 1.47 (t, J=7.30 Hz, 3H); LC/MS: (electrospray+ve), m/z 623.1 (MH)⁺, _{tR}=5.90 min, U_{V254}=100%.

### tert-Butyl 4-(4-((6-bromo-3-(hydroxymethyl)quinolin-4-yl)amino)-2-(trifluoromethyl)phenyl)piperazine-1-carboxylate (11)

To a solution of ethyl 6-bromo-4-((4-(4-(tert-butoxycarbonyl)piperazin-1-yl)-3-(trifluoromethyl)phenyl)amino)quinoline-3-carboxylate (8, 1.24 g, 1.99 mmol) in 200 mL of ethanol was added NaBH₄ (0.752 g, 19.9 mmol) at room temperature. After stirring for 4 h, the mixture was poured into 300 mL of EtOAc. The solution was washed with water (3×200 mL). The organic layer was dried over MgSO₄, filtered, and concentrated. The residue was purified by column chromatography through a C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 11 (342 mg, 0.59 mmol, 30%). ¹H NMR (400 MHz, DMSO-d₆) δ 9.01 (s, 1H), 8.74 (s, 1H), 8.22 (d, J=2.2 Hz, 1H), 7.96 (d, J=8.9 Hz, 1H), 7.84 (dd, J=8.9, 2.2 Hz, 1H), 7.40 (d, J=8.7 Hz, 1H), 7.03 (d, J=2.7 Hz, 1H), 6.80 (dd, J=8.7, 2.7 Hz, 1H), 5.41 (t, J=5.4 Hz, 1H), 4.43 (d, J=5.5 Hz, 2H), 3.28-3.36 (m, 4H), 2.70-2.76 (m, 4H), 1.41 (s, 9H); LC/MS: (electrospray+ve), m/z 581.1 (MH)⁺, t_{R}=5.23 min, UV₂₅₄>95%.

### tert-Butyl 4-(4-((6-bromo-3-formylquinolin-4-yl)amino)-2-(trifluoromethyl)phenyl)piperazine-1-carboxylate (12)

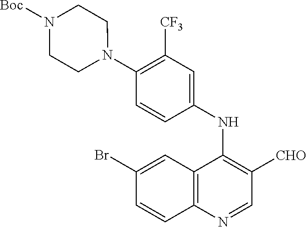 [SMILES: CC(C)(C)OC(=O)N1CCN(c2ccc(Nc3c(C=O)cnc4ccc(Br)cc34)cc2C(F)(F)F)CC1]

To a solution of tert-butyl 4-(4-((6-bromo-3-(hydroxymethyl)quinolin-4-yl)amino)-2-(trifluoromethyl)phenyl)piperazine-1-carboxylate (11, 335 mg, 0.576 mmol) in 30 mL of DCM was added Dess-Martin reagent (367 mg, 0.864 mmol). After stirring at room temperature for 2 h, the mixture was poured into 100 mL of ethyl acetate, washed with NaOH solution (1.0 N, 3×50 mL). The organic layer was dried over MgS_{O4}, filtered, and concentrated. The crude product was purified on silica gel using 2-10% MeOH in DCM as eluent to give 12 (257 mg, 77%) as a solid. ¹H NMR (400 MHz, DMSO-_{d6}) δ 10.38 (s, 1H), 10.01 (s, 1H), 8.91 (s, 1H), 8.16 (d, J=2.1 Hz, 1H), 7.96-7.83 (m, 2H), 7.57-7.46 (m, 2H), 7.40 (dd, J=8.7, 2.6 Hz, 1H), 3.40-3.46 (m, 4H), 2.78-2.82 (m, 4H), 1.42 (s, 9H); LC/MS: (electrospray+ve), m/z 579.1 (MH)⁺, _{tR}=5.54 min, U_{V254}=98%.

### tert-Butyl 4-(4-(9-bromo-2-oxobenzo[h][1,6]naphthyridin-1(2H)-yl)-2-(trifluoromethyl)phenyl)piperazine-1-carboxylate (13)

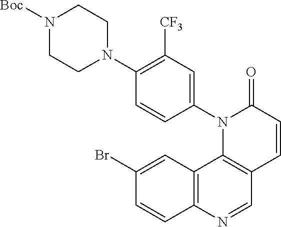 [SMILES: CC(C)(C)OC(=O)N1CCN(c2ccc(-n3c(=O)ccc4cnc5ccc(Br)cc5c43)cc2C(F)(F)F)CC1]

To a solution of tert-butyl 4-(4-((6-bromo-3-formylquinolin-4-yl)amino)-2-(trifluoromethyl)phenyl)piperazine-1-carboxylate (12, 251 mg, 0.434 mmol) and ethyl triethyl phosphonoacetate (146 mg, 0.650 mmol) in 3 mL of EtOH was added potassium carbonate (299 mg, 2.166 mmol). After heating at 150° C. for 15 min in a microwave, the reaction mixture was poured into 50 mL of EtOAc. The solution was washed with NaOH solution (1.0 N, 3×30 mL). The organic layer was dried over MgSO₄, filtered, and concentrated. The crude product was purified by column chromatography on silica gel using 2-10% MeOH in DCM as eluent to give 13 (205 mg, 0.340 mmol, 78%) as a solid. ¹H NMR (400 MHz, DMSO-d₆) δ 9.18 (s, 1H), 8.32 (d, J=9.5 Hz, 111), 8.00-7.84 (m, 3H), 7.76-7.83 (m, 2H), 6.96 (d, J=9.4 Hz, 1H), 6.54 (d, J=2.1 Hz, 1H), 3.48-3.53 (m, 4H), 2.96-3.32 (m, 4H), 1.44 (s, 9H); LC/MS: (electrospray+ve), m/z 603.1 (MH)⁺, t_{R}=6.46 min, UV₂₅₄=98%.

### tert-Butyl 4-(4-(9-(6-aminopyridin-3-yl)-2-oxobenzo[h][1,6]naphthyridin-1(2H)-yl)-2-(trifluoromethyl)phenyl)piperazine-1-carboxylate (14a)

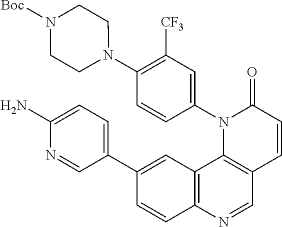 [SMILES: CC(C)(C)OC(=O)N1CCN(c2ccc(-n3c(=O)ccc4cnc5ccc(-c6ccc(N)nc6)cc5c43)cc2C(F)(F)F)CC1]

A mixture of 5-(4,4,5,5-tetramethyl-1,3,2-dioxaborolan-2-yl)pyridin-2-amine (58.4 mg, 0.265 mmol), tert-butyl 4-(4-(9-bromo-2-oxobenzo[h][1,6]naphthyridin-1(2H)-yl)-2-(trifluoromethyl)phenyl)piperazine-1-carboxylate (13, 80.1 mg, 0.133 mmol) and Pd(Ph₃P)₄ (15.3 mg, 0.013 mmol) in 3 mL of DMF and 0.6 mL of saturated Na₂CO₃ aqueous solution was heated in a microwave for 10 min at 150° C. The reaction mixture was filtered through a plug of celite and the filtrate was purified by column chromatography through C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 14a (64.2 mg 78%). ¹H NMR (400 MHz, DMSO-d₆) δ 9.09 (s, 1H), 8.30 (d, J=9.5 Hz, 1H), 8.05 (d, J=8.7 Hz, 1H), 8.00-7.96 (m, 2H), 7.81-7.67 (m, 3H), 7.10 (dd, J=8.6, 2.6 Hz, 1H), 6.94 (d, J=1.9 Hz, 1H), 6.91 (d, J=9.4 Hz, 1H), 6.41 (dd, J=8.6, 0.8 Hz, 1H), 6.18 (s, 2H), 3.47-3.53 (m, 4H), 2.86-2.94 (m, 4H), 1.45 (s, 9H). LC/MS: (electrospray+ve), m/z 617.2 (MH)⁺, t_{R}=4.63 min, UV₂₅₄=95%.

### tert-Butyl 4-(4-((6-bromo-3-formylquinolin-4-yl)amino)-2-(trifluoromethyl)phenyl)piperazine-1-carboxylate (14b)

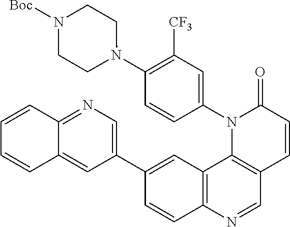 [SMILES: CC(C)(C)OC(=O)N1CCN(c2ccc(-n3c(=O)ccc4cnc5ccc(-c6cnc7ccccc7c6)cc5c43)cc2C(F)(F)F)CC1]

A mixture of 3-quinolineboronic acid (46.2 mg, 0.267 mmol), tert-butyl 4-(4-(9-bromo-2-oxobenzo[h][1,6]naphthyridin-1(2H)-yl)-2-(trifluoromethyl)phenyl)piperazine-1-carboxylate (13, 80.5 mg, 0.133 mmol) and Pd(Ph₃P)₄ (15.4 mg, 0.013 mmol) in 3 mL of DMF and 0.6 mL of saturated Na₂CO₃ aqueous solution was heated in a microwave for 10 min at 150° C. The reaction mixture was filtered through a plug of celite and the filtrate was purified by column chromatography through C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 14b (56.8 mg 65.3%). ¹H NMR (400 MHz, DMSO-d₆) δ 9.25 (s, 1H), 8.61-8.66 (m, 1H), 8.40 (d, J=9.5 Hz, 1H), 8.34-8.14 (m, 3H), 8.10-7.99 (m, 3H), 7.90-7.69 (m, 4H), 7.13 (d, J=1.8 Hz, 1H), 7.01 (d, J=9.4 Hz, 1H), 3.33-3.38 (m, 2H), 3.21-3.26 (m, 2H), 2.67-2.56 (m, 2H), 2.46-2.52 (m, 2H), 1.47 (s, 9H); LC/MS: (electrospray+ve), m/z 652.2 (MH)⁺, t_{R}=5.96 min, UV₂₅₄=95%.

### tert-Butyl 4-(4-((6-bromo-3-formylquinolin-4-yl)amino)-2-(trifluoromethyl)phenyl)piperazine-1-carboxylate (15c, WWH30)

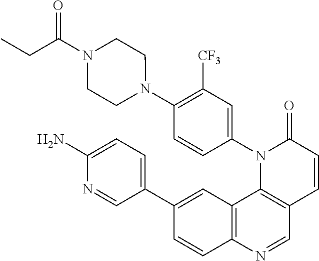 [SMILES: CCC(=O)N1CCN(c2ccc(-n3c(=O)ccc4cnc5ccc(-c6ccc(N)nc6)cc5c43)cc2C(F)(F)F)CC1]

To a solution of tert-butyl 4-(4-(9-(6-aminopyridin-3-yl)-2-oxobenzo[h][1,6]naphthyridin-1(2H)-yl)-2-(trifluoromethyl)phenyl)piperazine-1-carboxylate (14a, 23.5 mg, 0.038 mmol) in 3 mL of dichloroethane was added 1 mL of TFA. The mixture was stirred at room temperature for 2 h. The solvent was removed and the residue was dissolved in 3 mL of MeOH. To this solution was added triethylamine (19.3 mg, 0.191 mmol) and propionyl chloride (7.1 mg, 0.076 mmol). The resulted mixture was stirred at room temperature for 4 h. The crude mixture was purified by column chromatography through C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 15c (16.7 mg, 77%). ¹H NMR (400 MHz, DMSO-d₆) δ9.19 (s, 1H), 8.34 (d, J=9.5 Hz, 1H), 8.13 (d, J=8.7 Hz, 1H), 8.07 (d, J=2.2 Hz, 1H), 8.03-7.93 (m, 4H), 7.78-7.68 (m, 2H), 7.37 (dd, J=9.3, 2.3 Hz, 1H), 7.00-6.84 (m, 3H), 3.63-3.80 (m, 2H), 3.47-3.57 (m, 2H), 2.87-3.00 (m, 2H), 2.83-2.67 (m, 2H), 2.39 (q, J=7.4 Hz, 2H), 1.04 (t, J=7.4 Hz, 3H). LC/MS: (electrospray+ve), m/z 573.2 (MH)⁺, t_{R}=3.85 min, UV₂₅₄=100%.

### tert-Butyl (15-(4-(4-(9-(6-aminopyridin-3-yl)-2-oxobenzo[h][1,6]naphthyridin-1(2H)-yl)-2-(trifluoromethyl)phenyl)piperazin-1-yl)-15-oxo-3,6,9,12-tetraoxapentadecyl)carbamate (15a)

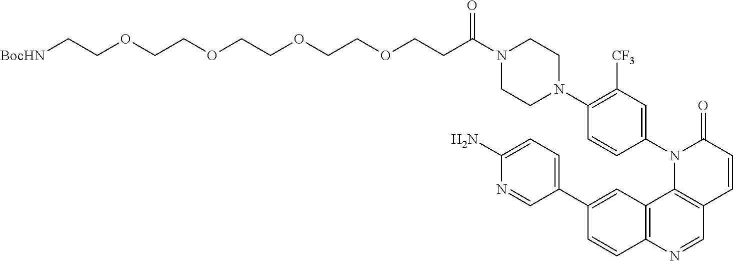 [SMILES: CC(C)(C)OC(=O)NCCOCCOCCOCCOCCC(=O)N1CCN(c2ccc(-n3c(=O)ccc4cnc5ccc(-c6ccc(N)nc6)cc5c43)cc2C(F)(F)F)CC1]

To a solution of tert-butyl 4-(4-(9-(6-aminopyridin-3-yl)-2-oxobenzo[h][1,6]naphthyridin-1(2H)-yl)-2-(trifluoromethyl)phenyl)piperazine-1-carboxylate (14a, 57.2 mg, 0.093 mmol) in 3 mL of dichloroethane was added 1 mL of TFA. The mixture was stirred at room temperature for 2 h. The solvent was removed under vacuum and the residue was dissolved in 5 mL of DMF. To this solution was added triethylamine (40 mg, 0.40 mmol), 2,2-dimethyl-4-oxo-3,8,11,14,17-pentaoxa-5-azaicosan-20-oic acid (35.6 mg, 0.097 mmol), and HATU (51.7 mg, 0.111 mmol). The resulted mixture was stirred at room temperature for 6 h. The crude mixture was purified by column chromatography through C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 15a (46.1 mg, 58%). ¹H NMR (400 MHz, DMSO-d₆) δ 9.09 (s, 1H), 8.30 (d, J=9.4 Hz, 1H), 8.05 (d, J=8.7 Hz, 1H), 7.99-7.87 (m, 2H), 7.79-7.69 (m, 3H), 7.14 (dd, J=8.6, 2.6 Hz, 1H), 6.98-6.88 (m, 2H), 6.74 (t, J=5.9 Hz, 1H), 6.42 (dd, J=8.6, 0.8 Hz, 1H), 6.19 (s, 2H), 3.61-3.70 (m, 5H), 3.45-3.55 (m, 10H), 3.23-3.38 (m, 5H), 2.87-3.07 (m, 5H), 2.63-2.70 (m, 3H), 1.36 (s, 9H); LC/MS: (electrospray+ve), m/z 864.3 (MH)⁺, t_{R}=4.40 min, UV₂₅₄=100%.

### tert-Butyl (15-oxo-15-(4-(4-(2-oxo-9-(quinolin-3-yl)benzo[h][1,6]naphthyridin-1(2H)-yl)-2-(trifluoromethyl)phenyl)piperazin-1-yl)-3,6,9,12-tetraoxapentadecyl)carbamate (15b)

To a solution of tert-butyl 4-(4-((6-bromo-3-formylquinolin-4-yl)amino)-2-(trifluoromethyl)phenyl)piperazine-1-carboxylate (14b, 50.6 mg, 0.078 mmol) in 3 mL of dichloroethane was added 1 mL of TFA. The mixture was stirred at room temperature for 2 h. The solvent was removed under vacuum and the residue was dissolved in 5 mL of DMF. To this solution was added triethylamine (19.3 mg, 0.191 mmol), 2,2-dimethyl-4-oxo-3,8,11,14,17-pentaoxa-5-azaicosan-20-oic acid (29.8 mg, 0.082 mmol), and HATU (35.4 mg, 0.093 mmol). The resulted mixture was stirred at room temperature for 6 h. The crude mixture was purified by column chromatography through C18 column using 5-100% ACN (containing 0.1% TFA)/water (containing 0.1% TFA) as eluent. The combined pure fractions were neutralized using 1 N NaOH solution, extracted with ethyl acetate (3×50 mL). The organic layer was dried over MgSO₄, filtered, and concentrated to give 15a (42.2 mg, 60%). ¹H NMR (400 MHz, DMSO-d₆) δ 9.21 (s, 1H), 8.59 (d, J=2.3 Hz, 1H), 8.36 (d, J=9.5 Hz, 1H), 8.28 (dd, J=2.3, 0.8 Hz, 1H), 8.25-8.13 (m, 2H), 8.10-7.96 (m, 3H), 7.63-7.90 (m, 4H), 7.13 (d, J=1.7 Hz, 1H), 6.97 (d, J=9.4 Hz, 1H), 6.73 (t, J=5.5 Hz, 1H), 3.71-3.43 (m, 19H), 3.35 (t, J=6.0 Hz, 1H), 3.04 (q, J=6.0 Hz, 2H), 2.67-2.74 (m, 3H), 2.51-2.62 (m, 3H), 1.36 (s, 9H); LC/MS: (electrospray+ve), m/z 899.3 (MH)⁺, t_{R}=5.43 min, UV₂₅₄>95%.

Polymer linked Torin 2 (16a)

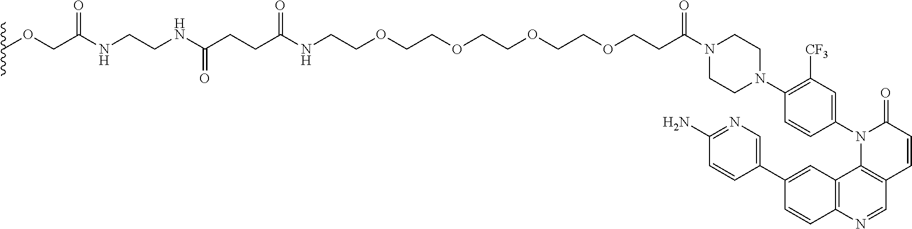 [SMILES: *OCC(=O)NCCNC(=O)CCC(=O)NCCOCCOCCOCCOCCC(=O)N1CCN(c2ccc(-n3c(=O)ccc4cnc5ccc(-c6ccc(N)nc6)cc5c43)cc2C(F)(F)F)CC1]

To a mixture of Affi-Gel 10 (Bio-Rad Laboratories, cat. no. 153-6046), 3 mL gel, 45 μmol) and 15a (12.9 mg, 15 μmole) in 10 mL of DMSO was added triethylamine (150 μmol). The mixture was shaken at room temperature for 6 h (15a disappeared from solution based on LC-MS analysis). Then ethanolamine (300 μmol) was added and the resulted mixture was shaken at room temperature for overnight. After washing with DMSO and PBS, the polymer linked Torin 2 (16a) was stored in PBS (containing 0.1% sodium azide) at 4° C.

Polymer linked Torin 1 (16b) was prepared in a similar manner as polymer linked Torin 2.

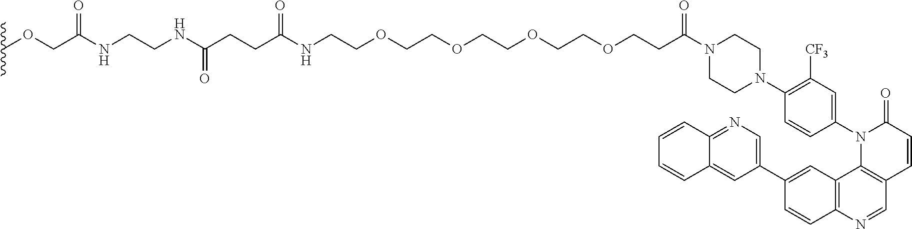 [SMILES: *OCC(=O)NCCNC(=O)CCC(=O)NCCOCCOCCOCCOCCC(=O)N1CCN(c2ccc(-n3c(=O)ccc4cnc5ccc(-c6cnc7ccccc7c6)cc5c43)cc2C(F)(F)F)CC1]

### Example 8

This example demonstrates an In vitro drug activity on gametocytes.

Stage III-V gametocytes were enriched with treatment with 50 mM N-acetylglucosamine (NAG) and Percoll density gradient centrifugation as described previously¹. Briefly, 2.5 μl/well incomplete medium was dispensed into each well of 1,536-well plates using the Multidrop Combi followed by 23 nl compound transfer using the NX-TR Pintool (WAKO Scientific Solutions, San Diego, Calif.). Then, 2.5 μl/well of gametocytes was dispensed with a seeding density of 20,000 cells/well using the Multidrop Combi. The assay plates were incubated for 72 h at 37° C. with 5% CO₂. After addition of 5 μl/well of 2× AlamarBlue dye (Life Technologies, cat. no. DAL1100), the plates were incubated for 24 h at 37° C. with 5% CO₂ and then were read in a fluorescence detection mode (Ex=525 nm, Em=598 nm) on a ViewLux plate reader (PerkinElmer).

### Example 9

This example demonstrates In vitro drug activity on asexual parasites in accordance with an embodiment of the invention.

Asexual parasites of P. falciparum strain 3D7 were cultured as described previously (Trager, W. et al., J. Parasitol. 2005, 91(3): 484-486). Drug activity on asexual stage parasites was tested using a SYBR Green assay as described previously (Eastman, R. T. et al., Antimicrob. Agents Chemother. 2013, 57(1): 425-435; Smilkstein, M. et al., Antimicrob. Agents Chemother. 2004, 48(5): 1803-1806). Briefly, parasites were diluted to 0.5% parasitemia in complete culture medium with 2% hematocrit and drugs diluted in DMSO (≦0.5%) and were loaded into a 96-well plate (200 μl/well). No drug and RBC alone wells were included as positive and background controls, respectively, and each testing condition was examined in duplicated. After 72 h incubation under the standard culture condition and a freeze-thaw lysis step at −80° C. and room temperature, 100 μl/well of lysis buffer containing SYBR Green I was added to the parasite culture and incubated for 30 min at room temperature. The fluorescence of each well was measured at 520 nm following excitation at 490 nm using a FLUOstar Optima microplate reader (BMG Labtech).

### Example 10

This example demonstrates the efficacy of NVP-AUY922 and Alvespimycin on gametocyte transmission from host to mosquitoes in a mouse model in accordance with an embodiment of the invention.

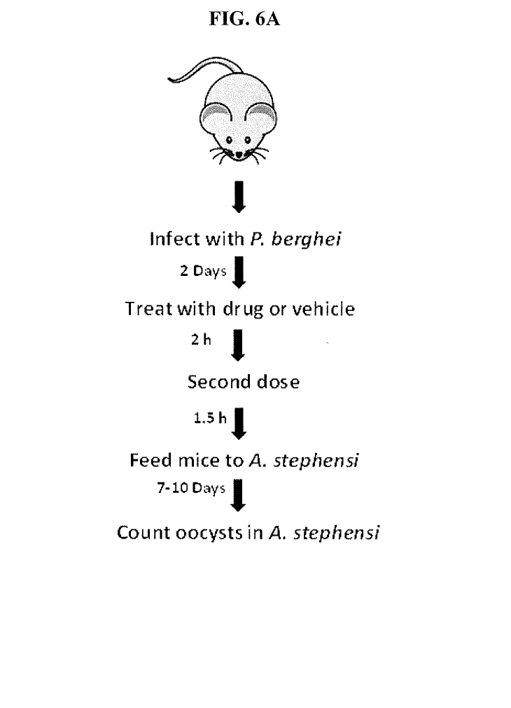

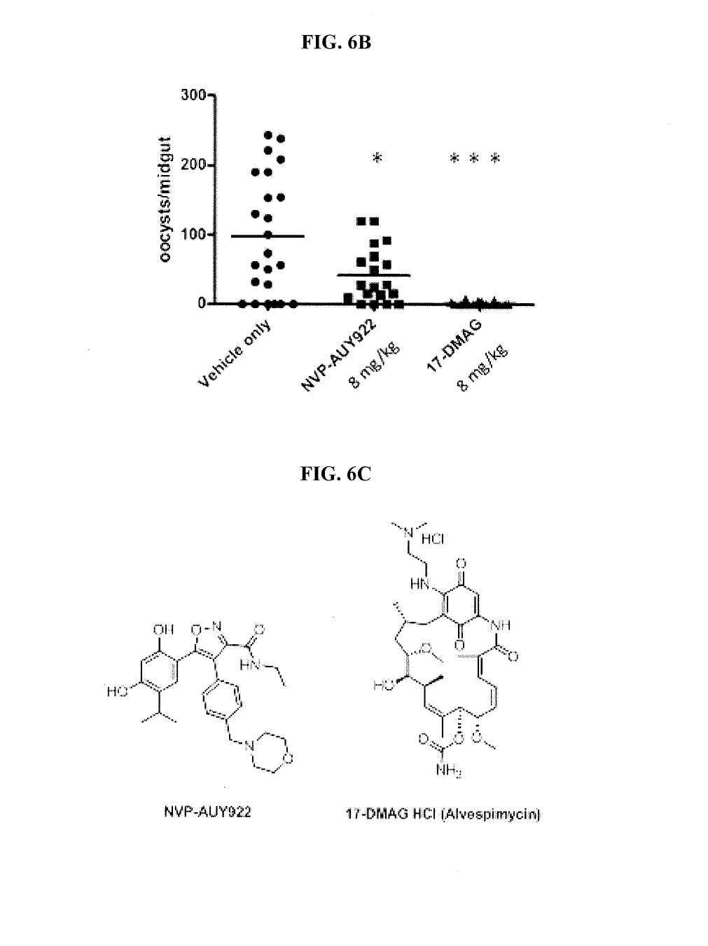

The experiment described in Example 4 was conducted using NVP-AUY922 and Alvespimycin in a two dose protocol at 8 mg/kg as test compounds. The protocol is depicted graphically in FIG. 6A. The oocyte number for vehicle, NVP-AUY922, and Alvespimycin-treated mice are depicted in FIG. 6B. The structures of NVP-AUY922 and Alvespimycin are depicted in FIG. 6C.

### Example 11

This example demonstrates the synthesis of compounds in accordance with an embodiment of the invention.

A reaction scheme for the synthesis of compounds 13 and 14 is as follows:

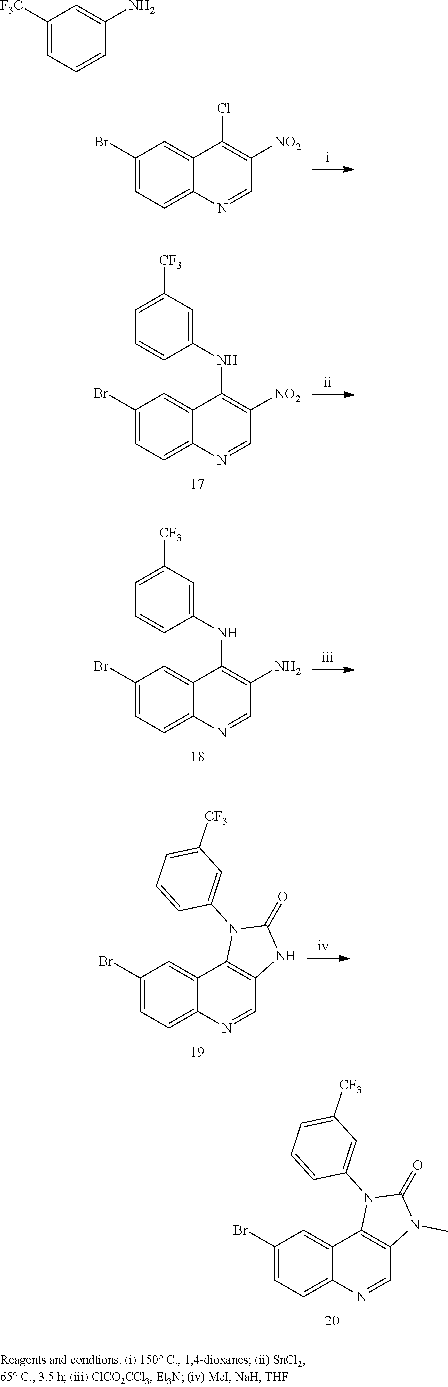

### 6-Bromo-3-nitro-N-(3-(trifluoromethyl)phenyl)quinolin-4-amine

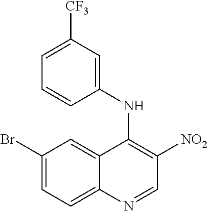 [SMILES: O=[N+]([O-])c1cnc2ccc(Br)cc2c1Nc1cccc(C(F)(F)F)c1]

A solution of 6-bromo-4-chloro-3-nitroquinoline (1 g, 3.48 mmol) in 1,4-dioxane (11 mL) at room temperature was treated with 3-(trifluoromethyl)aniline (0.434 mL, 3.48 mmol). The mixture was allowed to heat at 150° C. for 2 hours and monitored via LC-MS for completion. The reaction mixture was treated with brine and extracted with ethyl acetate (3×). The organic layers were collected, dried, filtered, and concentrated. Purification by SiO₂ chromatography (0-50% Hex/EA) afforded the desired product as an off-white solid (1.32 g, 3.2 mmol, 92%). LC/MS (Method A): (electrospray+ve), m/z 412.1 (MH)⁺, t_{R}=3.706, UV₂₅₄=100%.

### 6-Bromo-N4-(3-(trifluoromethyl)phenyl)76quinoline-3,4-diamine

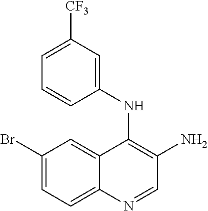 [SMILES: Nc1cnc2ccc(Br)cc2c1Nc1cccc(C(F)(F)F)c1]

A solution of 6-bromo-N4-(3-(trifluoromethyl)phenyl)quinoline-3,4-diamine (1.3 0 g, 3.15 mmol) in ethyl acetate (12 mL) was treated with tin (II) chloride (3.56 g, 15.7 mmol) at room temperature. The mixture was heated at 65° C. for 3.5 hours and monitored via LC-MS for completion. The reaction mixture was cooled to room temperature and treated with 10N NaOH (20 mL). The mixture was filtered over Celite, and the filtrate was diluted with deionized water and extracted with ethyl acetate (3×). The organic layers were collected, dried, filtered and concentrated. Purification via SiO₂ chromatography (0-10% DCM-MeOH) afforded the desired product as a yellow solid (1.15 g, 3.01 mmol, 95%). ¹H NMR (400 MHz, DMSO-d₆) δ 8.63 (s, 1H), 8.21 (s, 1H), 7.83 (d, J=2.2 Hz, 1H), 7.77 (d, J=8.9 Hz, 1H), 7.46 (dd, J=8.9, 2.1 Hz, 1H), 7.32 (t, J=8.0 Hz, 1H), 6.99 (d, J=7.8 Hz, 1H), 6.82 (s, 1H), 6.66 (dd, J=8.2, 2.2 Hz, 1H), 5.55 (s, 2H); LC/MS (Method A): (electrospray+ve), m/z 383.1 (MH)⁺, t_{R}=3.112, UV₂₅₄=100%.

### 8-Bromo-1-(3-(trifluoromethyl)phenyl)-1H-imidazo[4,5-c]quinolin-2(3H)-one

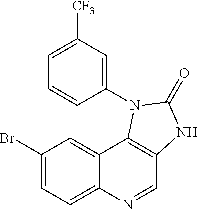 [SMILES: O=c1[nH]c2cnc3ccc(Br)cc3c2n1-c1cccc(C(F)(F)F)c1]

A solution of 6-bromo-N4-(3-(trifluoromethyl)phenyl)quinoline-3,4-diamine (1.15 g, 3.01 mmol) and triethylamine (0.586 mL, 4.21 mmol) in dichloromethane (35 mL) was cooled to 0° C. To the solution, trichloromethyl chloroformate (0.465 mL, 3.85 mmol) in a solution of dichloromethane (35 mL) was added. The mixture stirred for 30 minutes at 0° C. and monitored via LC-MS for completion. The reaction mixture was quenched with brine and extracted with dichloromethane (3×). The organic layers were combined, dried, filtered, and concentrated. Purification via SiO₂ chromatography (0-10% DCM-MeOH) afforded the desired product as a pale yellow solid (0.821 g, 2.012 mmol, 66.9%). ¹H NMR (400 MHz, DMSO-d₆) δ 11.89 (s, 1H), 8.80 (s, 1H), 8.15-8.09 (m, 1H), 8.04 (dt, J=8.2, 1.5 Hz, 1H), 7.98 (dt, J=8.0, 1.7 Hz, 1H), 7.94 (dd, J=8.6, 7.1 Hz, 2H), 7.65 (dd, J=9.0, 2.2 Hz, 1H), 7.02 (d, J=2.2 Hz, 1H); LC/MS (Method B): (electrospray+ve), m/z 408.0 (MH)⁺, t_{R}=4.458, UV₂₅₄=100%.

### 8-Bromo-3-methyl-1-(3-(trifluoromethyl)phenyl)-1H-imidazo[4,5-c]quinolin-2(3H)-one

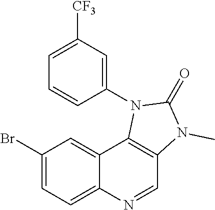 [SMILES: Cn1c(=O)n(-c2cccc(C(F)(F)F)c2)c2c3cc(Br)ccc3ncc21]

A room temperature solution of 8-bromo-1-(3-(trifluoromethyl)phenyl)-1H-imidazo[4,5-c]quinolin-2(3H)-one (0.40 g, 0.98 mmol) in tetrahydrofuran (7 mL) under N₂ was treated with iodomethane (0.073 mL, 1.176 mmol) followed by sodium hydride (70.6 mg, 2.94 mmol). The reaction mixture was allowed to stir at room temperature overnight and monitored via LC-MS. The reaction was quenched with ammonium chloride and extracted with dichloromethane. The organic layers were combined, dried, filtered, and concentrated. Purification via SiO₂ chromatography (0-10% DCM-MeOH) afforded the desired product as an orange solid (0.315 g, 0.747 mmol, 76%). ¹H NMR (400 MHz, DMSO-d₆) δ 9.05 (s, 1H), 8.11 (d, J=2.2 Hz, 1H), 8.05 (d, J=7.8 Hz, 114), 7.97 (dd, J=8.7, 3.4 Hz, 2H), 7.92 (t, J=7.9 Hz, 1H), 7.66 (dd, J=9.0, 2.2 Hz, 1H), 7.02 (d, J=2.2 Hz, 1H), 3.58 (s, 3H); LC/MS (Method B): (electrospray+ve), m/z 421.9 (MH)⁺, t_{R}=3.060, UV₂₅₄=100%

General Procedure for the Synthesis of 21:

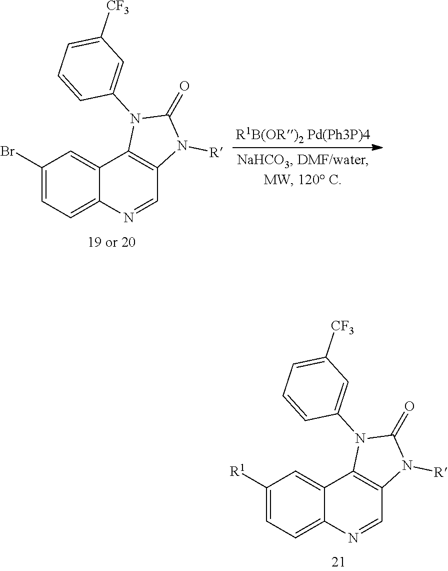

A mixture of 19 or 20 (200 umole, 1.0 equiv), boronic acid or boronic acid pinacol ester (2.0 equiv), tetrakis(triphenylphosphine)palladium (0.05 equiv), DMF (1.5 mL) and saturated NaHCO₃ aqueous solution (0.5 mL) was charged in a microwave vial. Nitrogen was bubbled through the mixture for 3 min. The vial was capped and heated in a microwave at 120-150° C. for 40 min. The reaction mixture was filtered through a plug of Celite and the filtrate was purified by reverse phase column chromatography using acetonitrile (containing 0.1% TFA)/water (containing 0.1% TFA) as an eluent to give 21.

### 8-(4-Aminophenyl)-1-(3-(trifluoromethyl)phenyl)-1H-imidazo[4,5-c]quinolin-2(3H)-one

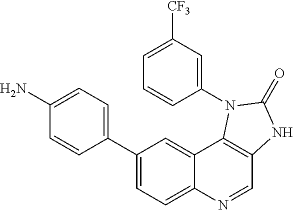 [SMILES: Nc1ccc(-c2ccc3ncc4[nH]c(=O)n(-c5cccc(C(F)(F)F)c5)c4c3c2)cc1]

¹H NMR (400 MHz, DMSO-d₆) δ 12.20 (s, 1H), 8.91 (s, 1H), 8.25 (d, J=2.0 Hz, 1H), 8.12-8.09 (m, 1H), 8.07 (d, J=9.1 Hz, 1H), 8.06-8.02 (m, 1H), 7.99-7.94 (m, 2H), 7.04-7.01 (m, 2H), 7.00 (d, J=1.9 Hz, 1H), 6.60-6.55 (m, 2H); LC/MS (Method B): (electrospray+ve), m/z 421.1 (MH)⁺, t_{R}=3.575, UV₂₅₄=100%

### 8-(6-Methylpyridin-3-yl)-1-(3-(trifluoromethyl)phenyl)-1H-imidazo[4,5-c]quinolin-2(3H)-one

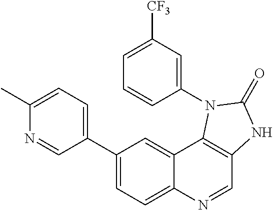 [SMILES: Cc1ccc(-c2ccc3ncc4[nH]c(=O)n(-c5cccc(C(F)(F)F)c5)c4c3c2)cn1]

¹H NMR (400 MHz, DMSO-d₆) δ 12.11 (s, 1H), 8.92 (s, 1H), 8.45 (d, J=2.4 Hz, 1H), 8.22 (d, J=2.0 Hz, 1H), 8.17 (d, J=8.9 Hz, 1H), 8.07 (d, J=8.0 Hz, 1H), 8.04 (d, J=1.9 Hz, 1H), 8.01 (dd, J=8.9, 2.0 Hz, 1H), 7.96 (d, J=7.9 Hz, 1H), 7.74 (dd, J=8.1, 2.5 Hz, 1H), 7.39 (d, J=8.2 Hz, 1H), 7.18 (d, J=2.1 Hz, 1H), 2.51 (s, 3H); LC/MS (Method B): (electrospray+ve), m/z 421.1 (MH)⁺, t_{R}=3.296, UV₂₅₄=100%

### N-(5-(2-Oxo-1-(3-(trifluoromethyl)phenyl)-2,3-dihydro-1H-imidazo[4,5-c]quinolin-8-yl)pyridin-2-yl)acetamide

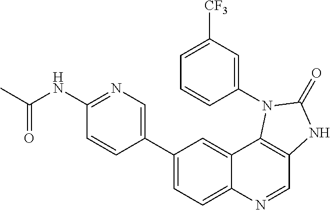 [SMILES: CC(=O)Nc1ccc(-c2ccc3ncc4[nH]c(=O)n(-c5cccc(C(F)(F)F)c5)c4c3c2)cn1]

¹H NMR (400 MHz, DMSO-d₆) δ 10.64 (s, 1H), 8.77 (s, 1H), 8.20 (d, J=2.2 Hz, 1H), 8.18 (dd, J=2.6, 0.8 Hz, 1H), 8.09 (d, J=3.8 Hz, 1H), 8.07 (d, J=3.8 Hz, 1H), 8.07-8.00 (m, 3H), 7.94 (d, J=7.9 Hz, 1H), 7.90 (dd, J=8.9, 2.0 Hz, 1H), 7.78 (dd, J=8.7, 2.6 Hz, 1H), 7.11 (d, J=2.0 Hz, 1H), 2.08 (s, 3H); LC/MS (Method B): (electrospray+ve), m/z 464.1 (MH)⁺, t_{R}=3.710, UV₂₅₄=100%

### 8-(4-Hydroxyphenyl)-1-(3-(trifluoromethyl)phenyl)-1H-imidazo[4,5-c]quinolin-2(3H)-one

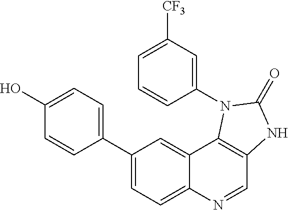 [SMILES: O=c1[nH]c2cnc3ccc(-c4ccc(O)cc4)cc3c2n1-c1cccc(C(F)(F)F)c1]

¹H NMR (400 MHz, DMSO-d₆) δ 11.75 (s, 1H), 9.61 (s, 1H), 8.72 (s, 1H), 8.20 (s, 1H), 8.08-7.96 (m, 3H), 7.92 (t, J=7.8 Hz, 1H), 7.79 (dd, J=8.9, 2.1 Hz, 1H), 7.17-7.10 (m, 2H), 7.02 (d, J=2.0 Hz, 1H), 6.76-6.69 (m, 2H); LC/MS (Method B): (electrospray+ve), m/z 422.1 (MH)⁺, t_{R}=3.910, UV₂₅₄=100%

### 8-(3-Aminophenyl)-1-(3-(trifluoromethyl)phenyl)-1H-imidazo[4,5-c]quinolin-2(3H)-one

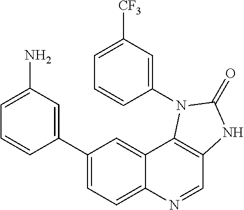 [SMILES: Nc1cccc(-c2ccc3ncc4[nH]c(=O)n(-c5cccc(C(F)(F)F)c5)c4c3c2)c1]

¹H NMR (400 MHz, DMSO-d₆) δ 11.76 (s, 1H), 8.73 (s, 1H), 8.16 (s, 1H), 8.03 (d, J=8.8 Hz, 1H), 8.02-7.98 (m, 2H), 7.93 (t, J=7.8 Hz, 1H), 7.72 (dd, J=8.8, 2.1 Hz, 1H), 7.09 (d, J=2.0 Hz, 1H), 6.95 (t, J=7.8 Hz, 1H), 6.62 (t, J=2.0 Hz, 1H), 6.51 (dd, J=7.2, 2.3 Hz, 1H), 6.35-6.32 (m, 1H), 5.07 (s, 2H); LC/MS (Method B): (electrospray+ve), m/z 421.1 (MH)⁺, t_{R}=3.630, UV₂₅₄=100%

### 8-(6-Aminopyridin-3-yl)-1-(3-(trifluoromethyl)phenyl)-1H-imidazo[4,5-c]quinolin-2(3H)-one

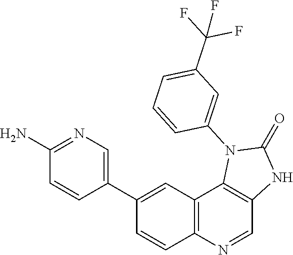 [SMILES: Nc1ccc(-c2ccc3ncc4[nH]c(=O)n(-c5cccc(C(F)(F)F)c5)c4c3c2)cn1]

LC/MS (Method B): (electrospray+ve), m/z 421.8 (MH)⁺, t_{R}=3.09, UV₂₅₄=100%

### 8-(Pyridin-4-yl)-1-(3-(trifluoromethyl)phenyl)-1H-imidazo[4,5-c]quinolin-2(3H)-one

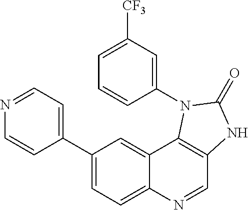 [SMILES: O=c1[nH]c2cnc3ccc(-c4ccncc4)cc3c2n1-c1cccc(C(F)(F)F)c1]

¹H NMR (400 MHz, DMSO-d₆) δ 11.86 (s, 1H), 8.81 (s, 1H), 8.56-8.53 (m, 2H), 8.22 (s, 1H), 8.13 (d, J=8.9 Hz, 1H), 8.05 (t, J=9.6 Hz, 2H), 7.95 (d, J=8.3 Hz, 2H), 7.31 7.28 (m, 2H), 7.25 (d, J=2.1 Hz, 1H); LC/MS (Method B): (electrospray+ve), m/z 407.1 (MH)⁺, t_{R}=3.116, UV₂₅₄=100%

### 8-(Pyridin-3-yl)-1-(3-(trifluoromethyl)phenyl)-1H-imidazo[4,5-c]quinolin-2(3H)-one

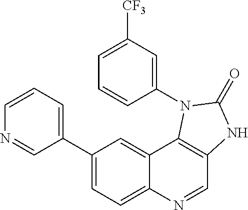 [SMILES: O=c1[nH]c2cnc3ccc(-c4cccnc4)cc3c2n1-c1cccc(C(F)(F)F)c1]

¹H NMR (400 MHz, DMSO-d₆) δ 11.93 (s, 1H), 8.84 (s, 1H), 8.53 (d, J=4.3 Hz, 1H), 8.51 (d, J=2.6 Hz, 1H), 8.20 (s, 1H), 8.14 (d, J=8.9 Hz, 1H), 8.05 (s, 1H), 8.02 (s, 1H), 7.97-7.94 (m, 1H), 7.94-7.90 (m, 1H), 7.75 (dd, J=7.9, 2.2 Hz, 1H), 7.42 (dd, J=7.9, 4.7 Hz, 1H), 7.16 (d, J=2.0 Hz, 1H); LC/MS (Method B): (electrospray+ve), m/z 407.1 (MH)⁺, t_{R}=3.306, UV₂₅₄=100%

### 8-(1H-Benzo[d]imidazol-5-yl)-1-(3-(trifluoromethyl)phenyl)-1H-imidazo[4,5-c]quinolin-2(3H)-one

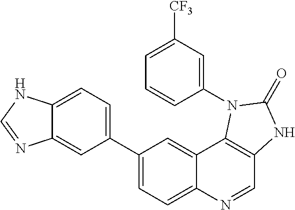 [SMILES: O=c1[nH]c2cnc3ccc(-c4ccc5[nH]cnc5c4)cc3c2n1-c1cccc(C(F)(F)F)c1]

¹H NMR (400 MHz, DMSO-d₆) δ 12.52 (s, 1H), 11.78 (s, 1H), 8.74 (s, 1H), 8.21 (d, J=13.4 Hz, 2H), 8.04 (dd, J=15.0, 8.3 Hz, 3H), 7.91 (s, 2H), 7.65-7.43 (m, 2H), 7.17 (d, J=2.0 Hz, 1H), 7.10 (dd, J=27.4, 8.6 Hz, 1H); LC/MS (Method B): (electrospray+ve), m/z 446.1 (MH)⁺, t_{R}=3.190, UV₂₅₄=100%

### 8-(4-(Methylsulfonyl)phenyl)-1-(3-(trifluoromethyl)phenyl)-1H-imidazo[4,5-c]quinolin-2(3H)-one

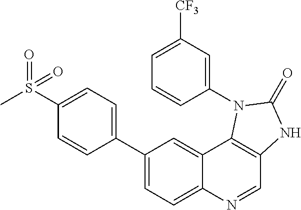 [SMILES: CS(=O)(=O)c1ccc(-c2ccc3ncc4[nH]c(=O)n(-c5cccc(C(F)(F)F)c5)c4c3c2)cc1]

¹H NMR (400 MHz, DMSO-d₆) δ 8.81 (s, 1H), 8.22 (d, J=2.0 Hz, 1H), 8.13 (d, J=8.8 Hz, 1H), 8.08-8.05 (m, 1H), 8.05-8.01 (m, 1H), 7.96-7.93 (m, 1H), 7.92 (d, J=2.1 Hz, 1H), 7.92-7.89 (m, 2H), 7.59-7.55 (m, 2H), 7.22 (d, J=2.0 Hz, 1H), 3.23 (s, 3H), 2.97 (d, J=5.2 Hz, 1H); LC/MS (Method B): (electrospray+ve), m/z 484.1 (MH)⁺, t_{R}=3.976, UV₂₅₄=100%

### 3-(2-Oxo-1-(3-(trifluoromethyl)phenyl)-2,3-dihydro-1H-imidazo[4,5-c]quinolin-8-yl)benzonitrile

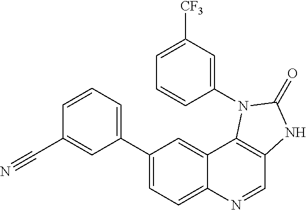 [SMILES: N#Cc1cccc(-c2ccc3ncc4[nH]c(=O)n(-c5cccc(C(F)(F)F)c5)c4c3c2)c1]

¹H NMR (400 MHz, DMSO-d₆) δ 11.85 (s, 1H), 8.80 (s, 1H), 8.23 (s, 1H), 8.11 (d, J=8.8 Hz, 1H), 8.03 (dd, J=8.6, 1.5 Hz, 3H), 7.80 (dt, J=7.6, 1.4 Hz, 1H), 7.69 (t, J=1.7 Hz, 2H), 7.66 (dt, J=8.0, 1.5 Hz, 1H), 7.59 (d, J=7.7 Hz, 1H), 7.12 (d, J=1.9 Hz, 1H); LC/MS (Method B): (electrospray+ve), m/z 431.1 (MH)⁺, t_{R}=4.417, UV₂₅₄=100%

### 8-(Quinolin-3-yl)-1-(3-(trifluoromethyl)phenyl)-1H-imidazo[4,5-c]quinolin-2(3H)-one

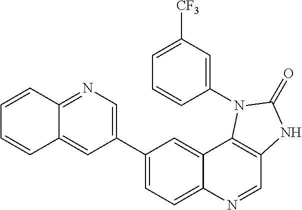 [SMILES: O=c1[nH]c2cnc3ccc(-c4cnc5ccccc5c4)cc3c2n1-c1cccc(C(F)(F)F)c1]

¹H NMR (400 MHz, DMSO-d₆) δ 11.85 (s, 1H), 8.82 (d, J=2.4 Hz, 1H), 8.80 (s, 1H), 8.33 (d, J=2.5 Hz, 1H), 8.22 (s, 1H), 8.16 (d, J=8.9 Hz, 1H), 8.10-8.02 (m, 4H), 7.96 (d, J=8.0 Hz, 1H), 7.95-7.91 (m, 1H), 7.77 (ddd, J=8.4, 6.9, 1.5 Hz, 1H), 7.66 (ddd, J=8.1, 6.9, 1.3 Hz, 1H), 7.32 (d, J=2.0 Hz, 1H); LC/MS (Method B): (electrospray+ve), m/z 457.1 (MH)⁺, t_{R}=4.177, UV₂₅₄=100%

### 8-(2-Aminopyrimidin-5-yl)-1-(3-(trifluoromethyl)phenyl)-1H-imidazo[4,5-c]quinolin-2(3H)-one

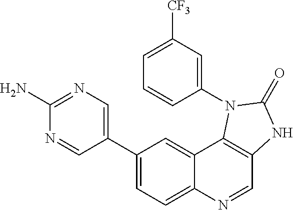 [SMILES: Nc1ncc(-c2ccc3ncc4[nH]c(=O)n(-c5cccc(C(F)(F)F)c5)c4c3c2)cn1]

¹H NMR (400 MHz, DMSO-d₆) δ 11.78 (s, 1H), 8.73 (s, 1H), 8.20 (s, 2H), 8.16 (s, 1H), 8.03 (d, J=8.9 Hz, 2H), 8.02-7.98 (m, 1H), 7.93 (d, J=7.9 Hz, 1H), 7.83 (dd, J=8.9, 2.1 Hz, 1H), 6.97 (d, J=2.0 Hz, 1H), 6.87 (s, 2H).; LC/MS (Method B): (electrospray+ve), m/z 423.1 (MH)⁺, t_{R}=3.377, UV₂₅₄=100%

### 4-(2-Oxo-1-(3-(trifluoromethyl)phenyl)-2,3-dihydro-1H-imidazo[4,5-c]quinolin-8-yl)benzamide

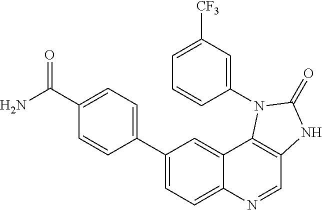 [SMILES: NC(=O)c1ccc(-c2ccc3ncc4[nH]c(=O)n(-c5cccc(C(F)(F)F)c5)c4c3c2)cc1]

¹H NMR (400 MHz, DMSO-d₆) δ 11.86 (s, 1H), 8.80 (s, 1H), 8.22 (s, 1H), 8.10 (d, J=8.9 Hz, 1H), 8.06-8.00 (m, 3H), 7.93 (d, J=8.2 Hz, 2H), 7.86 (d, J=8.4 Hz, 2H), 7.39 (s, 2H), 7.37 (s, 1H), 7.18 (d, J=2.1 Hz, 1H); LC/MS (Method B): (electrospray+ve), m/z 449.1 (MH)⁺, t_{R}=3.540, UV₂₅₄=100%

### 8-(2-Aminopyrimidin-5-yl)-1-(3-(trifluoromethyl)phenyl)-1H-imidazo[4,5-c]quinolin-2(3H)-one

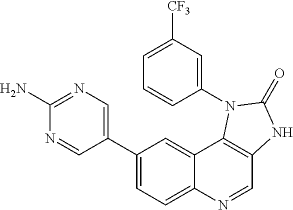 [SMILES: Nc1ncc(-c2ccc3ncc4[nH]c(=O)n(-c5cccc(C(F)(F)F)c5)c4c3c2)cn1]

¹H NMR (400 MHz, DMSO-d₆) δ 12.09 (s, 1H), 8.88 (s, 1H), 8.19 (s, 3H), 8.10 (d, J=9.0 Hz, 1H), 8.07 (d, J=7.0 Hz, 1H), 8.05-8.01 (m, 1H), 7.98-7.92 (m, 2H), 7.01-6.90 (m, 3H); LC/MS (Method B): (electrospray+ve), m/z 423.1 (MH)⁺, t_{R}=3.387, UV₂₅₄=100%

### 8-(4-Aminophenyl)-3-methyl-1-(3-(trifluoromethyl)phenyl)-1H-imidazo[4,5-c]quinolin-2(3H)-one

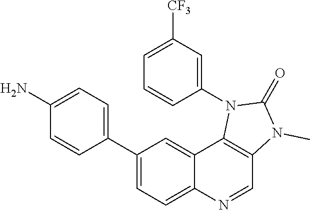 [SMILES: Cn1c(=O)n(-c2cccc(C(F)(F)F)c2)c2c3cc(-c4ccc(N)cc4)ccc3ncc21]

¹H NMR (400 MHz, DMSO-d₆) δ 9.14 (s, 1H), 8.24 (s, 1H), 8.11 (d, J=7.3 Hz, 1H), 8.08 (d, J=9.1 Hz, 1H), 8.04 (d, J=8.0 Hz, 1H), 7.98 (d, J=8.0 Hz, 2H), 7.03 (d, J=8.6 Hz, 2H), 7.00 (d, J=2.0 Hz, 1H), 6.59 (d, J=8.3 Hz, 2H), 3.61 (s, 4H), 2.51 (s, 1H); LC/MS (Method B): (electrospray+ve), m/z 435.1 (MH)⁺, t_{R}=3.774, UV₂₅₄=100%

### 4-(3-Methyl-2-oxo-1-(3-(trifluoromethyl)phenyl)-2,3-dihydro-1H-imidazo[4,5-c]quinolin-8-yl)benzamide

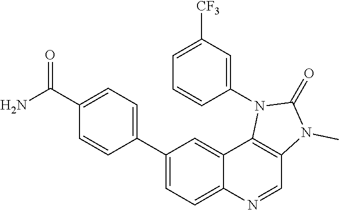 [SMILES: Cn1c(=O)n(-c2cccc(C(F)(F)F)c2)c2c3cc(-c4ccc(C(N)=O)cc4)ccc3ncc21]

¹H NMR (400 MHz, DMSO-d₆) δ 9.12 (s, 1H), 8.23 (s, 1H), 8.16 (d, J=8.9 Hz, 1H), 8.09 (d, J=7.9 Hz, 1H), 8.03 (t, J=9.0 Hz, 3H), 7.96 (d, J=7.9 Hz, 1H), 7.86 (d, J=8.3 Hz, 2H), 7.38 (d, J=8.3 Hz, 3H), 7.19 (s, 1H), 3.62 (s, 3H); LC/MS (Method B): (electrospray+ve), m/z 463.1 (MH)⁺, t_{R}=3.741, UV₂₅₄=100%

### N-(5-(3-Methyl-2-oxo-1-(3-(trifluoromethyl)phenyl)-2,3-dihydro-1H-imidazo[4,5-c]quinolin-8-yl)pyridin-2-yl)acetamide

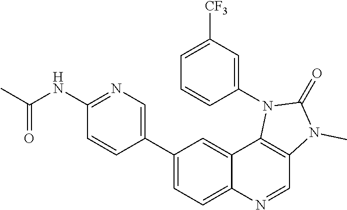 [SMILES: CC(=O)Nc1ccc(-c2ccc3ncc4c(c3c2)n(-c2cccc(C(F)(F)F)c2)c(=O)n4C)cn1]

¹H NMR (400 MHz, DMSO-d₆) δ 10.64 (s, 1H), 9.10 (s, 1H), 8.21 (s, 1H), 8.18 (d, J=2.5 Hz, 1H), 8.15 (d, J=8.9 Hz, 1H), 8.08 (dd, J=8.6, 2.4 Hz, 2H), 8.04 (d, J=7.7 Hz, 1H), 7.96 (d, J=7.9 Hz, 1H), 7.77 (dd, J=8.7, 2.6 Hz, 1H), 7.12 (d, J=2.1 Hz, 1H), 3.61 (s, 4H), 2.08 (s, 3H); LC/MS (Method B): (electrospray+ve), m/z 478.1 (MH)⁺, t_{R}=3.894, UV₂₅₄=100%

### 8-(3-Aminophenyl)-3-methyl-1-(3-(trifluoromethyl)phenyl)-1H-imidazo[4,5-c]quinolin-2(3H)-one

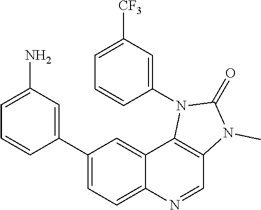 [SMILES: Cn1c(=O)n(-c2cccc(C(F)(F)F)c2)c2c3cc(-c4cccc(N)c4)ccc3ncc21]

¹H NMR (400 MHz, DMSO-d₆) δ 9.15 (s, 1H), 8.19 (s, 1H), 8.14 (d, J=8.9 Hz, 1H), 8.07 (d, J=7.7 Hz, 1H), 8.04 (d, J=8.1 Hz, 1H), 7.98 (t, J=7.8 Hz, 1H), 7.88 (d, J=8.8 Hz, 1H), 7.12 (d, J=2.0 Hz, 1H), 7.04 (t, J=7.8 Hz, 1H), 6.73 (s, 1H), 6.65 (d, J=7.9 Hz, 1H), 6.45 (d, J=7.6 Hz, 1H), 3.62 (s, 4H); LC/MS (Method B): (electrospray+ve), m/z 435.1 (MH)⁺, t_{R}=3.775, UV₂₅₄=100%

### 8-(2-Aminopyrimidin-5-yl)-3-methyl-1-(3-(trifluoromethyl)phenyl)-1H-imidazo[4,5-c]quinolin-2(3H)-one

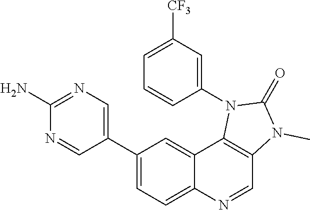 [SMILES: Cn1c(=O)n(-c2cccc(C(F)(F)F)c2)c2c3cc(-c4cnc(N)nc4)ccc3ncc21]

¹H NMR (400 MHz, DMSO-d₆) δ 9.13 (s, 1H), 8.20 (s, 2H), 8.19 (s, 1H), 8.12 (d, J=8.9 Hz, 1H), 8.09 (d, J=7.8 Hz, 1H), 8.04 (d, J=8.2 Hz, 1H), 8.00-7.97 (m, 1H), 7.96 (d, J=4.2 Hz, 1H), 7.04-6.90 (m, 3H), 3.61 (s, 4H); LC/MS (Method B): (electrospray+ve), m/z 437.1 (MH)⁺, t_{R}=3.531, UV₂₅₄=100%

### 3-(3-Methyl-2-oxo-1-(3-(trifluoromethyl)phenyl)-2,3-dihydro-1H-imidazo[4,5-c]quinolin-8-yl)benzonitrile

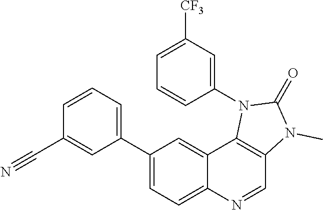 [SMILES: Cn1c(=O)n(-c2cccc(C(F)(F)F)c2)c2c3cc(-c4cccc(C#N)c4)ccc3ncc21]

¹H NMR (400 MHz, DMSO-d₆) δ 9.11 (s, 1H), 8.22 (s, 1H), 8.16 (d, J=8.9 Hz, 1H), 8.05 (d, J=7.6 Hz, 2H), 8.00 (dd, J=8.8, 2.0 Hz, 1H), 7.95 (t, J=7.9 Hz, 1H), 7.81 (d, J=7.4 Hz, 1H), 7.69 (d, J=1.9 Hz, 1H), 7.65 (d, J=8.1 Hz, 1H), 7.58 (t, J=7.8 Hz, 1H), 7.13 (d, J=2.0 Hz, 1H), 3.61 (s, 4H); LC/MS (Method B): (electrospray+ve), m/z 445.1 (MH)⁺, t_{R}=4.582, UV₂₅₄=100%

### Example 12

This example demonstrates the gametocytocidal activity and activity against asexual parasites in accordance with an embodiment of the invention.

Compounds were screened against gametocytes and asexual parasites as described in Example 3. The results are set forth in Tables 6-8.

### Table 6

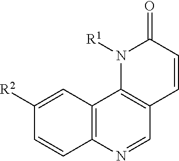

| | | | Activity |
|---|---|---|---|
| | | Gametocytocidal | against asexual |
| | | activity EC₅₀ | parasites EC₅₀ |
| R¹ | R² | (nM) | (nM) |
| 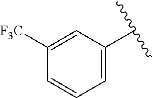 [SMILES: *c1cccc(C(F)(F)F)c1] | 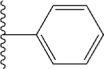 [SMILES: *c1ccccc1] | 627 | 113 |
| 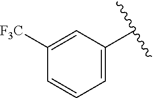 [SMILES: *c1cccc(C(F)(F)F)c1] | 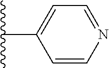 [SMILES: *c1ccncc1] | 246 | 92 |
| 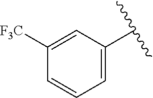 [SMILES: *c1cccc(C(F)(F)F)c1] | 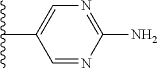 [SMILES: *c1cnc(N)nc1] | 40 | 22 |
| 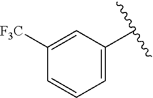 [SMILES: *c1cccc(C(F)(F)F)c1] | 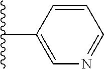 [SMILES: *c1cccnc1] | 535 | 110 |
| 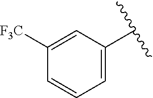 [SMILES: *c1cccc(C(F)(F)F)c1] | 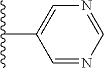 [SMILES: *c1cncnc1] | 942 | 475 |
| Ethyl | 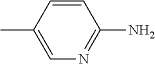 | 1650 | 1274 |
| 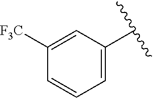 [SMILES: *c1cccc(C(F)(F)F)c1] | 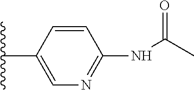 [SMILES: *c1ccc(NC(C)=O)nc1] | 114 | 106 |
| 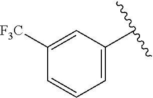 [SMILES: *c1cccc(C(F)(F)F)c1] | 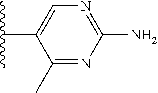 [SMILES: *c1cnc(N)nc1C] | 2263 | 1150 |
| 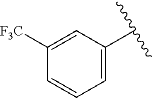 [SMILES: *c1cccc(C(F)(F)F)c1] | 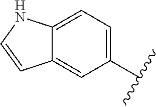 [SMILES: *c1ccc2[nH]ccc2c1] | 325 | 191 |
| 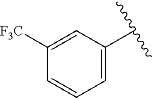 [SMILES: *c1cccc(C(F)(F)F)c1] | 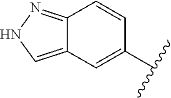 [SMILES: *c1ccc2n[nH]cc2c1] | 74 | 64 |
| 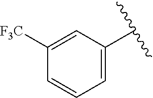 [SMILES: *c1cccc(C(F)(F)F)c1] | 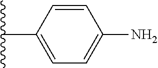 [SMILES: *c1ccc(N)cc1] | 147 | 99 |
| 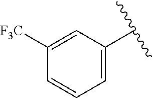 [SMILES: *c1cccc(C(F)(F)F)c1] | 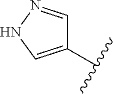 [SMILES: *c1cn[nH]c1] | 246 | 131 |
| 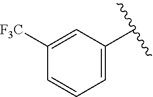 [SMILES: *c1cccc(C(F)(F)F)c1] | 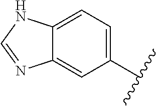 [SMILES: *c1ccc2[nH]cnc2c1] | 96 | 37 |
| 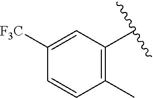 [SMILES: *c1cc(C(F)(F)F)ccc1C] | 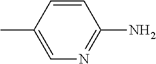 | 65 | 44 |
| 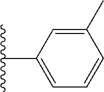 [SMILES: *c1cccc(C)c1] | 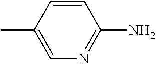 | 19 | 23 |
| 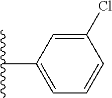 [SMILES: *c1cccc(Cl)c1] | 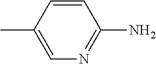 | 18 | 10 |
| 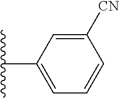 [SMILES: *c1cccc(C#N)c1] | 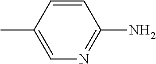 | 23 | 12 |
| 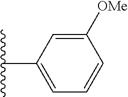 [SMILES: *c1cccc(OC)c1] | 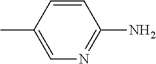 | 137 | 95 |
| 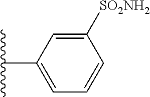 [SMILES: *c1cccc(S(N)(=O)=O)c1] | 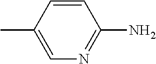 | 295 | 82 |
| 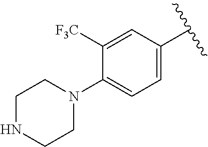 [SMILES: *c1ccc(N2CCNCC2)c(C(F)(F)F)c1] | 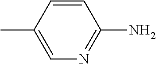 | 10 | 9 |
| 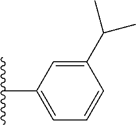 [SMILES: *c1cccc(C(C)C)c1] | 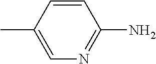 | ND | ND |
| 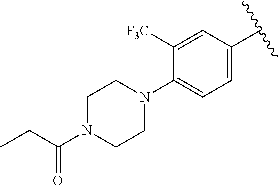 [SMILES: *c1ccc(N2CCN(C(=O)CC)CC2)c(C(F)(F)F)c1] | 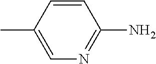 | ND | ND |
| 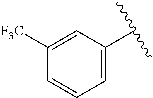 [SMILES: *c1cccc(C(F)(F)F)c1] | 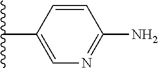 [SMILES: *c1ccc(N)nc1] | 18 | 6 |
| 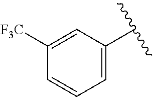 [SMILES: *c1cccc(C(F)(F)F)c1] | 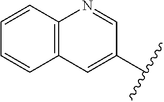 [SMILES: *c1cnc2ccccc2c1] | 2270 | 1183 |
| 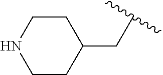 [SMILES: *CC1CCNCC1] | 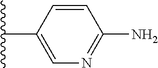 [SMILES: *c1ccc(N)nc1] | 11520 | 6436 |
| 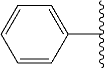 [SMILES: *c1ccccc1] | 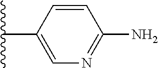 [SMILES: *c1ccc(N)nc1] | 47 | 33 |
| 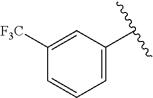 [SMILES: *c1cccc(C(F)(F)F)c1] | 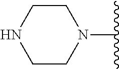 [SMILES: *N1CCNCC1] | 22040 | >20000 |
| 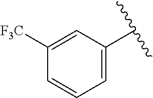 [SMILES: *c1cccc(C(F)(F)F)c1] | 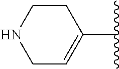 [SMILES: *C1=CCNCC1] | 18390 | 15720 |
| 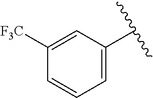 [SMILES: *c1cccc(C(F)(F)F)c1] | 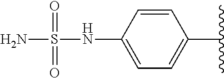 [SMILES: *c1ccc(NS(N)(=O)=O)cc1] | 2652 | 1512 |
| 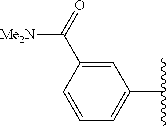 [SMILES: *c1cccc(C(=O)N(C)C)c1] | 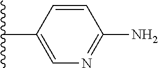 [SMILES: *c1ccc(N)nc1] | 1519 | 859 |
| 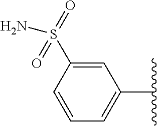 [SMILES: *c1cccc(S(N)(=O)=O)c1] | 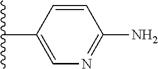 [SMILES: *c1ccc(N)nc1] | 295 | 82 |
| 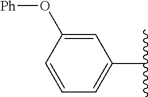 [SMILES: *c1cccc(Oc2ccccc2)c1] | 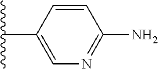 [SMILES: *c1ccc(N)nc1] | 2427 | 1667 |
| 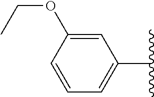 [SMILES: *c1cccc(OCC)c1] | 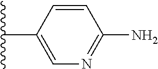 [SMILES: *c1ccc(N)nc1] | 912 | 683 |
| 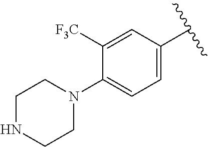 [SMILES: *c1ccc(N2CCNCC2)c(C(F)(F)F)c1] | 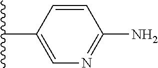 [SMILES: *c1ccc(N)nc1] | 10 | 9 |
| 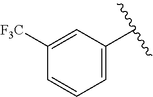 [SMILES: *c1cccc(C(F)(F)F)c1] | 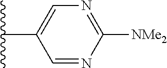 [SMILES: *c1cnc(N(C)C)nc1] | 3131 | 1951 |
| 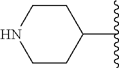 [SMILES: *C1CCNCC1] | 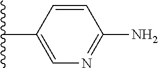 [SMILES: *c1ccc(N)nc1] | 2804 | 2374 |
| 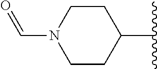 [SMILES: *C1CCN(C=O)CC1] | 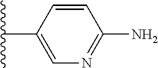 [SMILES: *c1ccc(N)nc1] | 950 | 880 |
| 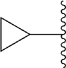 [SMILES: *C1CC1] | 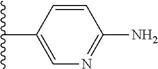 [SMILES: *c1ccc(N)nc1] | 215 | 560 |
| 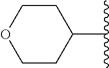 [SMILES: *C1CCOCC1] | 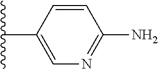 [SMILES: *c1ccc(N)nc1] | 41 | 1338 |
| 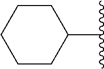 [SMILES: *C1CCCCC1] | 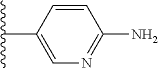 [SMILES: *c1ccc(N)nc1] | 676 | 707 |
| 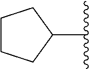 [SMILES: *C1CCCC1] | 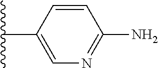 [SMILES: *c1ccc(N)nc1] | 1263 | 882 |
| 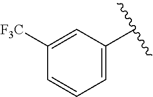 [SMILES: *c1cccc(C(F)(F)F)c1] | 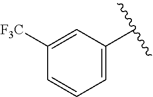 [SMILES: *c1cccc(C(F)(F)F)c1] | 3378 | 4198 |
| 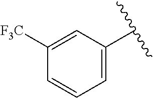 [SMILES: *c1cccc(C(F)(F)F)c1] | Br | 4950 | 5582 |
| 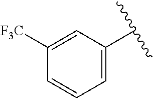 [SMILES: *c1cccc(C(F)(F)F)c1] | 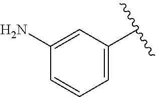 [SMILES: *c1cccc(N)c1] | 103 | 120 |
| 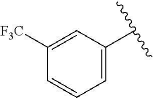 [SMILES: *c1cccc(C(F)(F)F)c1] | 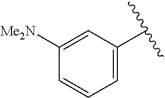 [SMILES: *c1cccc(N(C)C)c1] | 3293 | 3253 |
| 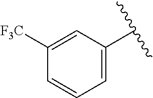 [SMILES: *c1cccc(C(F)(F)F)c1] | CH₂═CH | 4899 | 3240 |
| 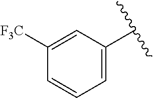 [SMILES: *c1cccc(C(F)(F)F)c1] | 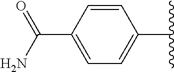 [SMILES: *c1ccc(C(N)=O)cc1] | 1273 | 1057 |
| 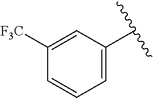 [SMILES: *c1cccc(C(F)(F)F)c1] | 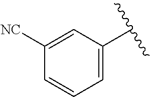 [SMILES: *c1cccc(C#N)c1] | 526 | 283 |
| 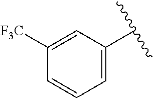 [SMILES: *c1cccc(C(F)(F)F)c1] | 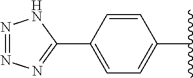 [SMILES: *c1ccc(-c2nnn[nH]2)cc1] | 20930 | >20000 |
| 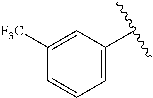 [SMILES: *c1cccc(C(F)(F)F)c1] | 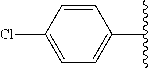 [SMILES: *c1ccc(Cl)cc1] | 673 | 253 |
| 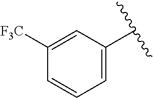 [SMILES: *c1cccc(C(F)(F)F)c1] | 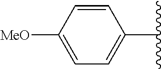 [SMILES: *c1ccc(OC)cc1] | 3240 | 2457 |
| 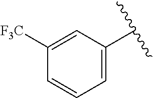 [SMILES: *c1cccc(C(F)(F)F)c1] | 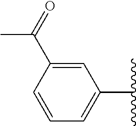 [SMILES: *c1cccc(C(C)=O)c1] | 1239 | 352 |
| 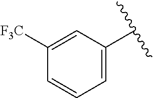 [SMILES: *c1cccc(C(F)(F)F)c1] | 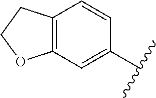 [SMILES: *c1ccc2c(c1)OCC2] | 3311 | 2239 |
| 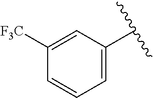 [SMILES: *c1cccc(C(F)(F)F)c1] | 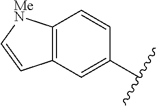 [SMILES: *c1ccc2c(ccn2C)c1] | 3256 | 2088 |
| 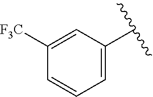 [SMILES: *c1cccc(C(F)(F)F)c1] | 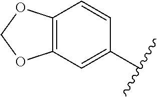 [SMILES: *c1ccc2c(c1)OCO2] | 3129 | 802 |
| 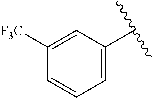 [SMILES: *c1cccc(C(F)(F)F)c1] | 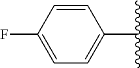 [SMILES: *c1ccc(F)cc1] | 2660 | 580 |
| 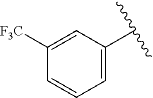 [SMILES: *c1cccc(C(F)(F)F)c1] | 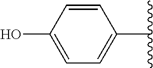 [SMILES: *c1ccc(O)cc1] | 165 | 129 |
| 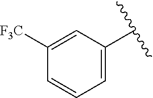 [SMILES: *c1cccc(C(F)(F)F)c1] | 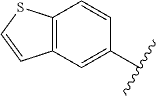 [SMILES: *c1ccc2sccc2c1] | 3213 | 1639 |
| 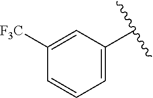 [SMILES: *c1cccc(C(F)(F)F)c1] | 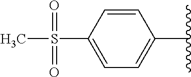 [SMILES: *c1ccc(S(C)(=O)=O)cc1] | 491 | 266 |
| 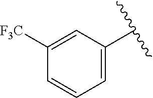 [SMILES: *c1cccc(C(F)(F)F)c1] | 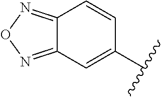 [SMILES: *c1ccc2nonc2c1] | 4523 | 1725 |
| 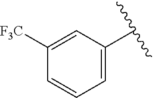 [SMILES: *c1cccc(C(F)(F)F)c1] | 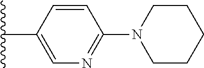 [SMILES: *c1ccc(N2CCCCC2)nc1] | 8093 | 4905 |
| 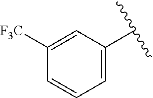 [SMILES: *c1cccc(C(F)(F)F)c1] | 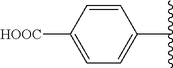 [SMILES: *c1ccc(C(=O)O)cc1] | 3029 | 6304 |
| 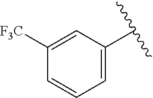 [SMILES: *c1cccc(C(F)(F)F)c1] | 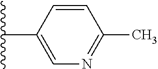 [SMILES: *c1ccc(C)nc1] | 65 | 33 |
| 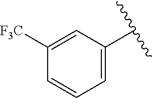 [SMILES: *c1cccc(C(F)(F)F)c1] | 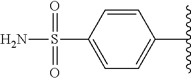 [SMILES: *c1ccc(S(N)(=O)=O)cc1] | 303 | 384 |
| 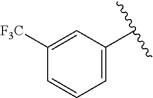 [SMILES: *c1cccc(C(F)(F)F)c1] | 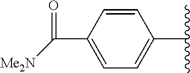 [SMILES: *c1ccc(C(=O)N(C)C)cc1] | 1989 | 2085 |
| 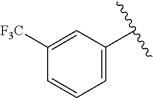 [SMILES: *c1cccc(C(F)(F)F)c1] | 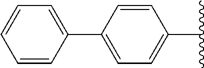 [SMILES: *c1ccc(-c2ccccc2)cc1] | 19300 | 14950 |
| 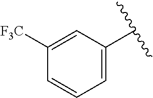 [SMILES: *c1cccc(C(F)(F)F)c1] | 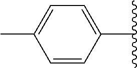 [SMILES: *c1ccc(C)cc1] | 794 | 372 |
| 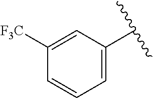 [SMILES: *c1cccc(C(F)(F)F)c1] | 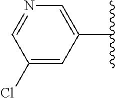 [SMILES: *c1cncc(Cl)c1] | 8795 | 3593 |
| 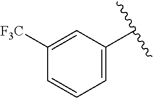 [SMILES: *c1cccc(C(F)(F)F)c1] | 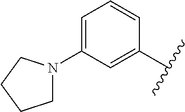 [SMILES: *c1cccc(N2CCCC2)c1] | 16120 | 13320 |
| 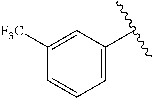 [SMILES: *c1cccc(C(F)(F)F)c1] | 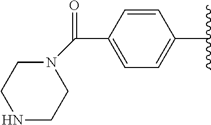 [SMILES: *c1ccc(C(=O)N2CCNCC2)cc1] | 5571 | 7873 |
| 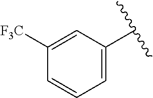 [SMILES: *c1cccc(C(F)(F)F)c1] | 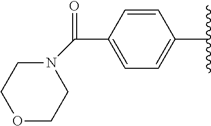 [SMILES: *c1ccc(C(=O)N2CCOCC2)cc1] | 22420 | 12050 |
| 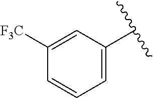 [SMILES: *c1cccc(C(F)(F)F)c1] | 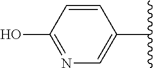 [SMILES: *c1ccc(O)nc1] | 262 | 187 |
| 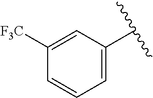 [SMILES: *c1cccc(C(F)(F)F)c1] | 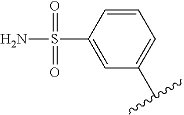 [SMILES: *c1cccc(S(N)(=O)=O)c1] | 342 | 128 |
| 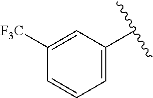 [SMILES: *c1cccc(C(F)(F)F)c1] | 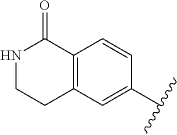 [SMILES: *c1ccc2c(c1)CCNC2=O] | 3170 | 1134 |
| 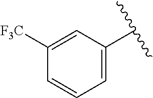 [SMILES: *c1cccc(C(F)(F)F)c1] | 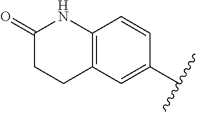 [SMILES: *c1ccc2c(c1)CCC(=O)N2] | 3170 | 1134 |
| 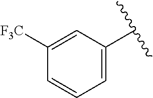 [SMILES: *c1cccc(C(F)(F)F)c1] | CH₂NH₂ | 281 | 163 |
| 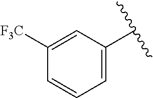 [SMILES: *c1cccc(C(F)(F)F)c1] | CH₂NMe₂ | 666 | 242 |
| 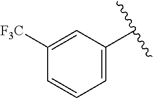 [SMILES: *c1cccc(C(F)(F)F)c1] | 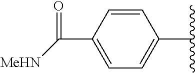 [SMILES: *c1ccc(C(=O)NC)cc1] | 61 | 17 |
| 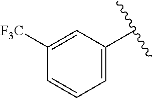 [SMILES: *c1cccc(C(F)(F)F)c1] | 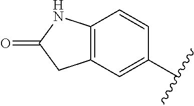 [SMILES: *c1ccc2c(c1)CC(=O)N2] | 51 | 19 |
| 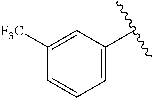 [SMILES: *c1cccc(C(F)(F)F)c1] | 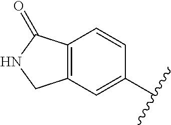 [SMILES: *c1ccc2c(c1)CNC2=O] | 1793 | 517 |

TABLE 7

| | Gametocytocidal | Activity against asexual |
|---|---|---|
| Compound | activity EC₅₀ (nM) | parasites EC₅₀ (nM) |
| 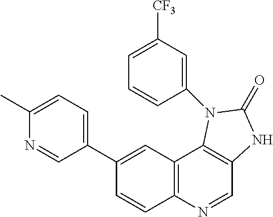 [SMILES: Cc1ccc(-c2ccc3ncc4[nH]c(=O)n(-c5cccc(C(F)(F)F)c5)c4c3c2)cn1] | 443 | 59 |
| 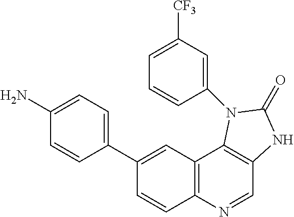 [SMILES: Nc1ccc(-c2ccc3ncc4[nH]c(=O)n(-c5cccc(C(F)(F)F)c5)c4c3c2)cc1] | 630 | 153 |
| 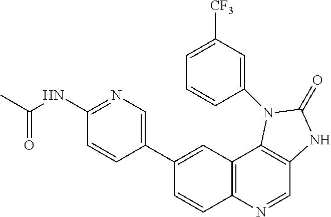 [SMILES: CC(=O)Nc1ccc(-c2ccc3ncc4[nH]c(=O)n(-c5cccc(C(F)(F)F)c5)c4c3c2)cn1] | 123 | 35 |
| 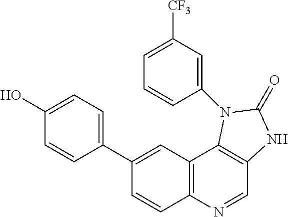 [SMILES: O=c1[nH]c2cnc3ccc(-c4ccc(O)cc4)cc3c2n1-c1cccc(C(F)(F)F)c1] | 1421 | 398 |
| 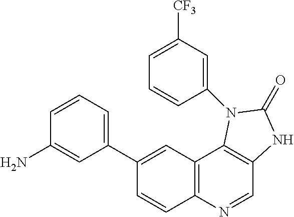 [SMILES: Nc1cccc(-c2ccc3ncc4[nH]c(=O)n(-c5cccc(C(F)(F)F)c5)c4c3c2)c1] | 531 | 137 |
| 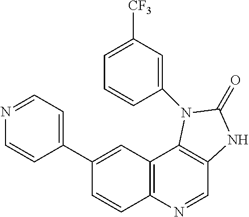 [SMILES: O=c1[nH]c2cnc3ccc(-c4ccncc4)cc3c2n1-c1cccc(C(F)(F)F)c1] | 1237 | 211 |
| 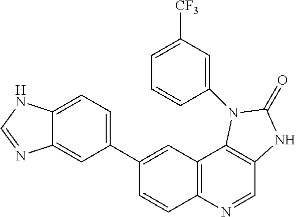 [SMILES: O=c1[nH]c2cnc3ccc(-c4ccc5[nH]cnc5c4)cc3c2n1-c1cccc(C(F)(F)F)c1] | 543 | 90 |
| 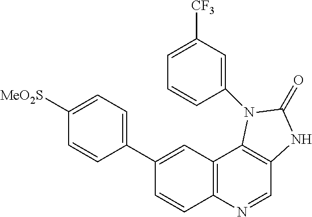 [SMILES: CS(=O)(=O)c1ccc(-c2ccc3ncc4[nH]c(=O)n(-c5cccc(C(F)(F)F)c5)c4c3c2)cc1] | 2046 | 622 |
| 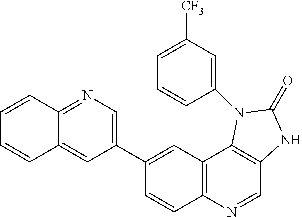 [SMILES: O=c1[nH]c2cnc3ccc(-c4cnc5ccccc5c4)cc3c2n1-c1cccc(C(F)(F)F)c1] | 212000 | 5785 |
| 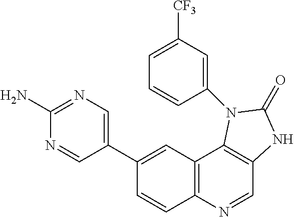 [SMILES: Nc1ncc(-c2ccc3ncc4[nH]c(=O)n(-c5cccc(C(F)(F)F)c5)c4c3c2)cn1] | 246 | 57 |
| 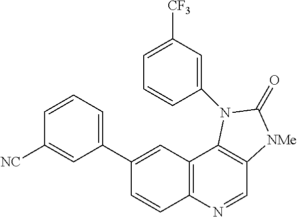 [SMILES: Cn1c(=O)n(-c2cccc(C(F)(F)F)c2)c2c3cc(-c4cccc(C#N)c4)ccc3ncc21] | 2818 | 546 |
| 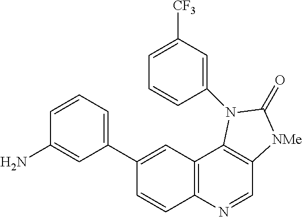 [SMILES: Cn1c(=O)n(-c2cccc(C(F)(F)F)c2)c2c3cc(-c4cccc(N)c4)ccc3ncc21] | 69 | 16 |
| 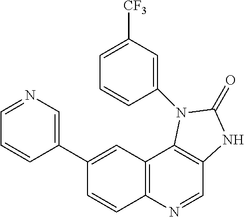 [SMILES: O=c1[nH]c2cnc3ccc(-c4cccnc4)cc3c2n1-c1cccc(C(F)(F)F)c1] | 5574 | 1398 |
| 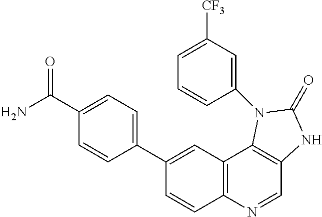 [SMILES: NC(=O)c1ccc(-c2ccc3ncc4[nH]c(=O)n(-c5cccc(C(F)(F)F)c5)c4c3c2)cc1] | 227 | 45 |
| 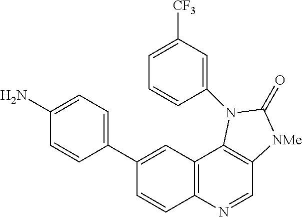 [SMILES: Cn1c(=O)n(-c2cccc(C(F)(F)F)c2)c2c3cc(-c4ccc(N)cc4)ccc3ncc21] | 69 | 16 |
| 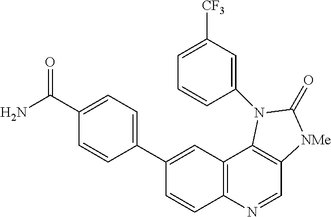 [SMILES: Cn1c(=O)n(-c2cccc(C(F)(F)F)c2)c2c3cc(-c4ccc(C(N)=O)cc4)ccc3ncc21] | 109 | 20 |
| 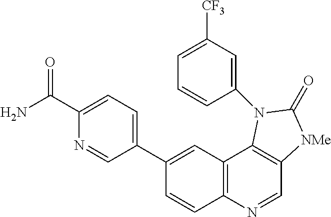 [SMILES: Cn1c(=O)n(-c2cccc(C(F)(F)F)c2)c2c3cc(-c4ccc(C(N)=O)nc4)ccc3ncc21] | 43 | 8 |
| 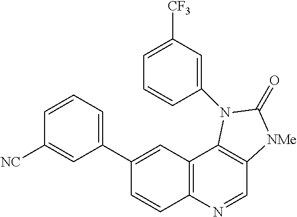 [SMILES: Cn1c(=O)n(-c2cccc(C(F)(F)F)c2)c2c3cc(-c4cccc(C#N)c4)ccc3ncc21] | 314 | 59 |
| 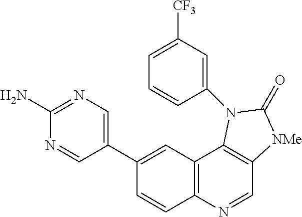 [SMILES: Cn1c(=O)n(-c2cccc(C(F)(F)F)c2)c2c3cc(-c4cnc(N)nc4)ccc3ncc21] | 31 | 7 |
| 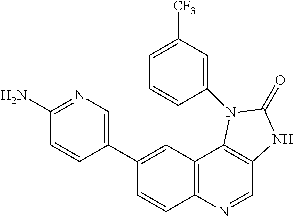 [SMILES: Nc1ccc(-c2ccc3ncc4[nH]c(=O)n(-c5cccc(C(F)(F)F)c5)c4c3c2)cn1] | 88 | 105 |

TABLE 8

| | Gametocytocidal | Activity against asexual |
|---|---|---|
| Compound | activity EC₅₀ (nM) | parasites EC₅₀ (nM) |
| 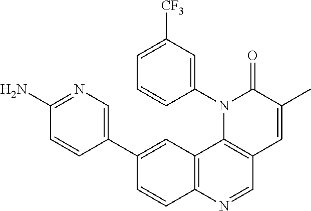 [SMILES: Cc1cc2cnc3ccc(-c4ccc(N)nc4)cc3c2n(-c2cccc(C(F)(F)F)c2)c1=O] | 46 | 205 |
| 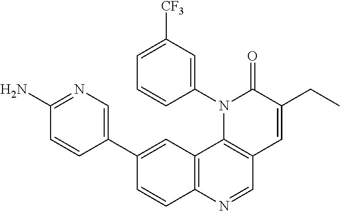 [SMILES: CCc1cc2cnc3ccc(-c4ccc(N)nc4)cc3c2n(-c2cccc(C(F)(F)F)c2)c1=O] | 136 | 260 |

### REFERENCES

1. Kilama W, Ntoumi F (2009) Malaria: a research agenda for the eradication era. Lancet 374: 1480-1482.
2. Buchholz K, Burke T A, Williamson K C, Wiegand R C, Wirth D F, et al. (2011) A high-throughput screen targeting malaria transmission stages opens new avenues for drug development. The Journal of infectious diseases 203: 1445-1453.
3. Sweeney A W, Blackburn C R B, Rieckmann K H (2004) Short report: The activity of pamaquine, an 8-aminoquinoline drug, against sporozoite-induced infections of Plasmodium vivax (New Guinea strains). American Journal of Tropical Medicine and Hygiene 71: 187-189.
4. Peatey C L, Skinner-Adams T S, Dixon M W A, McCarthy J S, Gardiner D L, et al. (2009) Effect of Antimalarial Drugs on Plasmodium falciparum Gametocytes. Journal of Infectious Diseases 200: 1518-1521.
5. Baird J K, Surjadjaja C (2011) Consideration of ethics in primaquine therapy against malaria transmission. Trends in Parasitology 27: 11-16.
6. Song J P, Socheat D, Tan B, Dara P, Deng C S, et al. (2010) Rapid and effective malaria control in Cambodia through mass administration of artemisinin-piperaquine. Malaria Journal 9.
7. Shekalaghe S A, Drakeley C, van den Bosch S, ter Braak R, van den Bijllaardt W, et al. (2011) A cluster-randomized trial of mass drug administration with a gametocytocidal drug combination to interrupt malaria transmission in a low endemic area in Tanzania. Malaria Journal 10.
8. Coulibaly B, Zoungrana A, Mockenhaupt F P, Schirmer R H, Klose C, et al. (2009) Strong gametocytocidal effect of methylene blue-based combination therapy against falciparum malaria: a randomised controlled trial. PloS one 4: e5318.
9. Peatey C L, Spicer T P, Hodder P S, Trenholme K R, Gardiner D L (2011) A high-throughput assay for the identification of drugs against late-stage Plasmodium falciparum gametocytes. Molecular and biochemical parasitology 180: 127-131.
10. Tanaka T Q, Williamson K C (2011) A malaria gametocytocidal assay using oxidoreduction indicator, alamarBlue. Molecular and biochemical parasitology 177: 160-163.
11. Tanaka T Q, Dehdashti S J, Nguyen D T, McKew J C, Zheng W, et al. (2013) A quantitative high throughput assay for identifying gametocytocidal compounds. Molecular and biochemical parasitology 188: 20-25.
12. Huang R, Southall N, Wang Y, Yasgar A, Shinn P, et al. (2011) The NCGC pharmaceutical collection: a comprehensive resource of clinically approved drugs enabling repurposing and chemical genomics. Science translational medicine 3: 80ps16.
13. Duffy S, Avery V M (2012) Development and Optimization of a Novel 384-Well Anti-Malarial Imaging Assay Validated for High-Throughput Screening. American Journal of Tropical Medicine and Hygiene 86: 84-92.
14. Mathews L A, Keller J M, Goodwin B L, Guha R, Shinn P, et al. (2012) A 1536-Well Quantitative High-Throughput Screen to Identify Compounds Targeting Cancer Stem Cells. Journal of Biomolecular Screening 17: 1231-1242.
15. Yuan J, Cheng K C, Johnson R L, Huang R, Pattaradilokrat S, et al. (2011) Chemical genomic profiling for antimalarial therapies, response signatures, and molecular targets. Science 333: 724-729.
16. Yuan J, Johnson R L, Huang R, Wichterman J, Jiang H, et al. (2009) Genetic mapping of targets mediating differential chemical phenotypes in Plasmodium falciparum. Nat Chem Biol 5: 765-771.
17. Kohonen T (2006) Self-organizing neural projections. Neural networks: the official journal of the International Neural Network Society 19: 723-733.
18. Tin F, Nyunt H (1984) Comparative drug trial of a sulfadoxine/pyrimethamine and a sulfalene/pyrimethamine combination against Plasmodium falciparum infections in semi-immune populations of Burma. The Southeast Asian journal of tropical medicine and public health 15: 238-248.
19. Butcher G A (1997) Antimalarial drugs and the mosquito transmission of Plasmodium. International journal for parasitology 27: 975-987.
20. Reininger L, Wilkes J M, Bourgade H, Miranda-Saavedra D, Doerig C (2011) An essential Aurora-related kinase transiently associates with spindle pole bodies during Plasmodium falciparum erythrocytic schizogony. Molecular microbiology 79: 205-221.
21. Andrews K T, Gupta A P, Tran T N, Fairlie D P, Gobert G N, et al. (2012) Comparative gene expression profiling of P. falciparum malaria parasites exposed to three different histone deacetylase inhibitors. PloS one 7: e31847.
22. Lucantoni L, Avery V (2012) Whole-cell in vitro screening for gametocytocidal compounds. Future medicinal chemistry 4: 2337-2360.
23. Chen P Q, Li G Q, Guo X B, He K R, Fu Y X, et al. (1994) The infectivity of gametocytes of Plasmodium falciparum from patients treated with artemisinin. Chinese medical journal 107: 709-711.
24. Bousema J T, Schneider P, Gouagna L C, Drakeley C J, Tostmann A, et al. (2006) Moderate effect of artemisinin-based combination therapy on transmission of Plasmodium falciparum. The Journal of infectious diseases 193: 1151-1159.
25. Fidock D A, Rosenthal P J, Croft S L, Brun R, Nwaka S (2004) Antimalarial drug discovery: efficacy models for compound screening. Nature reviews Drug discovery 3: 509-520.
26. Kidgell C, Volkman S K, Daily J, Borevitz J O, Plouffe D, et al. (2006) A systematic map of genetic variation in Plasmodium falciparum. PLoS pathogens 2: e57.
27. Baniecki M L, Wirth D F, Clardy J (2007) High-throughput Plasmodium falciparum growth assay for malaria drug discovery. Antimicrobial Agents and Chemotherapy 51: 716-723.
28. Lai C J, Bao R, Tao X, Wang J, Atoyan R, et al. (2010) CUDC-101, a multitargeted inhibitor of histone deacetylase, epidermal growth factor receptor, and human epidermal growth factor receptor 2, exerts potent anticancer activity. Cancer Research 70: 3647-3656.
29. Liu Q, Wang J, Kang S A, Thoreen C C, Hur W, et al. (2011) Discovery of 9-(6-aminopyridin-3-yl)-1-(3-(trifluoromethyl)phenyl)benzo[h][1,6]naphthyridin-2(1H)-one (Torin2) as a potent, selective, and orally available mammalian target of rapamycin (mTOR) inhibitor for treatment of cancer. Journal of Medicinal Chemistry 54: 1473-1480.
30. Liu Q, Xu C, Kirubakaran S, Zhang X, Hur W, et al. (2013) Characterization of Torin2, an ATP-Competitive Inhibitor of mTOR, ATM, and ATR. Cancer Research 73: 2574-2586.
31. Liu Q S, Chang J W, Wang J H, Kang S A, Thoreen C C, et al. (2010) Discovery of 1-(4-(4-Propionylpiperazin-1-yl)-3-(trifluoromethyl)phenyl)-9-(quinolin-3-yl)benzo[h][1,6]naphthyridin-2(1H)-one as a Highly Potent, Selective Mammalian Target of Rapamycin (mTOR) Inhibitor for the Treatment of Cancer. Journal of Medicinal Chemistry 53: 7146-7155.
32. Bharatham N, Chang M W, Yoon H S (2011) Targeting FK506 Binding Proteins to Fight Malarial and Bacterial Infections: Current Advances and Future Perspectives. Current Medicinal Chemistry 18: 1874-1889.
33. Zhang Q, Major M B, Takanashi S, Camp N D, Nishiya N, et al. (2007) Small-molecule synergist of the Wnt/beta-catenin signaling pathway. Proceedings of the National Academy of Sciences of the United States of America 104: 7444-7448.
34. Lomenick B, Hao R, Jonai N, Chin R M, Aghajan M, et al. (2009) Target identification using drug affinity responsive target stability (DARTS). Proceedings of the National Academy of Sciences of the United States of America 106: 21984-21989.
35. Purcell W P (1975) Some Guidelines for Application of Quantitative Structure-Activity-Relationships in Drug Design. European Journal of Medicinal Chemistry 10: 335-339.
36. Dechy-Cabaret O, Benoit-Vical F (2012) Effects of antimalarial molecules on the gametocyte stage of Plasmodium falciparum: the debate. Journal of Medicinal Chemistry 55: 10328-10344.
37. Toye P J, Sinden R E, Canning E U (1977) The action of metabolic inhibitors on microgametogenesis in Plasmodium yoelii nigeriensis. Zeitschrift fur Parasitenkunde 53: 133-141.
38. Yuan J, Johnson R L, Huang R, Wichterman J, Jiang H, et al. (2009) Genetic mapping of targets mediating differential chemical phenotypes in Plasmodium falciparum. Nature Chemical Biology 5: 765-771.
39. Gamo F J, Sanz L M, Vidal J, de Cozar C, Alvarez E, et al. (2010) Thousands of chemical starting points for antimalarial lead identification. Nature 465: 305-U356.
40. Plouffe D, Brinker A, McNamara C, Henson K, Kato N, et al. (2008) In silico activity profiling reveals the mechanism of action of antimalarials discovered in a high-throughput screen. Proceedings of the National Academy of Sciences of the United States of America 105: 9059-9064.
41. Derbyshire E R, Prudencio M, Mota M M, Clardy J (2012) Liver-stage malaria parasites vulnerable to diverse chemical scaffolds. Proceedings of the National Academy of Sciences of the United States of America 109: 8511-8516.
42. Hersey P, Bastholt L, Chiarion-Sileni V, Cinat G, Dummer R, et al. (2009) Small molecules and targeted therapies in distant metastatic disease. Annals of Oncology 20: 35-40.
43. Eccles S A, Massey A, Raynaud F I, Sharp S Y, Box G, et al. (2008) NVP-AUY922: A novel heat shock protein 90 inhibitor active against xenograft tumor growth, angiogenesis, and metastasis. Cancer Research 68: 2850-2860.
44. Garon E B, Moran T, Barlesi F, Gandhi L, Sequist L V, et al. (2012) Phase II study of the HSP90 inhibitor AUY922 in patients with previously treated, advanced non-small cell lung cancer (NSCLC). Journal of Clinical Oncology 30.
45. Pacey S, Wilson R H, Walton M, Eatock M M, Hardcastle A, et al. (2011) A phase I study of the heat shock protein 90 inhibitor alvespimycin (17-DMAG) given intravenously to patients with advanced solid tumors. Clinical cancer research: an official journal of the American Association for Cancer Research 17: 1561-1570.
46. Banumathy G, Singh V, Pavithra S R, Tatu U (2003) Heat shock protein 90 function is essential for Plasmodium falciparum growth in human erythrocytes. The Journal of biological chemistry 278: 18336-18345.
47. Kumar R, Musiyenko A, Barik S (2003) The heat shock protein 90 of Plasmodium falciparum and antimalarial activity of its inhibitor, geldanamycin. Malaria Journal 2: 30.
48. Pesce E R, Cockburn I L, Goble J L, Stephens L L, Blatch G L (2010) Malaria heat shock proteins: drug targets that chaperone other drug targets. Infectious disorders drug targets 10: 147-157.
49. Singh C, Atri N (2013) Chemo-informatic design of antibiotic geldenamycin analogs to target stress proteins HSP90 of pathogenic protozoan parasites. Bioinformation 9: 329-333.
50. Hanson K K, Ressurreicao A S, Buchholz K, Prudencio M, Herman-Ornelas J D, et al. (2013) Torins are potent antimalarials that block replenishment of Plasmodium liver stage parasitophorous vacuole membrane proteins. Proceedings of the National Academy of Sciences of the United States of America.
51. Reyes P, Rathod P K, Sanchez D J, Mrema J E, Rieckmann K H, et al. (1982) Enzymes of purine and pyrimidine metabolism from the human malaria parasite, Plasmodium falciparum. Molecular and biochemical parasitology 5: 275-290.
52. Varvassori S, Wang K, Schweizer L M, Schweizer M (2005) Ramifications of impaired PRPP synthesis in Saccharomyces cerevisiae. Biochemical Society Transactions 33: 1418-1420.
53. Hovejensen B (1989) Phosphoribosylpyrophosphate (Prpp)-Less Mutants of Escherichia-Coli. Molecular microbiology 3: 1487-1492.
54. Tampitag S, O'Sullivan W J (1986) Enzymes of pyrimidine biosynthesis in Crithidia luciliae. Molecular and biochemical parasitology 19: 125-134.
55. Trager W, Jensen J B (2005) Human malaria parasites in continuous culture. 1976. The Journal of parasitology 91: 484-486.
56. Guiguemde W A, Shelat A A, Bouck D, Duffy S, Crowther G J, et al. (2010) Chemical genetics of Plasmodium falciparum. Nature 465: 311-315.
57. Arastu-Kapur S, Ponder E L, Fonovic U P, Yeoh S, Yuan F, et al. (2008) Identification of proteases that regulate erythrocyte rupture by the malaria parasite Plasmodium falciparum. Nature Chemical Biology 4: 203-213.
58. Blagborough A M, Churcher T S, Upton L M, Ghani A C, Gething P W, et al. (2013) Transmission-blocking interventions eliminate malaria from laboratory populations. Nature communications 4: 1812.
59. Wang Y, Jadhav A, Southal N, Huang R, Nguyen D T (2010) A grid algorithm for high throughput fitting of dose-response curve data. Current chemical genomics 4: 57-66.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-47. (canceled)

48. A compound of formula (I): 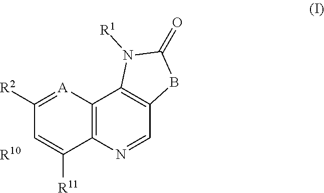
wherein A is CR¹²,
B is CR³═CR⁴,
R¹ is a group selected from the group consisting of C₆₋₁₀ aryl; C₁₋₁₂ alkyl wherein alkyl contains one or more oxygen, sulfur, nitrogen, phosphorus, or silicon atoms in place of carbon atoms; 5-10-membered heteroaryl having 1-4 heteroatoms independently selected from the group consisting of nitrogen, oxygen, and sulfur; and 4-7-membered heterocyclic having 1-2 heteroatoms independently selected from the group consisting of nitrogen, oxygen, and sulfur; wherein the alkyl containing one or more oxygen, sulfur, nitrogen, phosphorus, or silicon atoms, aryl, or heteroaryl is optionally substituted with one substituent selected from trifluoromethyl, C₁-C₆ alkyl, halo, CN, C₁-C₆ alkoxy, SO₂NH₂, piperazinyl, and 4-alkylcarbonylpiperazinyl,
R² is selected from 2-amino-5-pyridinyl, 4-pyridinyl, 2-amino-5-pyrimidinyl, 3-pyridyl, quinolin-3-yl, 5-pyrimidinyl, 2-amino-5-trifluoromethylpyrimidin-5-yl, 2-acetylamino-5-pyridyl, 2-amino-4-methylpyrimidin-5-yl, 1-piperazinyl, indol-5-yl, 1H-indazol-5-yl, 4-aminophenyl, 1,2,3,6-tetrahydropyridin-4-yl, 1H-pyrazol-4-yl, 1H-benzo[d]imidazol-5-yl, 4-sulfonylaminophenyl, 2-dimethylaminopyrimidin-5-yl, 3-trifluoromethylphenyl, bromo, 3-aminophenyl, vinyl, 4-aminocarbonylphenyl, 3-cyanophenyl, 3-trifluoromethyl-5-pyridyl, tetrazolyl, 4-chlorophenyl, 4-methoxyphenyl, 3-aminocarbonylphenyl, 3-acetylphenyl, 2,3-dihydrobenzofuran-6-yl, 1-methyl-1H-indol-5-yl, benzo[d][1,3]dioxo-5-yl, 4-fluorophenyl, 4-hydroxyphenyl, porpholin-1-yl, benzo[b]thiophen-1-yl, 4-methylsulfonylphenyl, benzo[c][1,2,5]oxadiazol-5-yl, 2-(piperidin-1-yl)-3-pyridinyl, 4-carboxyphenyl, 2-methyl-5-pyridyl, 4-dimethylaminocarbonylphenyl, 4-phenylphenyl, 4-methylpenyl, 3-chloro-5-pyridyl, (3-pyrrolidin-1-yl)phenyl, 4-([piperizin-1-yl]carbonyl)phenyl, 4-([morpholin-1-yl]carbonyl)phenyl, 2-hydroxypyrimidin-5-yl, 3-aminosulfonylphenyl, 2-oxo-1,2,3,4,tetrahydroisoquinolin-6-yl, 2-oxo-1,2,3,4,-tetrahydroquinolin-6-yl, 4-(aminomethyl)phenyl, 4-(dimethylaminomethyl)phenyl, 4-(methylaminocarbonyl)phenyl, 1-oxoindolin-5-yl, and 1-oxoisoindolin-5-yl,
R¹¹ and R¹² are hydrogen,
R³ and R⁴ are independently selected from hydrogen, hydroxyl, OR⁵, halogen, optionally substituted C₆₋₁₀ aryl, and optionally substituted C₁₋₆ alkyl,
R⁵ is C₁₋₁₂ alkyl, and
R¹² is hydrogen, C₁₋₁₂ alkyl, C₆₋₁₀ aryl, halogen, hydroxyl, or OR⁵,
or a pharmaceutically acceptable salt thereof,
with the proviso that when R³, R⁴, R¹⁰, and R¹¹ are each hydrogen, and R¹ is 3-trifluoromethylphenyl, R² is not 2-amino-5-pyridyl or 3-quinolinyl.

49. The compound of claim 48, wherein
R¹ is C₆-C₁₀ aryl or heteroaryl, wherein the alkyl, aryl, or heteroaryl is optionally substituted with one or more substituents selected from trifluoromethyl, C₁-C₆ alkyl, halo, CN, C₁-C₆ alkoxy, SO₂NH₂, piperazinyl, and 4-alkylcarbonylpiperazinyl,
R¹⁰ and R¹¹ are both hydrogen, and
R³ and R⁴ are individually selected from hydrogen, halo, optionally substituted C₁-C₆ alkyl, and OR⁵.

50. The compound of claim 48, wherein R³ and R⁴ are both hydrogen.

51. The compound of claim 48, wherein R¹ is selected from 3-trifluoromethylphenyl, 3-ethylphenyl, 4-piperazinyl, 1-acetylpiperidin-4-yl, and 4-tetrahydropyranyl.

52. The compound of claim 51, wherein R² is selected from 2-amino-pyridinyl, 4-pyridinyl, 2-amino-5-pyrimidinyl, 3-pyridyl, quinolin-3-yl, 5-pyrimidinyl, 2-acetylamino-5-pyridyl, 2-amino-4-methylpyrimidin-5-yl, indol-5-yl, 1H-indazol-5-yl, 4-aminophenyl, 1,2,3,6-tetrahydropyridin-4-yl, 1H-pyrazol-4-yl, 1H-benzo[d]imidazol-5-yl, 4-sulfonylaminophenyl, 2-dimethylamninopyrimidin-5-yl, 3-trifluoromethylphenyl, bromo, 3-aminophenyl, vinyl, 4-aminocarbonylphenyl, 3-cyanophenyl, tetrazolyl, 4-chlorophenyl, 4-methoxyphenyl, 3-acetylphenyl, 2,3-dihydrobenzofuran-6-yl, 1-methyl-1H-indol-5-yl, benzo[d][1,3]dioxo-5-yl, 4-fluorophenyl, 4-hydroxyphenyl, benzo[b]thiophen-1-yl, 4-methylsulfonylphenyl, benzo[c][1,2,5]oxadiazol-5-yl, 2-(piperidin-1-yl)-3-pyridinyl, 4-carboxyphenyl, 2-methyl-5-pyridyl, 4-methylsulfonylphenyl, 4-dimethylaminocarbonylphenyl, 4-phenylphenyl, 4-methylpenyl, 3-chloro-5-pyridyl, (3-pyrrolidin-1-yl)phenyl, 4-([piperizin-1-yl]carbonyl)phenyl, 4-([morpholin-1-yl]carbonyl)phenyl, 3-aminosulfonylphenyl, 2-oxo-1,2,3,4,tetrahydroisoquinolin-6-yl, 2-oxo-1,2,3,4,-tetrahydroquinolin-6-yl, 4-(methylaminocarbonyl)phenyl, 1-oxoindolin-5-yl, and 1-oxoisoindolin-5-yl.

53. A compound of formula (II): 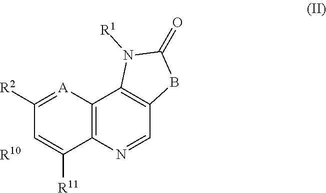
wherein A is CR¹²,
B is NR¹³,
R¹ is an optionally substituted group selected from the group consisting of C₆₋₁₀ aryl; C₁₋₁₂ alkyl; C₁₋₁₂ alkyl wherein alkyl contains one or more oxygen, sulfur, nitrogen, phosphorus, or silicon atoms in place of carbon atoms; wherein the C₁₋₁₂ alkyl, C₁₋₁₂ alkyl wherein alkyl containing one or more oxygen, sulfur, nitrogen, phosphorus, or silicon atoms, or aryl is optionally substituted with one or more substituents selected from trifluoromethyl, C₁-C₆ alkyl, halo, CN, C₁-C₆ alkoxy, SO₂NH₂, piperazinyl, and 4-alkylcarbonylpiperazinyl,
R² is selected from 2-amino-5-pyridinyl, 4-pyridinyl, 2-amino-5-pyrimidinyl, 3-pyridyl, quinolin-3-yl, 5-pyrimidinyl, 2-amino-5-trifluoromethylpyrimidin-5-yl, 2-acetylamino-5-pyridyl, 2-amino-4-methylpyrimidin-5-yl, 1-piperazinyl, indol-5-yl, 1H-indazol-5-yl, 4-aminophenyl, 1,2,3,6-tetrahydropyridin-4-yl, 1H-pyrazol-4-yl, 1H-benzo[d]imidazol-5-yl, 4-sulfonylaminophenyl, 2-dimethylaminopyrimidin-5-yl, 3-trifluoromethylphenyl, bromo, 3-aminophenyl, vinyl, 4-aminocarbonylphenyl, 3-cyanophenyl, 3-trifluoromethyl-5-pyridyl, tetrazolyl, 4-chlorophenyl, 4-methoxyphenyl, 3-aminocarbonylphenyl, 3-acetylphenyl, 2,3-dihydrobenzofuran-6-yl, 1-methyl-1H-indol-5-yl, benzo[d][1,3]dioxo-5-yl, 4-fluorophenyl, 4-hydroxyphenyl, porpholin-1-yl, benzo[b]thiophen-1-yl, 4-methylsulfonylphenyl, benzo[c][1,2,5]oxadiazol-5-yl, 2-(piperidin-1-yl)-3-pyridinyl, 4-carboxyphenyl, 2-methyl-5-pyridyl, 4-dimethylaminocarbonylphenyl, 4-phenylphenyl, 4-methylphenyl, 3-chloro-5-pyridyl, (3-pyrrolidin-1-yl)phenyl, 4-([piperizin-1-yl]carbonyl)phenyl, 4-([morpholin-1-yl]carbonyl)phenyl, 2-hydroxypyrimidin-5-yl, 3-aminosulfonylphenyl, 2-oxo-1,2,3,4,tetrahydroisoquinolin-6-yl, 2-oxo-1,2,3,4,-tetrahydroquinolin-6-yl, 4-(aminomethyl)phenyl, 4-(dimethylaminomethyl)phenyl, 4-(methylaminocarbonyl)phenyl, 1-oxoindolin-5-yl, and 1-oxoisoindolin-5-yl,
R¹⁰ and R¹¹ are hydrogen,
R⁵ is C₁₋₁₂ alkyl, and
R¹² is hydrogen, C₁₋₁₂ alkyl, C₆₋₁₀ aryl, halogen, hydroxyl, or OR⁵, and
R¹³ is hydrogen, C₁₋₁₂ alkyl or C₆₋₁₀ aryl,
or a pharmaceutically acceptable salt thereof.

54. The compound of claim 53, wherein R¹⁰ and R¹¹ are both hydrogen.

55. The compound of claim 53, wherein R¹³ is hydrogen or C₁₋₁₂ alkyl.

56. The compound of claim 55, wherein R¹ is 3-trifluoromethylphenyl.

57. The compound of claim 55, wherein R² is selected from the group consisting of 2-methyl-5-pyridyl, 4-aminophenyl, 2-acetylamino-5-pyridyl, 4-hydroxyphenyl, 3-aminophenyl, 4-pyridyl, 1H-benzo[d]imidazol-5-yl, 4-methylsulfonylphenyl, quinolin-3-yl, 2-aminopyrimidin-5-yl, 3-cyanophenyl, 3-pyridyl, and 4-aminocarbonylphenyl.

58. A pharmaceutical composition comprising the compound or salt of claim 48 and a pharmaceutically acceptable carrier.

59. A pharmaceutical composition comprising the compound or salt of claim 53 and a pharmaceutically acceptable carrier.

60. A method of blocking transmission of a Plasmodium parasite comprising administering to a mammal in need of such treatment, a therapeutically effective amount of a first compound of claim 48 or a pharmaceutically acceptable salt thereof, optionally in combination with a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat.

61. The method of claim 60, wherein the second compound is elesclomol, NSC174938, NVP-AUY922, maduramicin, and narasin.

62. The method of claim 60, wherein the Plasmodium parasite is in a gametocyte stage.

63. A method of treating malaria by killing or arresting the growth of Plasmodium organisms in a mammal, wherein the Plasmodium organisms are in a gametocyte stage, the method comprising administering to a mammal a therapeutically effective amount of a compound of claim 53 or a pharmaceutically acceptable salt thereof, optionally in combination with a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat.

64. The method of claim 63, wherein the second compound is elesclomol, NSC174938, NVP-AUY922, maduramicin, and narasin.

65. The method of claim 63, wherein the Plasmodium gametocyte is a stage III-V gametocyte.

66. A method of providing prophylaxis to a mammal in need thereof against malaria, comprising administering to the mammal a therapeutically effective amount of a first compound of claim 48 or a pharmaceutically acceptable salt thereof, optionally in combination with a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat.

67. A method of providing prophylaxis to a mammal in need thereof against malaria, comprising administering to the mammal a therapeutically effective amount of a first compound of claim 53 or a pharmaceutically acceptable salt thereof, optionally in combination with a second compound selected from elesclomol, NSC174938, NVP-AUY922, maduramicin, narasin, alvespimycin, omacetaxine, thiram, zinc pyrithione, phanquinone, bortezomib, salinomycin sodium, monensin sodium, dipyrithione, dicyclopentamethylene-thiuram disulfide, YM155, withaferin a, adriamycin, romidepsin, AZD-1152-HQPA, CAY10581, plicamycin, CUDC-101, auranofin, trametinib, GSK-458, afatinib, and panobinostat.
